(19) United States
(12) Patent Application Publication
(10) Pub. No.: US 2026/0137949 A1
(43) Pub. Date: May 21, 2026

(54) Pulsed Low Frequency Magnetic Fields And Tumor Membrane Disruption

(71) Applicant: The United States Government As Represented By The Department of Veterans Affairs, Washington, DC (US)

(72) Inventors: Mark M. Fuster, San Diego, CA (US); James Friend, San Diego, CA (US)

(21) Appl. No.: 19/396,887

(22) Filed: Nov. 21, 2025

Related U.S. Application Data
(60) Provisional application No. 63/723,345, filed on Nov. 21, 2024.

(51) Int. Cl. A61N 2/00; A61N 2/02; A61N 5/00; G01N 33/50

(52) U.S. Cl. CPC A61N 2/002; A61N 5/00; G01N 33/5011; G01N 33/5091; A61N 2/02

(57) ABSTRACT

Tumor glycocalyx may be regarded as a glycan “canopy” above the tumor plasma membrane. Disclosed are methods of treating a neoplastic malignant cell cancer wherein the neoplastic malignant cancer cell has a malignant glycocalyx B. Also disclosed are methods of inducing apoptosis of one or more neoplastic malignant cells and methods of increasing a subject's responsiveness to an immunotherapy wherein the neoplastic malignant cancer cell has a malignant glycocalyx B.

### CROSS-REFERENCE TO RELATED APPLICATIONS

This application claims benefit of U.S. Provisional Application No. 63/723,345 filed Nov. 21, 2024 and is hereby incorporated herein by reference in its entirety.

### BACKGROUND

A growing literature has demonstrated unique and sometimes striking effects of electromagnetic (EM) fields on tumor cells and animal experimental tumors (Huang et al 2023; Kielbik et al 2021; Tatarov et al 2011; Tofani et al 2001; Nie et al 2013; Koh et al 2008; Zhang et al 2002; Hambarde et al 2023). This includes exposures to some human and large-animal tumors under compassionate therapy platforms with encouraging responses (Sharma et al 2019; Barbault et al 2009; Vasishta et al 2010; Garcia et al 2017) while signaling a need to examine these phenomena in rational studies guided by an improved understanding of mechanisms.

What is needed are methods of treating neoplastic malignant cell cancers with pulsed low frequency magnetic fields in neoplastic malignant cell cancer.

### BRIEF SUMMARY

Disclosed are methods of treating a neoplastic malignant cell cancer in a subject in need thereof comprising: a) obtaining or having obtained a sample from the subject, wherein the sample comprises at least one neoplastic malignant cancer cell; b) determining that the isolated neoplastic malignant cancer cell comprises a malignant glycocalyx B; and c) applying a pulsed magnetic field (PMF) to the subject, thereby treating the neoplastic malignant cell cancer in the subject.

Disclosed are methods of treating a malignant neoplasm in a subject in need thereof comprising: a) obtaining or having obtained a sample from the subject, wherein the sample comprises at least one neoplastic malignant cancer cell; b) determining that the isolated neoplastic malignant cancer cell comprises a malignant glycocalyx B; and c) applying a pulsed magnetic field (PMF) to the subject, thereby treating the neoplastic malignant cell cancer in the subject.

Disclosed are methods of inducing apoptosis of one or more neoplastic malignant cells comprising: a) determining that the one or more neoplastic malignant cells comprises a malignant glycocalyx B; and b) applying a pulsed magnetic field (PMF) to the one or more neoplastic malignant cells, thereby inducing apoptosis of one or more of the neoplastic malignant cells.

Disclosed are methods of increasing a subject's responsiveness to an immunotherapy, comprising: a) obtaining or having obtained a sample from the subject, wherein the sample comprises at least one neoplastic malignant cancer cell; b) determining that the isolated neoplastic malignant cancer cell comprises a malignant glycocalyx B; and c) applying a pulsed magnetic field (PMF) to the subject, thereby increasing a subject's responsiveness to an immunotherapy.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A-1D show the effects of unique Lf-PMF exposures on membrane leak of brain tumor cells: (FIG. 1A) Monolayer plated cells of the Sia over-expressing cell lines T98G (glioblastoma) and SHSY (neuroblastoma) plated at near-confluence were exposed to a very low-intensity Lf-PMF solenoid system, delivering a 20 mT maximum field (dB/dt˜2 T/sec) over a 10 msec duty cycle at 50 Hz. After exposure, cells were immediately assayed for protease leak using a commercial luciferase-based assay that detects free proteases released into the medium. There was modest but significant leak detected after a 5-min exposure to the pulse train; *P<0.05 (T98G cells) and **P<0.01 (SHSY cells) for difference in means compared to control (normalized data from n=3 separate experiments for each cell line). (FIG. 1B) Data from representative single-plate experiments for the same cell lines exposed to 5 min of a high dB/dt MagVenture system (driving Lf-PMF pulse trains with 80 T/sec pulses over a 70 sec duty cycle at 15 Hz) is shown: Normalized differences in mean responses relative to control are shown for T98G cells (left graph; for n=5 control wells and n=9 Lf-PMF exposed wells, ***P<0.001 for difference) and for SHSY cells (right graph; n=6 control wells and n=9 Lf-PMF exposed wells, **P<0.01 for difference). (FIG. 1C) Illustrations of MagVenture coil system, used for cells as in (FIG. 1), with concept-cartoon demonstrating a side view of plated cells within a pulsing magnetic field running perpendicular through cell layers on 96-W plate. The plate is placed at the edge of the trough-shaped coil on its side (“C” shape side-view to left; field strength shown as “B,” and generated EMFs induced by dB/dt; and with intensity falling off to the right with distance). For separate experiments of plated cells over a low strength solenoid system, used for cells as in (FIG. 1A), instead of a high dB/dt MagVenture coil, the cylindrical solenoid underneath the plated cells generates low dB/dt EMFs wherein low-strength/longer duty-cycle B-field lines (vertical arrows) pulse from the solenoid core-platform perpendicularly through plated cells. (FIG. 1D) Mean data from multiple experiments using the high dB/dt MagVenture system: Differences in mean responses normalized to control are shown for T98G cells (left bar; for n=6 independent experiments; *P<0.01 for difference versus control) and SHSY cells (right bar; for n=5 independent experiments; *P=0.055 for difference).

FIG. 2 shows the effect of a high performance Lf-PMF field on membrane leak in malignant cells and unique primary cells. Cells in monolayer 96-well dishes at ˜80% confluence were treated for 5 min at room temp to Lf-PMF exposures pulsed at 15 Hz using a MagVenture coil placed in lateral orientation allowed for exposure of cells to two magnetic field flux strengths by examining 9-well clusters in domains that are close (within 4 cm) or far (within 10 cm) from the edge of the pulsing source (i.e., trough-coil oriented on its side). This allowed the Lf-PMF pulses with field-lines running perpendicular to plated monolayers to expose near- and far-cells at two flux strengths: (i) 80 T/sec flux strength or (ii) flux strength of 1.8 T/sec (both using 15 Hz pulses delivered at duty cycle of 70 sec/pulse). Control cells were held at room temp without Lf-PMF exposure for the same period. After the 5 min exposure period, cells were immediately assayed for protease leak using a commercial (Promega) luciferase-based assay that detects free proteases released into the culture medium. Cell leak was quantified normalized to that of control cells. Data represents mean of 9 wells for each magnetic flux-strength per cell line, while respective controls were quantified from mean of 6 wells for most lines (three wells for A549 cells). Far right: dendritic immune cells, as a primary cell line differentiated from bone marrow, were tested as a non-tumor primary cell line. Significance for the differences in means between groups in graphs for tumor cells are indicated on the graph as *** P<0.001, ** P<0.001, *P<0.05 for the indicated differences.

FIGS. 3A-3D show the impact of Sia in the glycocalyx on Lf-PMF leak: Effect of AUS sialidase Sia elimination. (FIG. 3A) Lf-PMF exposure (5 min MagVenture pulses at 80 T/sec dB/dt using 15 Hz train) results in protease leak (middle bar) normalized to baseline, while protease leak following treatment of SHSY cells with AUS sialidase (which removes cell-surface Sia) was sufficient to abrogate the Lf-PMF mediated leak (right bar); **P<0.01, ***P<0.001 for difference in means, n=6 wells for control and n=9 wells for Lf-PMF exposure in each experiment (data representative of 4 independent experiments). (FIG. 3B) Mean reduction in Lf-PMF induced membrane leak achieved by AUS sialidase pre-treatment in T98G cells and SHSY tumor cells was assessed in multiple trials: graph shows mean inhibition in Lf-PMF leak as a result of AUS pre-treatment; **P<0.001 for mean inhibition relative to non-AUS Lf-PMF exposed cells; mean of n=3 experiments for T98G cell line and n=4 experiments for SHSY cell line. (FIG. 3C) Illustration of AUS sialidase elimination of anionic tumor-surface Sia (noted as “SA” in figure text) on complex branched glycans decorating tumor glycoproteins. AUS digests α2,3 or α2,6 glycosidic linkages that link terminal Sia (diamond) to galactose residues (circle) on heavily branched terminal glycans on the cancer-cell surface (zoom inset), over-expressed on tumor glycoproteins (including mucins). (FIG. 3D) To assess the presence and reduction in surface Sia in AUS-treated SHSY cells, flow cytometry was carried out using fluorescent lectins (MAL-II and SNL) to bind with terminal Sia expressing glycans on the surface of SHSY cells. Right graph shows marked SNL binding (depends on cell-surface α2,6 linked Sia), comparing far-right SNL (+) control to neg control at left. AUS reduced SNL binding by˜70% (leftward shift in “AUS” curve on log scale). MAL-II binding (depends on α2,3 linked Sia) was >60% reduced (left graph; noting leftward shift in “AUS” curve relative to (+) control.

FIGS. 4A-4B shows the concept and biophysical basis for induction forces on the cancer glycocalyx during magnetic field pulsing. (FIG. 4A) Target molecules for magnet-induced EMF motion and resultant tumor cell membrane disruption are charged glycans over-expressed in a glycocalyx “canopy” above the plasma membrane of carcinoma cells. These include: (i) proteoglycans (left), composed of anionic sulfated glycosaminoglycans such as heparan sulfate (HS) and chondroitin sulfate (CS) attached to core proteins: HS and CS chains are shown O-linked via xylose to the core protein, modified by anionic N-sulfation and 2S, 6S, and/or 3S sulfation (on HS) or 4S sulfation (on CS). Anionic charge (red dashes) on heavily sulfated glycan domains over-expressed on the plasma membrane of several cancer subtypes mediate HS binding to basic amino acids on chemokines, tumor growth factors, and receptors; (ii) cell-surface sialic acid residues “cap” branched terminal glycans on carcinoma membrane proteins (right), typically expressed of O-linked mucin glycoproteins. They are also highly expressed on malignant membrane polysialic acid (e.g., on small cell lung cancer and brain tumor cells). Application of external oscillating magnetic fields (vertical field-lines; magnitude B), and induced EMFs proportional to dB/dt, where dt is the pulse interval: Induced EMF/voltage in diagram is shown as potential conduction paths (blue) orthogonal to pulsed magnetic field lines, engaging with charged/anionic tumor glycans in the glycocalyx, imparting force F (red arrows) on core proteins, and creating torque (Tau symbol) about points anchoring core proteins to the membrane (red curved arrows). This may induce leak (wavy grey lines) of small molecules such as proteases and tumor cell stress. Glycan legend (Xyl: Xylose, Gal: Galactose, GlcNAc: N-acetylglucosamine, IdoA: iduronic acid, GlcA: glucuronic acid, GalNAc: N-acetylgalactosamine, Sia: Sialic acid, Fuc: Fucose, Man: Mannose). (FIG. 4B) To measure membrane leak using an alternate method, examining inward leak of an exogenous standard indicator (propidium iodide; PI), standard Lf-PMF exposure of model A549 tumor cells to the solenoid field platform in the presence of exogenous PI indicated significant inward leak (right bar, normalized to control cells; n=6 trials, *P=0.05 for difference versus control). This was comparable to the magnitude noted for outward leak of protease (using CytoTox assay system) in the same cells under the same exposure conditions (middle bar, normalized to control; n=6 trials, **P<0.01 for difference versus control).

FIGS. 5A-5C show lf-PPMF mediated leak and roles for major EMF transducing anionic glycocalyx component glycans. (FIG. 5A) Lf-PMF exposures (5 min MagVenture pulses at 80 T/sec dB/dt using 15 Hz train) were carried out in 96W plates (as in FIG. 1B) using SHSY neuroblastoma cells, resulting in significant protease leak over that of control cells (graph, middle bar; mean of n=4 experiments; normalized to non-exposed control cells; *P<0.056 for mean relative to control), while Lf-PMF exposure following treatment of SHSY cells with AUS sialidase was sufficient to nearly abrogate the Lf-PMF induced leak (right bar; **P<0.01 for difference between mean and that of Lf-PMF exposure). To right, cartoon shows membrane proteins projecting charged glycans (anionic charge depicted by circled “-” signs) overexpressed by tumor cells into the glycocalyx, depicted as a rectangular “slab” above tumor membrane, shown as a long floating box. The conceptual example depicts a predominance of glycoproteins with anionic Sia (diamond) terminal monosaccharide modifications, or polymeric Sia (polysialic acid) that modify glycoproteins surrounding a representative proteoglycan shown to right of center with anionic sulfated glycosaminoglycan chains. This “Sia-dominant” model may represent the typical surface of a brain tumor cell. A hypothetical pulse-induced EMF (top grey arrow) is shown engaging with the anion-rich glycocalyx to impart mechanical rightward force on the slab, creating membrane shear stress (via torque on transmembrane attachments), solute leak at the base (black arrows), and downstream cell stress. (FIG. 5B) Lf-PMF exposures were carried out on A549 lung adenocarcinoma monolayers. A significant increase in Lf-PMF induced leak over that of non-exposed control cells was noted, shown in graph (middle bar; mean of n=3 experiments; normalized to baseline; *P=0.013 for mean relative to control). Treatment of A549 cells with heparin lyase (Hep'ase) was sufficient to completely abrogate the Lf-PMF mediated leak (right bar; **P<0.01 for difference between mean and that of Lf-PMF exposure response). To right, hypothetical scenario for A549 glycocalyx exposed to Lf-PMF, where proteoglycan membrane proteins project a dominance of anionic sulfated HS glycosaminoglycan polymers into the rectangular glycocalyx “slab.” Shown is pulse-induced EMF (arrow) now engaging with glycosaminoglycan rich glycocalyx to impart rightward force on slab. (FIG. 5C) Flow cytometry analysis of fluorescent FGF-2 binding to A549 and SHSY tumor cells as a probe for cell-surface HS glycosaminoglycans, and the effect of heparin lyase (Hep'ase). For A549 cells (left graph), marked shift in FGF-2 binding is shown (right-most histogram) relative to negative control (far left). Treatment of cells with Hep'ase shows reduction in FGF-2 binding (middle histogram, “+FGF2 Post Hep'ase”). For SHSY cells (right graph), a markedly lesser shift in FGF-2 binding is shown, with leftward shift in post-Hep'ase.

FIG. 6 shows the biophysical basis for induction forces on the cancer glycocalyx during magnetic field pulsing: Target molecules for magnet-induced EMF motion and resultant tumor cell membrane disruption are charged glycans of a unique glycocalyx expressed on carcinoma cells. These include: (i) proteoglycans (left), composed of anionic sulfated glycosaminoglycans such as heparan sulfate (HS) and chondroitin sulfate (CS) attached to core proteins (e.g., syndecans and glypicans or CSPG4 as carcinoma HS or CS proteoglycans): HS and CS chains are shown O-linked via xylose to the core protein, modified by anionic N-sulfation and 2S, 6S, and/or 3S sulfation (on HS) or 4S sulfation (on CS). Anionic charge (dashes) on heavily sulfated glycan domains over-expressed on the plasma membrane of several cancer subtypes (including lung cancer) mediate HS binding to basic amino acids on chemokines, tumor growth factors, and receptors; (ii) cell-surface sialic acid residues “cap” branched terminal glycans on carcinoma membrane glycoproteins (right), typically expressed of O-linked mucin glycoproteins. They are also highly expressed on malignant membrane polysialic acid (e.g., on small cell lung cancer and brain tumor cells), and drive invasion via cell-cell anionic repulsion. Glycan legend (Xyl: Xylose, Gal: Galactose, GlcNAc: N-acetylglucosamine, IdoA: iduronic acid, GlcA: glucuronic acid, GalNAc: N-acetylgalactosamine, Sia: Sialic acid, Fuc: Fucose, Man: Mannose).

FIGS. 7A-7D show sulfated glycans on A549 lung cancer cell glycocalyx mediate Lf-PMF induced leak and cell stress: A549 lung tumor cells grown in 96W plates to 80% confluence were exposed to a pulsed solenoid platform at room temp with a Lf-PMF field of magnitude 20 mT, and pulsed at 50 Hz for 5 min (“Pulsed Solenoid” condition), while control non-exposed cells in identical monolayer conditions were left at room temp over the same period. (FIG. 7A) Protease release using a commercial cell-membrane injury assay (Promega cytotoxicity assay) was measured immediately after exposure, with graph showing a boost in protease release in Lf-PMF exposed cells, and heparanase pre-treatment of Lf-PMF exposed cells resulting in a loss of leak effect (right bar); *P<0.05 for difference in means relative to control. (There was no significant effect of heparanase treatment by itself on protease release by control, non-magnet exposed cells; not shown.) (FIG. 7B) Viability of control versus 5-min Lf-PMF train exposed cells was examined at 4 hr post-exposure after cells were harvested from standard culture conditions using a commercial (Promega) metabolic activity based assay, with graph showing mean relative viability signal post-Lf-PMF treatment compared to controls (mean of 4 experiments; #P=0.06 for difference). (FIG. 7C) Cell growth in culture at 3 days following the 5-min Lf-PMF exposure was assessed, with comparison to control cells. Graph shows cell counts at day 3 following Lf-PMF versus control exposure of cells (seeded originally at 25,000/well in 12-well plates; *P<0.05 for difference in means). (FIG. 7D) Schematic of tumor over-expression of anionic, sulfated glycosaminoglycan chains tethered to proteoglycans, projecting into the lung cancer cell glycocalyx: HS lyase (Heparinase III) digestion renders cells insensitive to the Lf-PMF effect, providing mechanistic insight.

FIGS. 8A-8B show proof of concept on effects of Lf-PMF exposure on membrane leak and late viability of high-SA tumor cells: (FIG. 8A) Leak normalized to control was examined in SA over-expressing tumor cell lines SHSY (neuroblastoma) and T98G (glioblastoma) exposed to 5 min of a MagVenture driven (8.0 kT/sec 15 Hz) Lf-PMF pulse train versus control/non-magnet exposure period at room temp; **P<0.01, ***P<0.001 for difference in means among n=5 wells for control and n=9 wells for Lf-PMF exposure in each experiment (representative of 3 independent experiments for each line). (FIG. 8B) Mean cell growth inhibition in culture at 3 days following the short 5-min Lf-PMF exposure remains significant (% relative to control) for each cell line, with findings carried out in a mean of n=4 trials for SHSY cells (*P<0.05 for difference relative to control mean, one-sided 2-tailed T-test) and n=3 trials for T98G cells (**P<0.01 for difference versus control).

FIG. 9 shows a schematic and biophysics of advanced Lf-PMF magnetic field delivery system for planned pilot exposures: A tumor cell monolayer shown above a magnetic platform is exposed to repeated pulsed circuit driven magnetic field lines (pulsed through the cell layer), inducing EMFs that may generate torsional forces on charged tumor-glycocalyx molecule, including anionic proteoglycans (e.g., sulfated GAG, “T” shapes on cell surfaces) or sialic acid (diamond shape) modifications that “cap” cell-surface glycoproteins: Either or both are commonly overexpressed on lung tumor cells. Greater magnitude EMFs are achieved by greater dB/dt or a narrow pulse width (dt). At right: Effects on tumor cells, which may include membrane leak and altered tumor cell viability. Visualizing pulse-driven motion of tumor glycocalyx is one of the Aim-I goals in this proposal.

FIGS. 10A-10C show two major electromagnetic (EM) platform strategies used to affect tumor behavior to date. Mechanisms of tumor cell engagement are only partially understood and discussed in the main text. In one family of EM platforms, electromagnetic waves (FIG. 10A) are delivered as traveling waves in space and characterized by frequency (v), wavelength (λ) (with product c, the speed of light) as well as power density, and direction defined by the Poynting vector (S, proportional to cross-product of E- and B-fields) shown below the illustrated EM wave with orthogonal electric and magnetic vector components, oscillating at frequencies emitted typically by radiofrequency-generating equipment to affect tumor cell biological behavior or even inflammatory tissue remodeling. While attenuated by interaction with tissue, the ability of such radiation to affect deeply seated tumor cells is governed by the dielectric properties of tissue. Frequencies that may affect tumor cell viability in plated tumor-cell studies may extend beyond the radiofrequency range and to the high non-ionizing range (infrared or even vis-light range; bottom). (FIG. 10B) In pulsed delivery systems, pulsed EM wave “packets” may be delivered as pulsed electromagnetic fields (PEMFs); alternatively, a pulsed magnetic field (PMF) may be applied to generate induced electromotive forces (EMFs; voltage fields orthogonal to the pulsing external B-field) deep in tissue through which the time-dependent magnetic field, B(t), penetrates, with graphed pulse-train at left, showing the pulse profile (dB/dt as the magnitude of DB delivered over Dt, the pulse-width; pw), with pulses delivered at arbitrary frequency, f (Hz), often at ultra-low pulse driving frequencies (<300 Hz). Middle illustration shows how the induced EMF, proportional to dB/dt (change in magnetic flux), can drive motion of charge element q with mass m along induced electric field vector E on the surface of a hypothetical tumor cell (e.g., glycocalyx charge motion) or even a “slab” of charge on a spherical cluster of tumor cells. The same pulsed forces on the tumor glycocalyx (to which charge q and mass m are attached) may create shear on the tumor cell surface (right illustration) at molecular attachments to the membrane, creating tumor cell stress and downstream viability effects. (FIG. 10C) Theoretically, a variety of tumor cells (T) with unique cell surface charge (anionic surfaces as illustrated) in a population may be susceptible to pulsed EMFs, distinctly governed by the charged areas over which dB/dt fluxes (designated as hypothetical EMF1, EMF2, etc., in red), wherein differential intra-tumoral effects and stress are possible.

FIG. 11 shows a summary of biophysical barriers to potential augmented anti-tumor immunologic priming through electromagnetic (EM) perturbations of the tumor microenvironment. Each barrier is indicated in the center (left column) along with descriptions of physical perturbations that may be achieved through EM platform exposure (middle column) and potential specific immunologic events (right column) that may be augmented through EM platform exposures that can (directly or indirectly) modify or eliminate barrier condition(s). For pictorial attachments corresponding to each barrier, the upper left side of each illustration states the barrier and illustrates the nature of the barrier under tumor-pathologic conditions, while lower right (indicated by a pulsed-field symbol) illustrates modified conditions that correspond to post-EM exposure effects on the respective barrier. This illustrates how immunologic priming or “access” of immune cells (whether naïve or sensitized) may be augmented by the specific effect(s) of EM exposure on the specific barrier under consideration.

FIG. 12 shows induction of non-thermal and non-ionizing electromagnetic force in the tumor microenvironment: potential consequences of glycocalyx disruption. Tumor cells (top and to left) are illustrated in a growing region on the edge of carcinoma, with figure as a zoomed inset in lower-left cartoon of vascularized tumor mass. Tumor cells are characterized by thick surface glycocalyces, as illustrated by dense regions of wavy lines surrounding the plasma membrane, as in tumor cells at baseline (left side) while surrounded by extracellular matrix (ECM) polymers rich in heparan sulfate glycosaminoglycan (HS-GAG) chains, represented by extracellular wavy lines to right of cell. Also shown nearby is a vascular remodeling region depicting lymphangiogenic responses to tumor vascular growth factors (GFs; such as VEGF family members) as well as integrins (Int), growing into center. Nearby immune cells such as naïve T cells (T) or activated T cells (T*) and antigen-presenting cells (APCs) may be sensitized against tumor antigens (T-Ag) in this tumor microenvironment. As an EM platform is applied in this case as an external pulsed magnetic field depicted by vertical light gray lines across center and right side of figure; B(t), the induced EMF shown in blue, with circular field lines orthogonal to the pulsed magnetic field (and proportional to dB/dt), engages with the charge-rich tumor glycocalyx (tumor cell at top, within field), distorting it and inducing leak on the tumor membrane (gaps in membrane of cell). This may induce tumor cell stress and release of tumor antigens (T-Ag; small gray and black ovals) and promote CD8+ T-cell sensitization and activation by nearby APCs, which may cross-present T-Ag on MHC-I (illustrated by blue/red APC receptor). This illustrates one way by which anti-tumor immunity may be augmented by an EM platform of pulsed magnetic fields applied to the tumor microenvironment.

FIGS. 13A-B shows tumor glycocalyx and temporal dynamics of applying a pulsed magnetic field (PMF) EM platform. (FIG. 13A) Short-term effects of the PMF include possible lateral forces as well as molecular torque-induced stress and membrane leak resulting from the induced EMF that pulses at the driving frequency. Note perforations or “pores” along tumor (upper right scenario) resulting from movement of a dense anionic glycocalyx “slab” that consists of overexpressed hyaluronan (top layer of charged polymers) as well as other heavily expressed glycans such as GAG chains (e.g., heparan sulfate) and complex branched Sia capped glycoproteins and glycolipids/gangliosides in the glycocalyx “canopy” that decorates core proteins and lipids attached at the tumor plasma membrane base. An additional consequence of this short-term membrane-disrupting effect is tumor cell stress, downstream apoptosis, and tumor antigen release contributing as substrates for immunologic sensing. (FIG. 13B) Longer-term potential downregulation of charged glycans by tumor cells that may “escape” PMF-induced electromotive forces (EMFs): a key consequence of this may be loss of charged glycans (lower right scenario) that are essential for tumor cell repulsion, matrix migration, and repressive signals upon contact with nearby immune cells (e.g., Sia loss leading to a reduction in repressive Siglec-mediated signaling on NK cells and T cells).

FIGS. 14A-14C show pulsed magnetic field exposure inhibits growth of an immunotherapy sensitive lewis lung carcinoma (LLC) tumor line inoculated in the flanks of immunocompetent syngeneic (C57Bl/6) mice. (FIG. 14A) Beginning one week after LLC inoculation (2.5×10⁵ cells into hindquarter flank), growing tumors were subjected to 5 consecutive days of a daily 15 min exposure to a MagVenture rat cool-coil low frequency pulsed magnetic field (PMF) delivered in 15 Hz pulse trains at approximately 2 mm from the skin surface overlying the subcutaneous tumor. Mice were anesthetized for 15 min in both PMF and sham/control groups and controlled for sex (females, n=5 per group); graph of normalized tumor volumes at necropsy shows mean of controls versus that of the low frequency PMF (Lf-PMF) exposed group (**P<0.001 for difference in means). (FIG. 14B) To separately test effectiveness of an antibody to the T-cell inhibitory immune checkpoint receptor PD1 (“Anti-PD-1” antibody) in the system for such mice, a separate study was carried out to examine how an intraperitoneal dose (200 mcg) of anti-PD-1 antibody delivered every other day for the 7 day treatment period (beginning 1 week after LLC inoculation) might affect tumor volume. Graph shows means normalized to control for n=6 anti-PD-1 treated versus n=4 control females (*P<0.01 for difference). (FIG. 14C) To assess the effect of Lf-PMF exposure on a background of immune checkpoint (PD-1) blockade, among a total of n=10 female mice treated with 7 days of anti-PD-1 antibody delivered every other day beginning one week after LLC tumor inoculation, a subset (n=6) were exposed to an overlapping treatment with 5 days of daily Lf-PMF exposure under the same dose/schedule as in (A). Graph shows additional effect of Lf-PMF exposure on this background (**P<0.001 for difference in means).

### DETAILED DESCRIPTION

The disclosed method and compositions may be understood more readily by reference to the following detailed description of particular embodiments and the Examples included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. DEFINITIONS

It is understood that the disclosed method and compositions are not limited to the particular methodology, protocols, and reagents described as these may vary. It is also to be understood that the terminology used herein is for the purpose of describing particular embodiments only and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a nanoparticle” includes a single or a plurality of such nanoparticles, reference to “the nanoparticle” is a reference to one or more nanoparticles and equivalents thereof known to those skilled in the art, and so forth.

The word “or” as used herein means any one member of a particular list and also includes any combination of members of that list.

By a “therapeutically effective amount” of a composition as provided herein is meant a sufficient amount of the composition to provide the desired therapeutic effect. The exact amount required will vary from subject to subject, depending on the species, age, and general condition of the subject, the severity of disease (e.g., a neoplastic malignant cancer) that is being treated, the particular composition used, its mode of administration, and the like. Thus, it is not possible to specify an exact “therapeutically effective amount.” However, an appropriate “therapeutically effective amount” may be determined by one of ordinary skill in the art using only routine experimentation.

The term “therapeutic” refers to a composition that treats or ameliorates a symptom of a disease or disorder.

By “treat” is meant to administer a therapeutic or composition of the invention to a subject, such as a human or other mammal (for example, an animal model), that has an increased susceptibility for developing a disease or disorder (e.g. a neoplastic malignant cancer), or that has a disease or disorder (e.g. a neoplasm or a neoplastic malignant cancer), in order to prevent or delay a worsening of the effects of the disease or disorder, or to partially or fully reverse the effects of the disease or disorder.

By “prevent” is meant to minimize the chance that a subject who has an increased susceptibility for developing a disease or disorder (e.g., a neoplastic malignant cancer) or will end up with the disease or disorder (e.g., a neoplastic malignant cancer).

The terms “patient,” “subject,” “individual,” and the like are used interchangeably herein, and refer to any animal, or cells thereof whether in vitro or in situ, amenable to the methods described herein. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child, adolescent and newborn subjects, as well as fetuses, whether male or female, are intended to be covered. In some aspects, the subject has a neoplastic malignant cancer.

As used herein, “sample” is meant to mean an animal; a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

A “neoplastic malignant cancer” refers to a type of cancerous tumor that arises from abnormal cell growth (e.g., malignancy defined by tumors such as carcinomas, sarcomas, brain/gliomas, or mesotheliomas). A non-limiting list of sites where neoplastic malignant cancer can be treated with PMF include lung (small cell lung cancer or non-small cell lung cancer), thyroid, head or neck, nasopharynx, pharynx, nose or sinus, brain, spine, adrenal gland, pituitary gland, breast cancer, ovarian cancer, uterus, cervix, gastrointestinal (mouth, esophagus, stomach, duodenum, ileum, jejunum (small intestine), colon, rectum), lung, urogenital tract (uterus, ovary, cervix, endometrium, bladder, testis, penis, prostate), glia, hematology, endometrium, lymph, blood, muscle, skin (e.g., melanocytes), kidney, pancreas, liver, bone, bone marrow, Wilms tumor, or bile duct.

A “chemotherapy” refers to cancer treatment that uses one or more anti-cancer drugs. Types of chemotherapies include, but are not limited to, alkylating agents (e.g., cyclophosphamide, melphalan, chlorambucil, ifosfamide, busulfan. N-Nitroso-N-methylurea, carmustine, lomustine, semustine, fotemustine, streptozotocin, dacarbazine, mitozolomide, temozolomide, thiotepa, mitomycin, diaziquone, cisplatin, carboplatin, and oxaliplatin), antimetabolites (e.g., methotrexate), anti-microtubule agents (e.g., vinca alkaloids such as vincristine and taxanes such as paclitaxel and docetaxel), topoisomerase inhibitors (e.g., irinotecan and topotecan), and cytotoxic antibiotics (e.g., doxorubicin, pirarubicin, aclarubicin, mitoxantrone, actinomycin, bleomycin, and mitomycin).

An “immunotherapy” encompasses a diverse set of therapeutic strategies that harness or modify the immune system to prevent, control, or eliminate disease (e.g., cancer). Immunotherapies include monoclonal antibodies, cancer vaccines, immune checkpoint inhibitors, adoptive cell transfer, and cytokine therapies.

An “effective amount” of a compound is that amount of compound which is sufficient to provide a beneficial effect to the subject to which the compound is administered. The phrase “therapeutically effective amount”, as used herein, refers to an amount that is sufficient or effective to prevent or treat (delay or prevent the onset of, prevent the progression of, inhibit, decrease or reverse) a disease or condition, including alleviating symptoms of such diseases. An “effective amount” of a delivery vehicle is that amount sufficient to effectively bind or deliver a compound.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinence of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. METHODS

### 1. Glycocalyx

The glycocalyx is a microscopic, hair-like coating which covers the outer surface of virtually all cells, and plays a critical role in most signaling between cells and their surrounding environments. It is composed of branching biomolecules like proteins and lipids sometimes longer than the cell's own diameter, with various sugars covalently bound to them at particular locations.

The glycocalyx can also be known as the pericellular matrix and cell coat, is an external organelle consisting of a layer of glycosylated biomolecules called glycoconjugates, such as glycoproteins and glycolipids. These are embedded in and extend outwards from the cell membranes of virtually all cells. Generally, the carbohydrate portion of the glycolipids found on the surface of plasma membranes helps these molecules contribute to cell-cell recognition, communication, and intercellular adhesion.

The glycocalyx is a type of identifier that the body can use to distinguish between its own healthy cells and transplanted tissues, diseased cells, or invading organisms. Included in the glycocalyx are cell-adhesion molecules that enable cells to adhere to each other and guide the movement of cells during embryonic development. The glycocalyx plays a role in regulation of endothelial vascular tissue, including the modulation of red blood cell volume in capillaries.

A variety of cancers express unique glycocalyx that can have glycan modifications of tumor cell-surface proteins and lipids. A common modification is the upregulation of glycosaminoglycan (GAG) polymers, attached as sulfated glycans (heparan sulfate and chondroitin sulfate), or secreted into the immediate surround of the tumor cell as sulfated GAGs or (non-sulfated) hyaluronan chains (Chin-Hun Kuo et al 2018; Merry et al 2022). As a biophysical property distinct from the normal epithelial surround, such modifications endow tumor cells with substantial anionic surface charge. Moreover, sialic acid (Sia) monomer modifications of (typically heavy) tumor mucinous glycoproteins (Bellis et al 2022), which are also anionic at physiologic pH, contribute to what may be collectively considered a thick anionic glycocalyx “slab” over confluent carcinoma cell surfaces. This can contribute to shielding functions that physically block access of cross-reactive T cells to tumor-immunologic antigens on the tumor surface but also to cell-cell “repulsion” that facilitates tumor spread and invasion (Chin-Hun Kuo et al 2018; Fuster et al 2005).

The tumor cell surface can also serve specifically as an immunologic barrier. An example involves the overexpression of tumor Sia and repressive Siglec receptor-mediated signaling on NK or T cells attempting to engage with the tumor cell surface (Zheng et al 2020; Hudak et al 2014). The physical inability to make deeper contact with tumor surface antigens also imposes a kinetic inhibition in the ability of antigen-sensitized CD4 or CD8 cytotoxic T lymphocyte (CTL) cells to activate in response to shielded cognate antigens. With a glycocalyx layer rich in Sia, immunogenic epitopes may become “blocked” or shielded by the presence of heavy terminal glycan Sia modifications (Pinho et al 2015; Zheng et al 2020; Macauley et al 2014).

One kinetic argument regarding both naïve T cells and tumor antigen-sensitized cytotoxic T cells and NK cells in the TME is the inability to approach or “remain near” tumor antigen sources or tumor-cell nests in the tumor mass. Such nests are often “islands” among a stromal cell surround, and the additional glycocalyx shielding may isolate potential anti-tumor T cells and NK cells from tumor cell nests, despite chemokine and cytokine signals that may typically promote migration and improved proximity. Such stromal cells, including cancer-associated fibroblasts and remodeling endothelium, can promote tumor a pre-invasive phenotype. This occurs because of the ability of such stromal cells to promote pro-metastatic conditions and a TME that suppresses anti-tumor immunity, although the behavior of stromal cells can also vary with TME spatial distribution (Zhao et al 2023).

The tumor ECM is often rich in dense networks of highly enriched secreted molecules that can further shield tumor cells from immune cells in the TME. These networks are often rich in the same anionic molecules (e.g., glycosaminoglycans on secreted proteoglycan core proteins) that promote attachment of growth factors via basic amino acid-rich domains in the form of “banks” for advancing tumor cells or remodeling vasculature (Yang et al 2023; Vlodavsky et al 2023). Enzymes such as heparinase or hyaluronidase released by such cells may also mobilize such factors in the promotion of tumor growth, while the tumor ECM barrier and interstitium expand the barrier to surrounding immunologic cells.

The tortuous and leaky vascular supply of tumors, which expands through the action of a variety of pro-angiogenic tumor growth factors (e.g., VEGF mitogen family, FGFs, and PDGF) (Liu et al 2023), is relatively inefficient in extending capillary beds throughout tumor nests, often reaching an extreme in central hypoxic tumor zones that become necrotic. This can limit access to anti-tumor agents (molecular or cell therapies) to the tumor mass as well as endogenous colonization by circulating anti-tumor T cells or NK cells. Tumor vascular proliferation under tumor VEGF stimulation is not only tortuous but also leaky, with a net effect of increasing intra-tumor interstitial pressure (Qian et al 2023), which further limits the ability of systemic or tumor-peripheral immune cells (including naïve or sensitized cytotoxic T cells) to access the inner depths of the tumor (Qian et al 2023; Kalli et al 2024).

Tumor lymphatic vasculature may serve as a means for naïve and sensitized T cells to access regions of the tumor that may be susceptible to immunologic attack and release of tumor antigens. The latter, including antigens from metastatic or dying tumor cells (possibly boosted by cytotoxic therapies), may flow to regional lymph nodes via draining lymphatic vessels to regional lymph nodes, or even tertiary lymphoid structures (TLSs) along lung bronchovasculature, providing antigen substrate for APC uptake and cross-presentation with immune activation and proliferation of anti-tumor cytotoxic T cells (Preet Kaur et al 2023). Tumor dendritic cells may also traffic in the same direction, carrying tumor antigen for presentation to draining nodes.

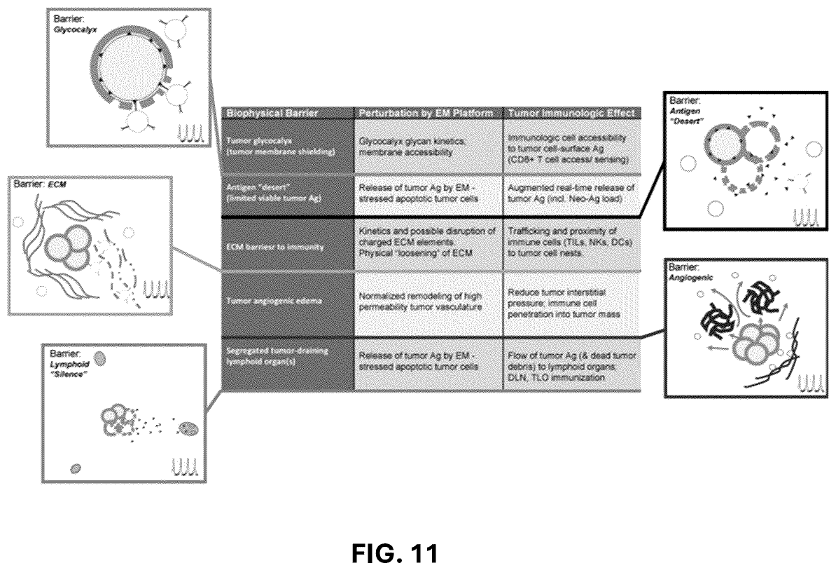

FIG. 11 shows a variety of physical and immunologic barriers to anti-tumor therapeutic approaches, with a focus on how EM platforms may directly or indirectly augment “access” to antitumor cellular immunity through biophysical EM effector mechanisms that address each barrier (summarized in the table at center).

As used herein, the term “non-malignant glycocalyx” refers to the glycocalyx (hydrated cell-surface complex carbohydrate layer) that covers the plasma membrane of normal cells (e.g. non-malignant (cancer) cells). A non-malignant glycocalyx expresses relatively low levels of glycocalyx sialic acids and sulfated glycosaminoglycans (compared to that of malignant glycocalyx expression on cancer cells).

While certain tumors overexpress distinct proteoglycans into the tumor glycocalyx, others over-express heavy Sia modifications on glycoproteins or polysialic acid (polySia) chains extended on glycan termini decorating unique tumor core proteins.

As used herein, the term “malignant glycocalyx A” refers to an abnormal glycocalyx of malignant (cancer) cells that is unlikely to engage with low frequency (<300 Hz) pulsed magnetic fields (PMFs). A malignant glycocalyx A has a density of anionic glycan charge that is too low to engage with induced electromotive forces that drive cancer membrane stress and altered signaling. A malignant glycocalyx A has a low- or absent expression of glycocalyx sialic acids and sulfated glycosaminoglycans as determined by flow cytometry. Furthermore, malignant glycocalyx A is less likely to be “primed” or remodeled with repeated PMF exposures.

As used herein, the term “malignant glycocalyx B” refers to an abnormal glycocalyx of malignant (cancer) cells that has a high probability of engaging with low frequency pulsed magnetic fields. A malignant glycocalyx B has a density of anionic glycan charge that is sufficient to engage with induced electromotive forces that drive cancer membrane stress and altered signaling. A malignant glycocalyx B has a high expression of either glycocalyx sialic acids or sulfated glycosaminoglycans as determined by flow cytometry. Furthermore, a malignant glycocalyx B will likely be “primed,” and remodel in response to repeated PMF exposures.

In some aspects, to determine whether a cell has a non-malignant glycocalyx, a malignant glycocalyx A, or a malignant glycocalyx B, cells from a biopsy can be digested into a single-cell suspension, and tagged with fluorescent lectin and fluorescent FGF-2 probes that detect intensity of glycocalyx sialic acids and sulfated glycosaminoglycans, respectively by flow cytometry: Non-malignant cells would express relatively low levels of glycocalyx sialic acids and sulfated glycosaminoglycans (compared to that of malignant glycocalyx expression on cancer cells). Low- or absent expression of glycocalyx sialic acids and sulfated glycosaminoglycans by flow cytometry would define “Glycocalyx A” tumor cells (with biopsy containing confirmed tumor cells in the specimen). High expression of either glycocalyx sialic acids or sulfated glycosaminoglycans by flow cytometry would define “Glycocalyx B” tumor cells (with biopsy containing confirmed tumor cells in the specimen).

### 2. Pulsed Magnetic Field(s)

Disclosed are methods comprising, in part, applying a pulsed magnetic field (PMF) to a subject, a cell, or a population of cells.

There is a rationale for integrating anti-tumor immunity strategies with electromagnetic (EM) platforms. Any number of the above exposures may lead to conditions that promote anti-cancer immunity, with an opportunity to apply such unique physical states to tumor cells as novel “substrates” for mechanisms that boost anti-tumor immunity. The paradigm presented herein examines how biophysical parameters of EM waves or pulse sequences driven by EM wave-generating or magnetic sources may modulate a cancer cell population and/or a tumor microenvironment (TME) to promote conditions that facilitate anti-tumor T-cell immunity and in some cases even humoral immunity, as it involves distinct classes of immune cells. A major focus is the optimization of states that promote the endogenous tumor-specific immune synapse (Carrasco-Padilla et al 2002). This includes augmented driving of immunologic signal-1 events (i.e., antigen-driven T-cell engagement as the “first signal”) to maintain anti-tumor immune specificity and efficacy in the setting of an unstable and pleomorphic carcinoma-cell landscape.

An electromagnetic wave is a traveling wave composed of electric (E) and magnetic (B) field vectors that are orthogonal to each other and classically travel through space in a direction perpendicular to the E and B vectors, with the power of the EM wave defined as the Poynting vector (cross-product of the maximum E- and B-field components).

Such waves have often been tested in the radiofrequency range, which can penetrate tissue to some extent (RF “skin depth”, limited by tissue dielectric properties) and, with enough power, can induce thermal effects. An electric field applied to a tissue surface generally attenuates greatly upon entry from atmospheric space, with greater tissue depth at lower frequencies (e.g., plane wave distances on the order of centimeters in muscle in the MHz-GHz range) (Polk et al 1995). A variety of frequencies from the extremely low frequency (ELF; <300 Hz) to radiofrequency (kHz-MHz) range have been used to alter the growth and/or apoptosis of cultured tumor cells at energy levels that do not necessarily induce thermal effects, particularly with EM platforms used to generate pulsed magnetic fields at frequencies under 100 kHz (Xu et al 2021; Tofani et al 2022). At greater frequencies (GHz-THz), low energy (non-thermal) delivery modes can be applied, but tissue attenuation may be greater, and data do not necessarily reveal greater anti-tumor effects at higher frequencies (Xu et al 2021; Tofani et al 2022). Nevertheless, higher ranges (gamma or X-ray) result in significant ionizing potential. The focus herein is on non-thermal and non-ionizing EM platforms in an attempt to minimize tissue damage, including any harmful effects on bystander immune cells, including T cells that maybe sensitized or recruited if naïve within a tumor microenvironment.

Disclosed herein are methods of applying a magnetic field, either statically or in oscillating or pulsed formats (Xu et al 2021). If the B-field is pulsed or oscillates, this can generate induced electromotive forces (EMFs) by Faraday's law operating within the tissue through which the B-field fluxes (Gaynor et al 2018). The induced EMF would then be able to drive or force any charged mass (e.g., anionic glycans in the tissue or tumor membranes) in the tissue. The flux operates across any theoretical conductor of charge deep in the tissue defined by the product of dB/dt (where the B-field changes from min to max over interval time “dt”) and the area (A) bounded by the “circuit” perimeter over which the induced potential/EMF operates (in this case, where the B-field moves perpendicular through area A bounded by the EMF induced along the perimeter of A). The oscillations or pulsations may be pulsed over any arbitrary “duty cycle”, even over a very narrow “rise time”, allowing for relatively low-magnitude B-fields to generate appreciable dB/dt over a reasonably small cross-sectional area, A, through which the field fluxes to generate the EMF along the perimeter of A.

Engaging such fields physically with cellular elements or characteristics that are unique to tumor cells becomes functionally interesting while ensuring that normal cells remain intact/unaffected. Targeting or physically engaging with a charged or ion-dense cancer cell-specific component with any EM-wave or B-field induction approach is a central interest, disrupting a tumor cellular structure or process that ultimately i) ablates the tumor cell or ii) leads to greater exposure of local immunity to tumor antigens. While these “cytotoxic” approaches would ideally fully spare normal surrounding stromal cells/tissues, one may consider approaches that achieve a greater probability or magnitude of detrimental effects (e.g., immediate or delayed cell death) in cancer cells than that of surrounding host cells. The latter may simply express a lesser degree of unique targets (e.g., charge density) that make the cancer cell vulnerable to the EM platform. Such features include charged elements for cell proliferation, energetics, or redox homeostasis (e.g., S-phase chromatin/DNA, microtubule elements, mitochondrial membrane, or downstream free-radical oxygen species) or charged glycans that are integral to the tumor membrane glycocalyx (e.g., glycosaminoglycans and/or sialic acid modified glycoproteins) (Zadeh-Haghighi et al 2022; Wang et al 2017; Chin-Hun Kuo et al 2018; Ashdown et al 2020; Pinho et al 2015; Paszek et al 2014; Johns et al 2023). Electric conduction via a highly anionic tumor-membrane contiguous glycocalyx or tumor cell-cell junctions may permit lesser resistance for electric conduction of induced EMFs. At the quantum level, electron spin pairs may be uniquely susceptible to pulsed magnetic fields, which can impact biological phenomena at energies below the thermal background (Zadeh-Haghighi et al 2022; Hore et al 2016). Further, pulsed electric field (PEF) delivery probes may be used to contact a tumor border directly, inducing membrane stress and even cancer-cell pore induction while taking advantage of unique tumor-conducting properties. Electroporation of tumor cells with its associated stress and tumor-cell death is a major mechanism in such applications (Batista Napotnik et al 2021).

Apoptotic and growth-inhibitory tumor cell responses appear to evolve secondary to many of these perturbations (Xu et al 2021; Tofani et al 2022). Potentially, a variety of “upstream” impacts from EM field exposures could evolve to drive apoptosis, including EM-driven cell membrane nanopore formation and associated cell stress, integrin-associated downstream focal adhesion and cytoskeletal signaling alterations, reactive oxygen species (ROS) generation with mitochondrial-driven apoptotic pathway activation, and possibly other pathways involving microtubular effects or effects on cyclin-dependent kinases that impact cell division (Xu et al 2021; Wang et al 2017; Johns et al 2023). In addition to apoptosis and the above considerations, a variety of secondary effects on cell growth-signaling and/or survival-signaling pathways may theoretically also contribute to tumor growth inhibition following EM exposure.

As used herein, the term “pulsed magnetic field” (PMF) or pulsed electromagnetic field are electromagnetic fields that vary over time. Natural phenomena such as lightning bolts may generate PMF. These naturally-occurring PMF create a global electromagnetic resonance within the electromagnetic field of the earth. These resonances arise due to the waveguide characteristics of the Earth's ionosphere. These resonances are called Schumann resonances after the physicist who discovered their existence.

In various aspects of the disclosed methods, the methods comprise administering a pulsed electromagnetic field to a subject. In some aspects, the frequency of the pulsed electromagnetic field can be about 50-385 Hz.

In some aspects, the intensity of the pulsed electromagnetic field that is administered to the subject is about 5-30 milliTesla (mT). The actual intensity of the pulsed electromagnetic field used depends on the type of tumor or tumor cell being treated and the location of the tumor within the body. In some aspects, the pulsed electromagnetic field is administered at a 20 mT maximum field (dB/dt˜2 T/sec).

In some aspects, the total exposure time of a subject to the pulsed magnetic field can range from about 1 minute to about 60 minutes in each therapy session.

As disclosed herein, pulsed magnetic fields can induce electromotive forces with unique induced voltage profiles across deep tissue. This may drive movement of charge deep in the tissue and possibly with a distinct distribution of uniform induced-EMF amplitudes across a deep-seated tumor thickness. Effects resulting from such a classical application of Faraday's law of induction using pulsed magnetic fields (PMFs) are compelling and reasonably may operate to engage charge over a fairly wide spatial area (over contiguous tumor cells in a small/growing tumor, for example) exposed to either radiofrequency EM waves or pulsed magnetic fields (Xu et al 2021; Mills et al 2000; Polk et al 1995; Pall et al 2022).

In some aspects of the methods disclosed herein, a low-frequency PMF that effectively couples (e.g., with substantial mechanical response) to a unique tumor cellular feature such as a highly charged tumor glycocalyx may “oscillate” at either 10 Hz or 10 kHz; however, the duty cycle and dB/dt may be designed identically for these two frequencies (for example, dB/dt delivered as 10 μT over 20 μs in both cases), wherein the efficacy can lie in the duty cycle rise time over 20 μs rather than the PMF frequency (10 Hz or 10 kHz) per se. Thus, in some aspects, a relatively low pulse amplitudes may be used to affect tumor-characteristic properties so long as “dt” is very short (i.e., narrow pulse width), and thus the independent parameters of dBmax (amplitude) and “dt” (effectively, the period of the duty cycle) should be separately reported. In some aspects, independent of frequency, if the period of a pulsed 50 mT B-field can deliver pulses over a 50-μs duty cycle (i.e., rise time <50 μs), the reporting of “a PMF delivered at dB/dt=1.0 kT/s” without information on the pulse-width parameters does not provide sufficient information. A PMF of 500 mT delivered over 500 μs could also be reported as “1.0 kT/s”, although such an exposure may be different than the former (possibly with markedly different biological effects), given the pulse delivery over a 10-fold narrower duty cycle in the former.

### 3. Methods of Treatment of a Neoplastic Malignant Cell Cancer

Disclosed are methods of treating a neoplastic malignant cell cancer in a subject in need thereof comprising: a) obtaining or having obtained a sample from the subject, wherein the sample comprises at least one neoplastic malignant cancer cell; b) determining that the isolated neoplastic malignant cancer cell comprises a malignant glycocalyx B; and c) applying a pulsed magnetic field (PMF) to the subject, thereby treating the neoplastic malignant cell cancer in the subject.

Also disclosed are methods of treating a neoplastic malignant cell cancer in a subject in need thereof comprising: applying a pulsed magnetic field (PMF) to the subject, wherein the subject has a malignant glycocalyx B, thereby treating the neoplastic malignant cell cancer in the subject. A method of treating a neoplastic malignant cell cancer in a subject in need thereof comprising: applying a pulsed magnetic field (PMF) to the subject, wherein the subject has a malignant glycocalyx B and not a malignant glycocalyx A, thereby treating the neoplastic malignant cell cancer in the subject.

In some aspects of the methods of treating a neoplastic malignant cell cancer, the method further comprises administering a therapeutically effective amount an immunotherapy to the subject. In some aspects of the methods of treating a neoplastic malignant cell cancer, the therapeutically effective amount the immunotherapy is administered before, during, and/or after applying the pulsed magnetic field to the subject.

As used herein, a “malignant neoplasm” refers to a cancerous tumor. The term “neoplasm” refers to an abnormal growth of tissue. The term “malignant” means the tumor is cancerous and is likely to spread (metastasize) beyond its point of origin. Examples of malignant neoplasms include, but are not limited to carcinomas, sarcomas, myelomas, leukemias and lymphomas.

Disclosed are methods of treating a malignant neoplasm in a subject in need thereof comprising: a) obtaining or having obtained a sample from the subject, wherein the sample comprises at least one neoplastic malignant cancer cell; b) determining that the isolated neoplastic malignant cancer cell comprises a malignant glycocalyx B; and c) applying a pulsed magnetic field (PMF) to the subject, thereby treating the malignant neoplasm in the subject.

In some aspects, the methods of treating a neoplastic malignant cell cancer or methods of treating a malignant neoplasm can further comprise administering one or more immunotherapies to a subject. In some aspects, the immunotherapy can be a checkpoint inhibitor. In some aspects, the terms “checkpoint inhibitors” and “immune checkpoint inhibitors” can be used interchangeably.

The methods disclosed herein comprise administering one or more checkpoint inhibitors to a subject. In some aspects, the checkpoint inhibitor can block CTLA-4 (cytotoxic T lymphocyte associated protein 4), PD-1 (programmed cell death protein 1) or PD-L1 (programmed cell death ligand 1).

In some aspects, the checkpoint inhibitor can be, but is not limited to, ipilimumab (Yervoy), pembrolizumab (Keytruda), nivolumab (Opdivo), cemiplimab (trade name Libtayo), and dostarlimab (Jemperli), atezolizumab (Tecentriq), durvalumab (Imfinzi), or avelumab (Bavencio), or a combination thereof. In some aspects, the checkpoint inhibitor can be, but is not limited to, Tremelimumab, Sintilimab (formerly IBI308; Tyvyt), Tislelizumab (formerly BGB-A317), Toripalimab (formerly JS 001), Spartalizumab (formerly PRD001); Camrelizumab (formerly SHR1210), KN035, Cosibelimab (formerly CK-301), CA-170, or BMS-986189, or a combination thereof.

In such embodiments the checkpoint inhibitor can be an inhibitor that blocks CTLA-4 (cytotoxic T lymphocyte associated protein 4), PD-1 (programmed cell death protein 1) or PD-L1 (programmed cell death ligand 1). In some aspects, the checkpoint inhibitor previously administered to the subject can be, but is not limited to, ipilimumab (Yervoy), pembrolizumab (Keytruda), nivolumab (Opdivo), cemiplimab (trade name Libtayo), and dostarlimab (Jemperli), atezolizumab (Tecentriq), durvalumab (Imfinzi), or avelumab (Bavencio), or a combination thereof. In some aspects, the checkpoint inhibitor can be, but is not limited to, tremelimumab, sintilimab (formerly IBI308; tyvyt), tislelizumab (formerly BGB-A317), toripalimab (formerly JS 001), spartalizumab (formerly PRD001); camrelizumab (formerly SHR1210), KN035, cosibelimab (formerly CK-301), CA-170, or BMS-986189, or a combination thereof.

In some aspects, the checkpoint inhibitor is a PD-1 inhibitor, PD-L1 inhibitor, or CTLA4 inhibitor. In some aspects, a PD-1 inhibitor can be, but is not limited to, Nivolumab, Pembrolizumab, or Cemiplimab. In some aspects, a PD-L1 inhibitor can be, but is not limited to, Atezolizumab, Avelumab, or Durvalumab. In some aspects, a CTLA-4 inhibitor can be, but is not limited to, ipilimumab or tremelimumab.

In some aspects, the methods of treating a neoplastic malignant cell cancer or methods of treating a malignant neoplasm can further comprise administering one or more chemotherapies to a subject. In some aspects, the chemotherapy can be an alkylating agent, plant alkaloid, antimetabolite, anthracycline, topoisomerase inhibitor or corticosteroid.

In some aspects of the methods of treating a neoplastic malignant cell cancer, the method further comprises administering a therapeutically effective amount of chemotherapy to the subject. In some aspects of the methods of treating a neoplastic malignant cell cancer, the therapeutically effective amount the chemotherapy is administered before, during, and/or after applying the pulsed magnetic field to the subject.

In some aspects, a chemotherapy can be, but is not limited to, an alkylating agent, an antimetabolite agent, an antineoplastic antibiotic agent, a mitotic inhibitor agent.

In some aspects, the chemotherapy can be, but is not limited to, docetaxel, carboplatin, cisplatin, oxaliplatin, Dacarbazine, procarbazine, temozolomide, Busulfan, Lomustine, bendamustine, cyclophosphamide, ifosfamide, Fluorouracil, Gemcitabine, Doxorubicin, Methotrexate, Capecitabine, Altretamine, Epirubicin, Irinotecan, Daunorubicin, Paclitaxel, Chlorambucil, Idarubicin, Cabazitaxel, Floxuridine, Pembrolizumab, Nivolumab, durvalumab, Cemiplimab, Ipilimumab, atezolizumab, Niraparib, Olaparib, Bevacizumab, alpelisib, Nab-paclitaxel (Abraxane), Sacituzumab govitecan, Dato-Dxd, taxol, Lenvatinib, everolimus, Letrozole, Gefitinib, Erlotinib, or sorafenib.

In some aspects, the chemotherapy can be one or more of Altretamine, Bendamustine, Busulfan, Carboplatin, Chlorambucil, Cisplatin, Cyclophosphamide, Dacarbazine, Ifosfamide, Mechlorethamine, Melphalan, Oxaliplatin, Procarbazine, Temozolomide, Thiotepa, Trabectedin, Carmustine, Lomustine, Streptozocin, 5-fluorouracil, 6-mercaptopurine, Azacitidine, Capecitabine, Cladribine, Clofarabine, Cytarabine, Decitabine, Floxuridine, Fludarabine, Gemcitabine, Hydroxyurea, Methotrexate, Nelarabine, Pemetrexed, Pentostatin, Pralatrexate, Thioguanine, Trifluridine/tipiracil combination, Etoposide, Irinotecan, Irinotecan liposomal, Mitoxantrone, Teniposide, Topotecan, Cabazitaxel, Docetaxel, Nab-paclitaxel, Paclitaxel, Vinblastine, Vincristine, Vincristine liposomal, Vinorelbine, Daunorubicin, Doxorubicin, Doxorubicin liposomal, Epirubicin, Idarubicin, Mitoxantrone, Valrubicin, Bleomycin, Dactinomycin, Mitomycin-C, all-trans-retinoic acid, Arsenic trioxide, Asparaginase, Eribulin, Ixabepilone, Mitotane, Omacetaxine, Pegaspargase, Procarbazine, Romidepsin, Vorinostat, Dexamethasone, Hydrocortisone, Methylprednisolone, Prednisolone, or Prednisone.

In some aspects of the methods of treating a neoplastic malignant cell cancer, the PMF is administered daily for about 7 days. In some aspects of the methods of treating a neoplastic malignant cell cancer, the PMF is administered daily for about 7 days, about 10 days, about 12 days, about 14 days, or about 21 days. In some aspects of the methods of treating a neoplastic malignant cell cancer, the PMF is administered daily for about 7 days during each round of immunotherapy. In some aspects of the methods of treating a neoplastic malignant cell cancer, the PMF is administered for about 7 days, about 10 days, about 12 days, about 14 days, or about 21 days during each round of immunotherapy.

In some aspects of the methods of treating a neoplastic malignant cell cancer, the PMF is administered at an amplitude of about 5-30 mT. In some aspects of the methods of treating a neoplastic malignant cell cancer, the PMF is administered at an amplitude of about 10-20 mT or about 15-20 mT, or at an amplitude of about 5 mT, 10 mT, 15 mT, 20 mT, 25 mT or 30 mT. In some aspects of the methods of treating a neoplastic malignant cell cancer, the PMF is administered at a 20 mT maximum field (dB/dt˜2 T/sec).

In some aspects of the methods of treating a neoplastic malignant cell cancer, the PMF is administered at a frequency of about 50 Hz to 385 Hz. In some aspects of the methods of treating a neoplastic malignant cell cancer, the PMF is administered at a frequency of about 100 Hz to 385 Hz, 150 Hz to 350 Hz, 200 Hz to 300 Hz, or at the frequency of about 50 Hz, 100 Hz, 150 Hz, 200 Hz, 250 Hz, 300 Hz, 350 Hz, or 385 Hz.

In some aspects of the methods of treating a neoplastic malignant cell cancer, the PMF is 20 mT maximum field (dB/dt˜2 T/sec) over a 10 msec duty cycle at 50 Hz.

In some aspects of the methods of treating a neoplastic malignant cell cancer, the PMF is administered at a duty-cycle pulse width of 70 μsec using 15 Hz pulse trains.

In some aspects of the methods of treating a neoplastic malignant cell cancer, the PMF is administered at a pulse duty-cycle rise time of about 5-15 msec. In some aspects of the methods of treating a neoplastic malignant cell cancer, the PMF is administered at a pulse duty-cycle rise time of about 5, 6, 7, 8, 9, 10, 11, 12, 13, 14, or 15 msec.

In some aspects of the methods of treating a neoplastic malignant cell cancer, the method further comprises administering radiation therapy to the subject. In some aspects of the methods of treating a neoplastic malignant cell cancer, the radiation therapy is administered before, during, and/or after applying the alternating electric field.

In some aspects of the methods of treating a neoplastic malignant cell cancer, the isolated neoplastic malignant cancer cell does not comprise a malignant glycocalyx A.

In some aspects of the methods of treating a neoplastic malignant cell cancer, the neoplastic malignant cell cancer is a carcinoma, a sarcoma, a myeloma, a leukemia, or a lymphoma. In some aspects of the methods of treating a neoplastic malignant cell cancer, the neoplastic malignant cell cancer is skin cancer, breast cancer, prostate cancer, bladder cancer, cervical cancer, endometrial cancer, lung cancer, colon cancer, or rectal cancer. In some aspects of the methods of treating a neoplastic malignant cell cancer, the neoplastic malignant cell cancer is brain cancer. In some aspects of the methods of treating a neoplastic malignant cell cancer, the neoplastic malignant cell cancer is lung cancer.

In some aspects of the methods of treating a neoplastic malignant cell cancer, the pulsed magnetic field is administered for about 1 minute to about 60 minutes per session. In some aspects of the methods of treating a neoplastic malignant cell cancer, the pulsed magnetic field is administered for about 1, 5, 10, 15, 20, 25, 30, 35, 40, 45, 50, 55, or 60 minutes.

### 4. Methods of Inducing Apoptosis of One or More Neoplastic Malignant Cells

Disclosed are methods of inducing apoptosis of one or more neoplastic malignant cells comprising: a) determining that the one or more neoplastic malignant cells comprises a malignant glycocalyx B; and b) applying a pulsed magnetic field (PMF) to the one or more neoplastic malignant cells.

In some aspects of the methods of inducing apoptosis of one or more neoplastic malignant cells, the one or more neoplastic malignant cells are in culture. In some aspects of the methods of inducing apoptosis of one or more neoplastic malignant cells, the one or more neoplastic malignant cells are in a subject.

In some aspects of the methods of inducing apoptosis of one or more neoplastic malignant cells, the method further comprises exposing the one or more neoplastic malignant cells to a therapeutically effective amount an immunotherapy. In some aspects of the methods of inducing apoptosis of one or more neoplastic malignant cells, the one or more neoplastic malignant cells are exposed to the therapeutically effective amount the immunotherapy before, during, and/or after applying the pulsed magnetic field to the one or more neoplastic malignant cells.

In some aspects of the methods of inducing apoptosis of one or more neoplastic malignant cells, the method further comprises exposing the one or more neoplastic malignant cells to a therapeutically effective amount a chemotherapy. In some aspects of the methods of inducing apoptosis of one or more neoplastic malignant cells, the one or more neoplastic malignant cells are exposed to the therapeutically effective amount the chemotherapy before, during, and/or after applying the pulsed magnetic field to the one or more neoplastic malignant cells.

In some aspects, the methods of inducing apoptosis of one or more neoplastic malignant cells can further comprise exposing the one or more neoplastic malignant cells to an immunotherapy.

In some aspects, the immunotherapy can be a checkpoint inhibitor. In some aspects, the terms “checkpoint inhibitors” and “immune checkpoint inhibitors” can be used interchangeably.

The methods disclosed herein comprise administering one or more checkpoint inhibitors to a subject. In some aspects, the checkpoint inhibitor can block CTLA-4 (cytotoxic T lymphocyte associated protein 4), PD-1 (programmed cell death protein 1) or PD-L1 (programmed cell death ligand 1).

In some aspects, the checkpoint inhibitor can be, but is not limited to, ipilimumab (Yervoy), pembrolizumab (Keytruda), nivolumab (Opdivo), cemiplimab (trade name Libtayo), and dostarlimab (Jemperli), atezolizumab (Tecentriq), durvalumab (Imfinzi), or avelumab (Bavencio), or a combination thereof. In some aspects, the checkpoint inhibitor can be, but is not limited to, Tremelimumab, Sintilimab (formerly IBI308; Tyvyt), Tislelizumab (formerly BGB-A317), Toripalimab (formerly JS 001), Spartalizumab (formerly PRD001); Camrelizumab (formerly SHR1210), KN035, Cosibelimab (formerly CK-301), CA-170, or BMS-986189, or a combination thereof.

In such embodiments the checkpoint inhibitor can be an inhibitor that blocks CTLA-4 (cytotoxic T lymphocyte associated protein 4), PD-1 (programmed cell death protein 1) or PD-L1 (programmed cell death ligand 1). In some aspects, the checkpoint inhibitor previously administered to the subject can be, but is not limited to, ipilimumab (Yervoy), pembrolizumab (Keytruda), nivolumab (Opdivo), cemiplimab (trade name Libtayo), and dostarlimab (Jemperli), atezolizumab (Tecentriq), durvalumab (Imfinzi), or avelumab (Bavencio), or a combination thereof. In some aspects, the checkpoint inhibitor can be, but is not limited to, tremelimumab, sintilimab (formerly IBI308; tyvyt), tislelizumab (formerly BGB-A317), toripalimab (formerly JS 001), spartalizumab (formerly PRD001); camrelizumab (formerly SHR1210), KN035, cosibelimab (formerly CK-301), CA-170, or BMS-986189, or a combination thereof.

In some aspects, the checkpoint inhibitor is a PD-1 inhibitor, PD-L1 inhibitor, or CTLA4 inhibitor. In some aspects, a PD-1 inhibitor can be, but is not limited to, Nivolumab, Pembrolizumab, or Cemiplimab. In some aspects, a PD-L1 inhibitor can be, but is not limited to, Atezolizumab, Avelumab, or Durvalumab. In some aspects, a CTLA-4 inhibitor can be, but is not limited to, ipilimumab or tremelimumab.

In some aspects, the methods of inducing apoptosis of one or more neoplastic malignant cells can further comprise exposing the one or more neoplastic malignant cells to a chempotherapy. In some aspects, the chemotherapy can be an alkylating agent, plant alkaloid, antimetabolite, anthracycline, topoisomerase inhibitor or corticosteroid.

In some aspects of the methods of treating a neoplastic malignant cell cancer, the method further comprises administering a therapeutically effective amount of chemotherapy to the subject. In some aspects of the methods of treating a neoplastic malignant cell cancer, the therapeutically effective amount the chemotherapy is administered before, during, and/or after applying the pulsed magnetic field to the subject.

In some aspects, a chemotherapy can be, but is not limited to, an alkylating agent, an antimetabolite agent, an antineoplastic antibiotic agent, a mitotic inhibitor agent.

In some aspects, the chemotherapy can be, but is not limited to, docetaxel, carboplatin, cisplatin, oxaliplatin, Dacarbazine, procarbazine, temozolomide, Busulfan, Lomustine, bendamustine, cyclophosphamide, ifosfamide, Fluorouracil, Gemcitabine, Doxorubicin, Methotrexate, Capecitabine, Altretamine, Epirubicin, Irinotecan, Daunorubicin, Paclitaxel, Chlorambucil, Idarubicin, Cabazitaxel, Floxuridine, Pembrolizumab, Nivolumab, durvalumab, Cemiplimab, Ipilimumab, atezolizumab, Niraparib, Olaparib, Bevacizumab, alpelisib, Nab-paclitaxel (Abraxane), Sacituzumab govitecan, Dato-Dxd, taxol, Lenvatinib, everolimus, Letrozole, Gefitinib, Erlotinib, or sorafenib.

In some aspects, the chemotherapy can be one or more of Altretamine, Bendamustine, Busulfan, Carboplatin, Chlorambucil, Cisplatin, Cyclophosphamide, Dacarbazine, Ifosfamide, Mechlorethamine, Melphalan, Oxaliplatin, Procarbazine, Temozolomide, Thiotepa, Trabectedin, Carmustine, Lomustine, Streptozocin, 5-fluorouracil, 6-mercaptopurine, Azacitidine, Capecitabine, Cladribine, Clofarabine, Cytarabine, Decitabine, Floxuridine, Fludarabine, Gemcitabine, Hydroxyurea, Methotrexate, Nelarabine, Pemetrexed, Pentostatin, Pralatrexate, Thioguanine, Trifluridine/tipiracil combination, Etoposide, Irinotecan, Irinotecan liposomal, Mitoxantrone, Teniposide, Topotecan, Cabazitaxel, Docetaxel, Nab-paclitaxel, Paclitaxel, Vinblastine, Vincristine, Vincristine liposomal, Vinorelbine, Daunorubicin, Doxorubicin, Doxorubicin liposomal, Epirubicin, Idarubicin, Mitoxantrone, Valrubicin, Bleomycin, Dactinomycin, Mitomycin-C, all-trans-retinoic acid, Arsenic trioxide, Asparaginase, Eribulin, Ixabepilone, Mitotane, Omacetaxine, Pegaspargase, Procarbazine, Romidepsin, Vorinostat, Dexamethasone, Hydrocortisone, Methylprednisolone, Prednisolone, or Prednisone.

In some aspects of the methods of inducing apoptosis of one or more neoplastic malignant cells, the PMF is applied daily for about 7 days. In some aspects of the methods of inducing apoptosis of one or more neoplastic malignant cells, the PMF is administered daily for about 7 days, about 10 days, about 12 days, about 14 days, or about 21 days. In some aspects of the methods of inducing apoptosis of one or more neoplastic malignant cells, the PMF is applied daily for about 7 days during each round of immunotherapy. In some aspects of the methods of inducing apoptosis of one or more neoplastic malignant cells, the PMF is administered for about 7 days, about 10 days, about 12 days, about 14 days, or about 21 days during each round of immunotherapy.

In some aspects of the methods of inducing apoptosis of one or more neoplastic malignant cells, the PMF is administered at an amplitude of about 5-30 mT. In some aspects of the methods of inducing apoptosis of one or more neoplastic malignant cells, the PMF is administered at an amplitude of about 10-20 mT or about 15-20 mT, or at an amplitude of about 5 mT, 10 mT, 15 mT, 20 mT, 25 mT or 30 mT. In some aspects of the methods of inducing apoptosis of one or more neoplastic malignant cells, the PMF is administered at a 20 mT maximum field (dB/dt˜2 T/sec).

In some aspects of the methods of inducing apoptosis of one or more neoplastic malignant cells, the PMF is administered at a frequency of about 50 Hz to 385 Hz. In some aspects of the methods of inducing apoptosis of one or more neoplastic malignant cells, the PMF is administered at a frequency of about 100 Hz to 385 Hz, 150 Hz to 350 Hz, 200 Hz to 300 Hz, or at the frequency of about 50 Hz, 100 Hz, 150 Hz, 200 Hz, 250 Hz, 300 Hz, 350 Hz, or 385 Hz.

In some aspects of the methods of inducing apoptosis of one or more neoplastic malignant cells, the PMF is 20 mT maximum field (dB/dt˜2 T/sec) over a 10 msec duty cycle at 50 Hz.

In some aspects of the methods of inducing apoptosis of one or more neoplastic malignant cells, the PMF is administered at a duty-cycle pulse width of 70 μsec using 15 Hz pulse trains. In some aspects of the methods of inducing apoptosis of one or more neoplastic malignant cells, the PMF is administered at a pulse duty-cycle rise time of about 5, 6, 7, 8, 9, 10, 11, 12, 13, 14, or 15 msec.

In some aspects of the methods of inducing apoptosis of one or more neoplastic malignant cells, the PMF is administered at a pulse duty-cycle rise time of about 5-15 msec.

In some aspects of the methods of inducing apoptosis of one or more neoplastic malignant cells, the method further comprises exposing the one or more neoplastic malignant cells to a therapeutically effective amount a radiation therapy. In some aspects of the methods of inducing apoptosis of one or more neoplastic malignant cells, the radiation therapy is administered before, during, and/or after applying the pulsed magnetic field to the one or more neoplastic malignant cells.

In some aspects of the methods of inducing apoptosis of one or more neoplastic malignant cells, the one or more neoplastic malignant cells do not comprise a malignant glycocalyx A.

In some aspects of the methods of inducing apoptosis of one or more neoplastic malignant cells, the neoplastic malignant cell cancer is a carcinoma, a sarcoma, a myeloma, a leukemia, or a lymphoma. In some aspects of the methods of inducing apoptosis of one or more neoplastic malignant cells, the neoplastic malignant cell cancer is skin cancer, breast cancer, prostate cancer, bladder cancer, cervical cancer, endometrial cancer, lung cancer, colon cancer, or rectal cancer. In some aspects of the methods of inducing apoptosis of one or more neoplastic malignant cells, the neoplastic malignant cell cancer is brain cancer. In some aspects of the methods of inducing apoptosis of one or more neoplastic malignant cells, the neoplastic malignant cell cancer is lung cancer.

In some aspects of the methods of inducing apoptosis of one or more neoplastic malignant cells, the pulsed magnetic field is administered for about 1 minute to about 60 minutes per session. In some aspects of the methods of inducing apoptosis of one or more neoplastic malignant cells, the pulsed magnetic field is administered for about 1, 5, 10, 15, 20, 25, 30, 35, 40, 45, 50, 55, or 60 minutes.

### 5. Methods of Increasing a Subject's Responsiveness to an Immunotherapy

Disclosed are methods of increasing a subject's responsiveness to an immunotherapy, comprising: a) obtaining or having obtained a sample from the subject, wherein the sample comprises at least one neoplastic malignant cancer cell; b) determining that the isolated neoplastic malignant cancer cell comprises a malignant glycocalyx B; and c) applying a pulsed magnetic field (PMF) to the subject, thereby increasing a subject's responsiveness to an immunotherapy.

In some aspects of the methods of increasing a subject's responsiveness to an immunotherapy, the method further comprises administering a therapeutically effective amount an immunotherapy to the subject. In some aspects of the methods of increasing a subject's responsiveness to an immunotherapy, the therapeutically effective amount the immunotherapy is administered before, during, and/or after applying the pulsed magnetic field to the subject.

In some aspects of the methods of increasing a subject's responsiveness to an immunotherapy, the method further comprises administering a therapeutically effective amount of chemotherapy to the subject. In some aspects of the methods of increasing a subject's responsiveness to an immunotherapy, the therapeutically effective amount the chemotherapy is administered before, during, and/or after applying the pulsed magnetic field to the subject.

In some aspects, the immunotherapy can be a checkpoint inhibitor. In some aspects, the terms “checkpoint inhibitors” and “immune checkpoint inhibitors” can be used interchangeably.

In some aspects, the methods of increasing a subject's responsiveness to an immunotherapy can further comprise administering one or more immunotherapies to a subject.

The methods disclosed herein comprise administering one or more checkpoint inhibitors to a subject. In some aspects, the checkpoint inhibitor can block CTLA-4 (cytotoxic T lymphocyte associated protein 4), PD-1 (programmed cell death protein 1) or PD-L1 (programmed cell death ligand 1).

In some aspects, the checkpoint inhibitor can be, but is not limited to, ipilimumab (Yervoy), pembrolizumab (Keytruda), nivolumab (Opdivo), cemiplimab (trade name Libtayo), and dostarlimab (Jemperli), atezolizumab (Tecentriq), durvalumab (Imfinzi), or avelumab (Bavencio), or a combination thereof. In some aspects, the checkpoint inhibitor can be, but is not limited to, Tremelimumab, Sintilimab (formerly IBI308; Tyvyt), Tislelizumab (formerly BGB-A317), Toripalimab (formerly JS 001), Spartalizumab (formerly PRD001); Camrelizumab (formerly SHR1210), KN035, Cosibelimab (formerly CK-301), CA-170, or BMS-986189, or a combination thereof.

In such embodiments the checkpoint inhibitor can be an inhibitor that blocks CTLA-4 (cytotoxic T lymphocyte associated protein 4), PD-1 (programmed cell death protein 1) or PD-L1 (programmed cell death ligand 1). In some aspects, the checkpoint inhibitor previously administered to the subject can be, but is not limited to, ipilimumab (Yervoy), pembrolizumab (Keytruda), nivolumab (Opdivo), cemiplimab (trade name Libtayo), and dostarlimab (Jemperli), atezolizumab (Tecentriq), durvalumab (Imfinzi), or avelumab (Bavencio), or a combination thereof. In some aspects, the checkpoint inhibitor can be, but is not limited to, tremelimumab, sintilimab (formerly IBI308; tyvyt), tislelizumab (formerly BGB-A317), toripalimab (formerly JS 001), spartalizumab (formerly PRD001); camrelizumab (formerly SHR1210), KN035, cosibelimab (formerly CK-301), CA-170, or BMS-986189, or a combination thereof.

In some aspects, the checkpoint inhibitor is a PD-1 inhibitor, PD-L1 inhibitor, or CTLA4 inhibitor. In some aspects, a PD-1 inhibitor can be, but is not limited to, Nivolumab, Pembrolizumab, or Cemiplimab. In some aspects, a PD-L1 inhibitor can be, but is not limited to, Atezolizumab, Avelumab, or Durvalumab. In some aspects, a CTLA-4 inhibitor can be, but is not limited to, ipilimumab or tremelimumab.

In some aspects, the methods of treating a neoplastic malignant cell cancer or methods of treating a malignant neoplasm can further comprise administering one or more chemotherapies to a subject. In some aspects, the chemotherapy can be an alkylating agent, plant alkaloid, antimetabolite, anthracycline, topoisomerase inhibitor or corticosteroid.

In some aspects, the methods of increasing a subject's responsiveness to an immunotherapy can further comprise administering a therapeutically effective amount of chemotherapy to the subject. In some aspects of the methods of treating a neoplastic malignant cell cancer, the therapeutically effective amount the chemotherapy is administered before, during, and/or after applying the pulsed magnetic field to the subject.

In some aspects, a chemotherapy can be, but is not limited to, an alkylating agent, an antimetabolite agent, an antineoplastic antibiotic agent, a mitotic inhibitor agent.

In some aspects, the chemotherapy can be, but is not limited to, docetaxel, carboplatin, cisplatin, oxaliplatin, Dacarbazine, procarbazine, temozolomide, Busulfan, Lomustine, bendamustine, cyclophosphamide, ifosfamide, Fluorouracil, Gemcitabine, Doxorubicin, Methotrexate, Capecitabine, Altretamine, Epirubicin, Irinotecan, Daunorubicin, Paclitaxel, Chlorambucil, Idarubicin, Cabazitaxel, Floxuridine, Pembrolizumab, Nivolumab, durvalumab, Cemiplimab, Ipilimumab, atezolizumab, Niraparib, Olaparib, Bevacizumab, alpelisib, Nab-paclitaxel (Abraxane), Sacituzumab govitecan, Dato-Dxd, taxol, Lenvatinib, everolimus, Letrozole, Gefitinib, Erlotinib, or sorafenib.

In some aspects, the chemotherapy can be one or more of Altretamine, Bendamustine, Busulfan, Carboplatin, Chlorambucil, Cisplatin, Cyclophosphamide, Dacarbazine, Ifosfamide, Mechlorethamine, Melphalan, Oxaliplatin, Procarbazine, Temozolomide, Thiotepa, Trabectedin, Carmustine, Lomustine, Streptozocin, 5-fluorouracil, 6-mercaptopurine, Azacitidine, Capecitabine, Cladribine, Clofarabine, Cytarabine, Decitabine, Floxuridine, Fludarabine, Gemcitabine, Hydroxyurea, Methotrexate, Nelarabine, Pemetrexed, Pentostatin, Pralatrexate, Thioguanine, Trifluridine/tipiracil combination, Etoposide, Irinotecan, Irinotecan liposomal, Mitoxantrone, Teniposide, Topotecan, Cabazitaxel, Docetaxel, Nab-paclitaxel, Paclitaxel, Vinblastine, Vincristine, Vincristine liposomal, Vinorelbine, Daunorubicin, Doxorubicin, Doxorubicin liposomal, Epirubicin, Idarubicin, Mitoxantrone, Valrubicin, Bleomycin, Dactinomycin, Mitomycin-C, all-trans-retinoic acid, Arsenic trioxide, Asparaginase, Eribulin, Ixabepilone, Mitotane, Omacetaxine, Pegaspargase, Procarbazine, Romidepsin, Vorinostat, Dexamethasone, Hydrocortisone, Methylprednisolone, Prednisolone, or Prednisone.

In some aspects of the methods of increasing a subject's responsiveness to an immunotherapy, the PMF is administered daily for about 7 days. In some aspects of the methods of increasing a subject's responsiveness to an immunotherapy, the PMF is administered daily for about 7 days, about 10 days, about 12 days, about 14 days, or about 21 days. In some aspects of the methods of increasing a subject's responsiveness to an immunotherapy, the PMF is administered daily for about 7 days during each round of immunotherapy. In some aspects of the methods of increasing a subject's responsiveness to an immunotherapy, the PMF is administered for about 7 days, about 10 days, about 12 days, about 14 days, or about 21 days during each round of immunotherapy.

In some aspects of the methods of increasing a subject's responsiveness to an immunotherapy, the PMF is administered at an amplitude of about 5-30 mT. In some aspects of the methods of increasing a subject's responsiveness to an immunotherapy, the PMF is administered at an amplitude of about 10-20 mT or about 15-20 mT, or at an amplitude of about 5 mT, 10 mT, 15 mT, 20 mT, 25 mT or 30 mT. In some aspects of the methods of increasing a subject's responsiveness to an immunotherapy, the PMF is administered at a 20 mT maximum field (dB/dt˜2 T/sec).

In some aspects of the methods of increasing a subject's responsiveness to an immunotherapy, the PMF is administered at a frequency of about 50 Hz to 385 Hz. In some aspects of the methods of increasing a subject's responsiveness to an immunotherapy, the PMF is administered at a frequency of about 100 Hz to 385 Hz, 150 Hz to 350 Hz, 200 Hz to 300 Hz, or at the frequency of about 50 Hz, 100 Hz, 150 Hz, 200 Hz, 250 Hz, 300 Hz, 350 Hz, or 385 Hz.

In some aspects of the methods of increasing a subject's responsiveness to an immunotherapy, the PMF is 20 mT maximum field (dB/dt˜2 T/sec) over a 10 msec duty cycle at 50 Hz.

In some aspects of the methods of increasing a subject's responsiveness to an immunotherapy, the PMF is administered at a duty-cycle pulse width of 70 μsec using 15 Hz pulse trains.

In some aspects of the methods of increasing a subject's responsiveness to an immunotherapy, the PMF is administered at a pulse duty-cycle rise time of about 5-15 msec. In some aspects of the methods of increasing a subject's responsiveness to an immunotherapy, the PMF is administered at a pulse duty-cycle rise time of about 5, 6, 7, 8, 9, 10, 11, 12, 13, 14, or 15 msec.

In some aspects of the methods of increasing a subject's responsiveness to an immunotherapy, the method further comprises administering radiation therapy to the subject. In some aspects of the methods of increasing a subject's responsiveness to an immunotherapy, the radiation therapy is administered before, during, and/or after applying the alternating electric field.

In some aspects of the methods of increasing a subject's responsiveness to an immunotherapy, the isolated neoplastic malignant cancer cell does not comprise a malignant glycocalyx A.

In some aspects of the methods of increasing a subject's responsiveness to an immunotherapy, the neoplastic malignant cell cancer is a carcinoma, a sarcoma, a myeloma, a leukemia, or a lymphoma. In some aspects of the methods of increasing a subject's responsiveness to an immunotherapy, the neoplastic malignant cell cancer is skin cancer, breast cancer, prostate cancer, bladder cancer, cervical cancer, endometrial cancer, lung cancer, colon cancer, or rectal cancer. In some aspects of the methods of increasing a subject's responsiveness to an immunotherapy, the neoplastic malignant cell cancer is brain cancer. In some aspects of the methods of increasing a subject's responsiveness to an immunotherapy, the neoplastic malignant cell cancer is lung cancer.

In some aspects of the methods of increasing a subject's responsiveness to an immunotherapy, the pulsed magnetic field is administered for about 1 minute to about 60 minutes per session. In some aspects of the methods of increasing a subject's responsiveness to an immunotherapy, the pulsed magnetic field is administered for about 1, 5, 10, 15, 20, 25, 30, 35, 40, 45, 50, 55, or 60 minutes.

### C. EXAMPLES

### Example 1

Mechanisms by which electric (E-) or magnetic (B-) fields might be harnessed to affect tumor cell behavior remain poorly defined, presenting a barrier to translation. Early studies show that the glycocalyx of lung cancer cells might play a role in mediating plasma membrane leak by low frequency pulsed magnetic fields (Lf-PMF) generated on a low-energy solenoid platform. In testing glioblastoma and neuroblastoma cells known to overexpress glycoproteins rich in modifications by the anionic glycan sialic acid (Sia), exposure of brain tumor cells on the same platform to a pulse train that included a 5 min 50 Hz Lf-PMF (dB/dt˜2 T/sec at 10 msec pulse widths) induced a very modest but significant protease leak above that of control non-exposed cells (with modest but significant reductions in long-term tumor cell viability following the 5-min exposure). Using a markedly higher dB/dt system (80 T/sec pulses, 70 μsec pulse-width at 5.9 cm from a MagVenture coil-source) induced markedly greater leak by the same cells, and eliminating Sia by treating cells with AUS sialidase immediately pre-exposure abrogated the effect entirely in SH-SY5Y neuroblastoma cells, and partially in T98G glioblastoma cells. The system demonstrated significant leak (including inward leak of propidium iodide), with reduced leak at lower dB/dt in a variety of tumor cells. The ability to abrogate Lf-PMF protease leak by pre-treatment with sialidase in SH-SY5Y brain tumor cells or with heparin lyase in A549 lung tumor cells indicated the importance of heavy Sia or heparan sulfate glycosaminoglycan glycocalyx modifications as dominant glycan species mediating Lf-PMF membrane leak in respective tumor cells. This “first-physical” Lf-PMF tumor glycocalyx event, with downstream cell stress, may represent a “tunable” transduction mechanism that depends on characteristic anionic glycans overexpressed by distinct malignant tumors.

Malignant tumors characteristically overexpress glycans that carry anionic charge at physiologic pH in the tumor-cell glycocalyx, a layer or “canopy” of complex carbohydrates that coats the cell-surface with protein core attachments to the tumor plasma membrane. Distinct from that of normal cells, the unique glycan-dense tumor glycocalyx may be susceptible to electromotive forces generated by Lf-PMFs over continuous tumor-cell surfaces, with tumor plasma membrane leak (and downstream cell stress) as a result of membrane shear forces or molecular torque imposed by EMF-pulsed movement of the overlying glycocalyx. This mechanistic understanding represents an opportunity for selective targeting of tumor cells in novel clinical platforms.

The use of oscillating magnetic fields to alter cancer cell growth has been described in biophysics, but the field strengths, frequencies, and/or characteristics to consistently induce cancer cell death or tumor regression in biological models remain a mystery (Huang et al 2022; Kielbik et al 2021; Tatarov et al 2011; Tofani et al 2001). This is more generally appreciated in light of a variety of biological effects that may result from very low to high intensities and/or frequencies of experimental and environmental magnetic fields (summarized in (Zadeh-Haghighi et al 2022). It was recently discovered that “pulsed” magnetic fields induce electromotive forces (EMFs) that engage with charged complex carbohydrate (glycan) molecules that occupy the “glycocalyx” of cancer-cell membranes (Ashdown et al 2020). This results in membrane leak and imposes a modest inhibition in tumor-cell viability in preliminary studies. Unlike cancer radiotherapy that targets high-energy ionizing radiation to S-phase DNA of rapidly dividing tumor cells, Low frequency narrow pulse-width (i.e., “sharp”) magnetic fields, characterized by a high dB/dt pulse quality, can be used to selectively couple with this unique cancer cell surface glycan layer (Ashdown et al 2020; Gospodinov et al 2013; Hart et al 2010). Under unique spatial and temporal conditions, this may disrupt tumor membrane integrity. Low-frequency oscillating magnetic fields have been shown to inhibit tumor growth in mice (Tatarov et al 2011; Tofani et al 2001; Nie et al 2013), although mechanisms, including unique effects of the pulsed-field magnitude and width (or pulse “sharpness” defined by dB/dt) on tumor membrane leak as well as any transducing capability of glycocalyx in this process have not been described. Thus, while efficacy in altering neoplastic cell growth in in vitro and in vivo models has been shown (Tatarov et al 2011; Tofani et al 2001; Koh et al 2008; Omote et al 1990), knowledge on biophysical mechanism(s), cancer-cell specific effects, and the connection between cellular physiology and in vivo applications remains undefined. Nevertheless, the majority of studies show an inhibitory effect on tumor cell growth. In some cases, very low frequencies are used (Tatarov et al 2011; Koh et al 2008; de Seze et al 2000; Zhang et al 2002). Future studies should consider how the tumor cell surface may transduce a “first physical effect,” such as membrane leak, with downstream cell stress that may follow.

Other studies have examined how magnetic pulsing conditions on distinct sets of neoplastic cell monolayers (and distinct glycocalyx compositions) may be susceptible to membrane-altering effects of low frequency pulsed magnetic fields (Lf-PMFs) with well controlled pulse intensities. Conceptually, a heavily charged glycocalyx expressed over a continuous surface layer on multiple adjacent tumor cells may ultimately be susceptible to a conductive electromotive force (EMF) induced by a pulsed magnetic field perpendicular to the cell-plane with magnitude proportional to dB/dt by Faraday's law of induction (Pall et al 2022), and where the magnitude of the EMF may be augmented if it bounds a larger area (A) of continuous glycocalyx over which dB/dt is fluxed [where EMF ˜A(dB/dt)]. This may alter membrane integrity with an effect sufficient to leak proteases, as measured by commercial cytotoxicity assays under defined Lf-PMF conditions in cultured tumor cells (Ashdown et al 2020). Neoplasms may overexpress distinct families of charged glycans typically found on the tumor-cell surface, including glycoprotein termini heavily modified by sialic acid (Sia) in addition to anionic glycosaminoglycans (i.e., sulfated species such as heparan sulfate and chondroitin sulfate) and hyaluronan (HA). With a specific focus on Sia, that monosaccharide contains an anionic negatively charged oxygen atom (at physiologic pH), and decorates the termini of poly antennary O-linked and N-linked glycans over-expressed on tumor glycoproteins (Kang et al 2018; Fuster et al 2005; Nagaranjan et al 2018; Pinho et al 2015; Rodrigues et al 2018; Seidenfaden et al 2003). While certain tumors overexpress distinct proteoglycans into the tumor glycocalyx, others over-express heavy Sia modifications on glycoproteins or polysialic acid (polySia) chains extended on glycan termini decorating unique tumor core proteins (Pinho et al 2015; Wade et al 2013; Chin-Hu et al 2018; Valentiner et al 2011).

A unique class of neoplastic cells that often overexpress Sia in the glycocalyx includes that of brain neoplasms such as glioblastoma and neuroblastoma (Pinho et al 2015; Falconer et al 2012). The latter often express polySia (e.g., as in the model SH-SY5Y cell line), which may project high levels of negative charge into the glycocalyx (Valentiner et al 2011), a property contributing to cell-cell repulsion which facilitates tumor tissue invasion and metastatic progression (Fuster et al 2005; Falconer et al 2012). Demonstrating how Lf-PMF induced membrane disruption may be sensitive to destroying or removing a specific anionic glycan in the glycocalyx of specific tumor cells may be helpful in defining the importance of that specific glycan in mediating the EMF-driven leak in that unique tumor type. Studies focused on Sia overexpressing brain tumor lines as models to study susceptibility and mechanistic dependence of tumor cellular protease leak patterns in response to controlled Lf-PMFs. This included consideration as to whether sialidase mediated clearance of Sia on tumor cells might render tumor cells less sensitive to Lf-PMF alterations in membrane integrity. A lung cancer cell line that is also susceptible to high dB/dt Lf-PMFs, was also examined albeit with unique sensitivity to the presence/absence of distinct charged glycans (heparan sulfate) that may at least functionally “dominate” the glycocalyx of such cells. It should be noted that brain tumors (especially high-grade gliomas and neuroblastomas) often overexpress sialic acid heavily on the glycocalyx (Pinho et al 2015; Falconer et al 2012; Amoureux et al 2010; Wielgt et al 2021), which correlates with their aggressiveness. This includes well-documented overexpression in glioblastoma lines T98G, A172 (Cuello et al 2020; Bartik et al 2008) and the neuroblastoma line SH-SY5Y (Valentiner et al 2011). So as a proof-of-concept herein, magneto-sensitivity of brain tumors was evaluated, and Sia as a brain-malignancy anionic glycan overexpressed on the brain tumor glycocalyx.

Findings suggest the importance of EMF-mediated forces by Lf-PMFs on tumor-cell glycocalyces composed of a considerable presence of Sia in representative brain tumor cell lines, and where transduction of such forces via attachment to membrane-bound core proteins may induce membrane stress and leak. On the other hand, distinct glycans appear to mediate the effects in distinct tumors; but to test principles, a relatively high-intensity dB/dt could practically be applied using a transcranial magnetic stimulation (TMS) type system to drive uniquely configured coils to accommodate cultured cells in perpendicularly fluxing fields across the cells. To date, this system has not been applied to tumor cell systems, and the role of the malignant-cell glycocalyx has not been examined as a mechanical transducer of dB/dt driven EMFs in distinct tumor types with distinct anionic glycan compositions. The insights may facilitate rational translational considerations from the mechanistic principles suggested herein.

### Methods

Cell culture and field-exposure preparations: Human glioblastoma cell lines T98G and A172 were grown in cell culture at 37° C. in EMEM and DMEM minimal essential culture media, respectively, supplemented with 10% fetal bovine serum (FBS); and the human neuroblastoma cell line SH-SY5Y was grown in EMEM containing 10% FBS. The human lung adenocarcinoma cell line A549 was grown in F-12K medium supplemented with 10% FBS. Cells were also supplemented with 1% PenStrep (GibCo), and subcultured once they reached 80% confluency using a 0.5% Trypsin 0.2% EDTA solution to lift cells; and plated into either 96-well or 12-well format, depending on the assay being used. For most studies involving magnetic field exposures, cells were seeded at 1.0×10⁴ cells per well in 96-well (96W) cell culture plates, and allowed to grow for 48 hr prior to magnetic field exposure (when cells were typically approximately 70-90% confluent). As a typical primary cell type (immune monocyte) that might be found in the tumor microenvironment, cultured primary bone marrow derived dendritic cells (BMDCs) were isolated as in (Gupta et al 2020), and seeded into 96W plates at day 7 of primary-cell growth at 5.0×10⁴ cells per well, and settled over 3 days prior to magnetic field exposure. For a subset of studies (employing a low-intensity solenoid pulsed magnet field platform), T98G tumor cells were seeded at 2.0×10⁴/well in 96W plates, while settling overnight prior to magnetic field exposure, with a similar 70-90% confluence achieved prior to field exposure. For cultured cell-growth studies following field exposure, cells seeded at 1.0×10⁵ in 12W plates were exposed to low-intensity magnetic fields on a solenoid platform followed by daily cell counts for 3 days growth post-exposure.

Magnetic field platforms and exposures: For most experiments, a low frequency pulsed magnetic field (Lf-PMF) system was used. The source coil was connected to a MagVenture unit used for Transcranial Magnetic Stimulation (Drakaki et al 2022), with application using a Cool 40 Rat-Coil (Parthoes et al 2016) trough-shaped platform generating dB/dt in a 2-D ring-shaped spherical-field cross section from the trough base (coil center). The trough was laterally oriented with a 96-W plate inserted in the field so that fluxing B-lines would run perpendicular to (and across) the plated monolayer tumor cells on 96W plate-bottom surfaces. Magnetic pulses were delivered to plated cells at a relatively high ratio of amplitude to pulse-width for a 5 min exposure period at room temperature, and consisted of oscillating fields with a duty-cycle pulse width of 70 μsec using 15 Hz pulse trains, with dB/dt˜80 T/sec for 96W plated cells centered and fluxed over a high-intensity zone positioned 5.9 cm from the magnetic coil-center (“near” zone) versus ˜1.8 T/sec at a more distal zone (“far” zone) of exposure positioned 10.5 cm from the coil center. These values correspond to the running unit output set at 10% maximum (relative to maximum dB/dt by the coil source) for all 5-minute runs to ensure constant output conditions with optimal standardized coil cooling for the above pulse-train settings. In a subset of experiments, to reference an original prototype platform, some plated tumor monolayers were exposed to a simple low-intensity oscillating B-field solenoid platform (used as in (Ashdown et al 2020)) placed immediately below the plate, emitting fields driven by a 10 V power source and a pulsing circuit to generate 20 mT maximum-amplitude oscillating fields over two sequential 5 min trains at 50 Hz and 385 Hz, respectively (10 min total exposure), with a pulse duty-cycle rise time of approximately 10 msec. This low energy Lf-PMF pulse-train exposure was used for initial exposures of T98G and SHSY brain tumor cell lines with reference to original studies using A549 lung cancer cells. For all experiments, control cells plated under equivalent conditions were incubated in parallel time course to that of Lf-PMF exposed cells, without magnet exposure under otherwise identical conditions.

Plasma membrane integrity assays: Cellular membrane integrity to outward protease leak was assayed immediately after Lf-PMF exposures using a luciferase based cytotoxicity assay (CytoTox Glo G9291 Promega) according to manufacturer instructions for 96W plate applications. The assay measures protease release into the medium of plated cell monolayers, with cells typically seeded in 96W plates 48 hr prior to Lf-PMF exposure (with additional cell media and handling conditions as detailed in “Cell culture” section above) to allow for plate-attachment and establishment of robust sub-confluent cell monolayers. To examine whether the effects of a robust Lf-PMF exposure on leak is reversible, the field effect (i.e., protease release) was checked after a 15 min recovery time in culture, with replacement of fresh medium immediately following the 5 min Lf-PMF exposure period, and with comparison to sham-treated cells (i.e., transport of plate to magnetic platform area, but no magnetic exposure) treated otherwise under identical conditions. In this way, continued protease release (i.e., irreversible/continuous cell stress) following a short recovery period was assessed.

As an additional proof-of-concept test of the induction of inward leak by Lf-PMFs, model 549 cells were grown on 96-well cell culture plates to near confluency in 100 mL growth medium. Propidium Iodide (PI) was added to appropriate wells at 25 mM, and cells were exposed to low frequency magnetic pulsing conditions as previously published (Ashdown et al 2020) over a low-energy solenoid platform. PI supplemented media was removed after magnetic exposure and the wells were washed twice with PBS. The wells were then filled with 100 mL of PBS added to each well before assaying PI signal on a fluorescent plate reader.

Sialidase enzymatic digestion and validation studies: For studies in which sialic acid (Sia) digestion from cell-surface glycans was employed, cells were exposed to 5 U/mL of AUS Sialidase (Roche) diluted in normal growth media for 1 hr at 37° C. (For magnetic field exposures post-sialidase treatment, plated cells were exposed to magnetic field immediately after sialidase incubation.) Sialidase activity was validated by examining the binding of the biotinylated lectins MAL-II (Vector B-1265) or SNA (Vector B-1305) to cultured, freshly harvested tumor cells +/−treatment with AUS sialidase. The lectins were incubated with the cells for 1 hr at 4° C. Streptavidin-PE (Biolegend 405203) was added to the lectin labeled cells with appropriate washings, and signal was assayed by flow cytometry.

Heparinase enzymatic digestion and validation studies: To destroy heparan sulfate (HS) glycosaminoglycan chains exhaustively on the cell surface, heparin lyase III (Hep'ase) was used for 1 hr at 12.5 mU/ml at 37° C. Cells were washed with phosphate buffered saline (PBS), followed by addition of fresh medium prior to subsequent experimental exposures. Testing of fluorescent FGF-2 binding to the cell surface was carried out by incubating biotinylated FGF-2 for 30 minutes followed by washing and streptavidin-phycoerythrin (PE) labeling, and flow cytometry: This was carried out to assess cell surface HS ligand binding capacity (as in (Fuster et al 2007)), where labeled FGF-2 is used as a well-established cell surface HS probe (Yayon et al 1991), and measured as a reporter for binding by flow cytometry on cells treated with and without Hep'ase, to assess HS digestion efficacy.

Cultured cell proliferation assays: Beginning at the time of magnet exposure (24 hr after cell-seeding at 1×10⁵ cells per well into 12w plates, cell growth was measured through daily cell counts harvested from plates over a 3 day time period. On any given day, 0.5% Trypsin 0.2% EDTA solution was used to harvest cells from plates, and cells were counted through use of an automated hemocytometer (Countess II, Life Technologies).

Reactive Oxygen Species (ROS) generation assays: A549 tumor cells were incubated with 10 mM H2DCFDA ROS-sensitive fluor-reagent (DCF; Invitrogen D399) in PBS for 30 min at 37° C., similar to reagents and methodology in (Kim et al 2020). All non-DCF cells were incubated in PBS for the same time period. Cells were returned to normal growth media after treatment and incubated for 30 min at 37° C. A baseline reading was performed after incubation. Some cells were exposed at room temp to the standard 5 min exposure to a MagVenture (centered at the high-intensity zone; 80 T/sec, 70 μs pulse width) magnetic field while others (controls) rested un-exposed on the same bench. Plates were then assayed for fluorescence at Time 0, 30 min, 1 hr, and 3 hr post-exposure. (In an additional subset of cells, heparanase pre-treatment prior to Lf-PMF exposure was used as an additional control to eliminate heparan sulfate from the cell surface.) Fluorimeter (FITC-detection) measurements for the DCF signal were then made on a 96-W plate-reader fluorimeter (Becton), and data was averaged from multiple (Ashdown et al 2020) replicate measures per condition. Fluorescence signal of cells in same media but otherwise unexposed to DCF at the same time points was used as the background signal for each time point.

For most experiments, means were compared using students T-test with p-value for significance at a cutoff of 0.05. Paired T-tests were used where appropriate (e.g., comparing paired data for mean protease leak following Lf-PMF exposure versus mean for Lf-PMF exposure post-enzyme treatment in paired-measurement same-platform experiments). For all experiments, mean (reported with+/−SD) for multiple wells treated under unique conditions (e.g., no-magnet control versus Lf-PMF exposure) was used for statistical comparison. If multiple experiments were carried out with high variability in mean control values, data normalized to 1.0 (for control) were tested for mean stimulation values significantly above 1.0 in multiple experiments, with significance (for mean+/−SD for n trials) reported using the one sample T-test. In the rare case where multiple Lf-PMF exposure measurements relative to control were recorded on a different day than multiple Lf-PMF measurements following Hep'ase treatment relative to control, then the appropriate unpaired T-test was used (comparing means in two independent and unpaired data-sets). Number of trials (n) for each set of experiments is indicated in figure legends, along with p-values.

### Results

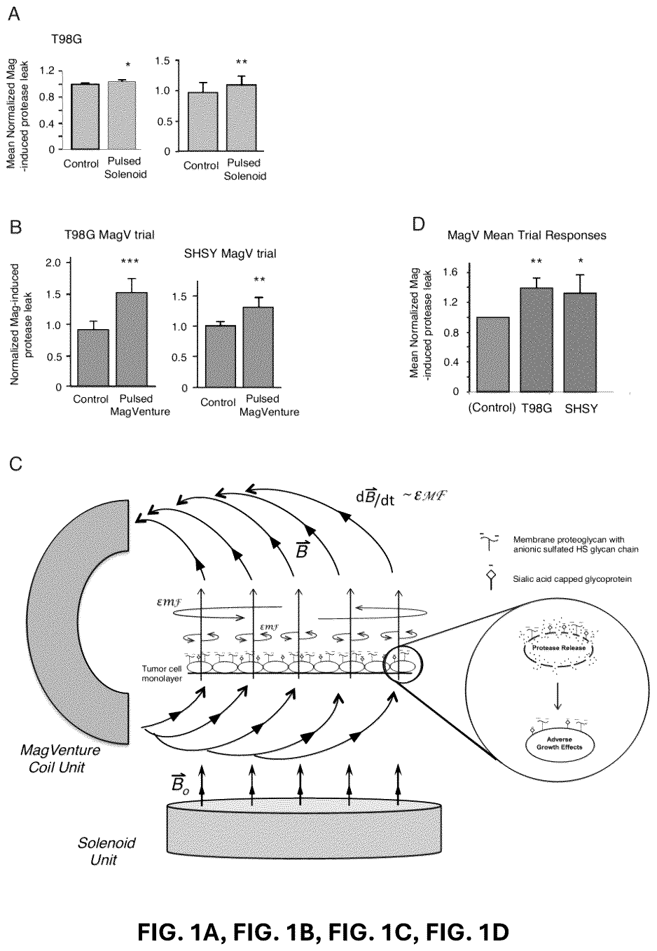

Exposure of brain tumor cells to distinct Lf-PMF platforms affects membrane protease leak. A variety malignant brain neoplasms over-express anionic-charged glycans, including Sia on a variety of glycan termini as well as anionic glycosaminoglycan polymers on membrane proteoglycans that may play roles in transducing EMFs driven by an external Lf-PMF source (Ashdown et al 2020; Pinho et al 2015; Chin-Hu et al 2018). To examine how such transduction may affect tumor membrane integrity, 96-well plated T98G glioblastoma and SH-SY5Y neuroblastoma monolayers (named “SHSY” herein) at sub-confluence were exposed to a low-intensity Lf-PMF solenoid platform delivering a 20 mT maximum field (dB/dt˜2 T/sec) with pulses over a 10 msec duty cycle at 50 Hz (as originally employed in (Ashdown et al 2020)). After a 5 min exposure at room temp, cells were immediately assayed for protease leak using a commercial luciferase-based assay. While a very modest but significant leak was detected immediately following Lf-PMF exposure compared to that of parallel control non-exposed cells (FIG. 1A), exposure over the same period to a high dB/dt system (MagVenture coil, driving Lf-PMF 80 T/sec pulse trains across plated cells over a 70 μsec duty cycle at 15 Hz) resulted in markedly increased leak in both T98G and SHSY cells, as demonstrated in individual representative experiments (FIG. 1B) as well as the means of multiple trials (FIG. 1D). In this high dB/dt system, plated cells were placed within a pulsing magnetic field running perpendicular through 96-well plated cell monolayers, and protease leak appeared to be accordingly greater than that for cells placed over the much lower dB/dt solenoid platform (compare to FIG. 1A).

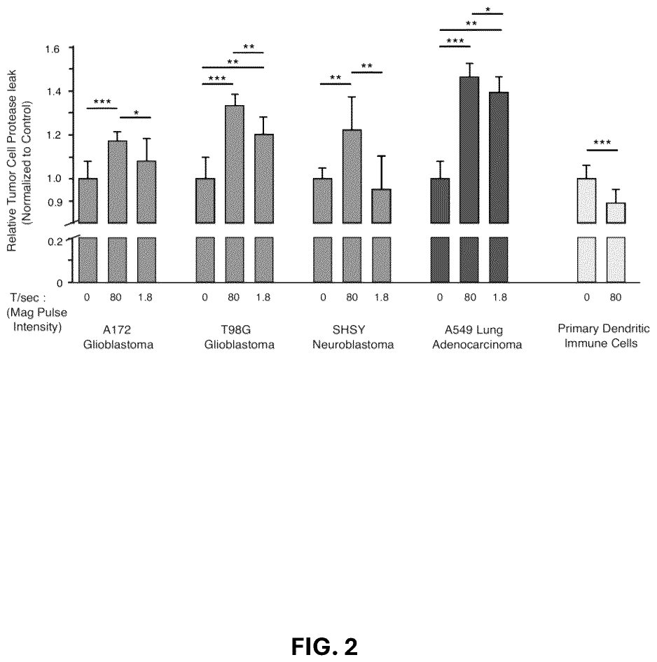

Common response patterns by a variety of malignant cells exposed to high performance Lf-PMFs. Using a high-intensity MagVenture coil (as in FIG. 1C), robust sensitivity of several tumor cell lines was demonstrated, including: 3 malignant brain tumor lines, A172 glioblastoma, T98G glioblastoma, SHSY neuroblastoma, and a model lung carcinoma cell line A549 (originally used in low-intensity solenoid platform, as in (Ashdown et al 2020)). Plated A172, T98G, SHSY, and A549 cells were exposed to two field intensities pulsing with magnetic flux perpendicular to the plated cells: (i) High dB/dt magnetic pulsing, with plated cells in a 96-well cluster immediately within the highest intensity zone of the MagVenture trough-shaped coil system (dB/dt˜80 T/sec) and (ii) lower dB/dt (˜1.8 T/sec) through cells clustered on a plate position a few cm distal to the highest intensity zone (not shown). Accordingly, a significant fall-off in Lf-PMF induced protease leak with distance from the trough center was observed; and with the fall-off in T/sec flux strengths with distance predicted from published (MagVenture, Cool-40 Rat Coil) (Parthoens et al 2016) field-strength maps from which dB/dt magnitudes could be estimated for 96-well cell clusters positioned near-(dB/dt intensity of 80 T/sec) and far-(dB/dt intensity of 1.8 T/sec) from the magnetic coil source (FIG. 2 tumor cell-line data). Exposure of primary mouse dendritic immune cells (as non-tumor host cells of interest in a tumor microenvironment) to Lf-PMF under the same conditions did not result in leak following exposure (FIG. 2, right). Thermal effects of magnetic pulsing could be a consideration in generating protease leak; however, significant differences in temperature was not observed on the surface of media-filled wells as a result of Lf-PMF pulsing, comparing near-coil well bases exposed to Lf-PMF˜80 T/sec to distal field exposed wells (˜1.8 T/sec), and to non-exposed (control) wells during real-time experimental bench-top conditions. It was also considered whether there was any immediate (ir) reversibility of such pulsed field effects on leak, by examining one of the model historical tumor (A549) cells under stringent conditions, employing Lf-PMF exposure at the high-intensity (80 T/sec) condition while checking for continued protease leak early (i.e., within 15 min) following a media wash at the completion of the 5 min Lf-PMF exposure. Under these conditions, increased protease leak by Lf-PMF exposed cells was not detected compared to basal protease leak by control (unexposed) cells over the same immediate-recovery period. Examining for irreversibility at lower intensities was thus moot; therefore, no unique effect of Lf-PMF exposure on protease leak during the early recovery period. This suggests that any immediate post-Lf-PMF membrane leak under these conditions was reversible. Downstream tumor-cell effects, however, may be another matter.

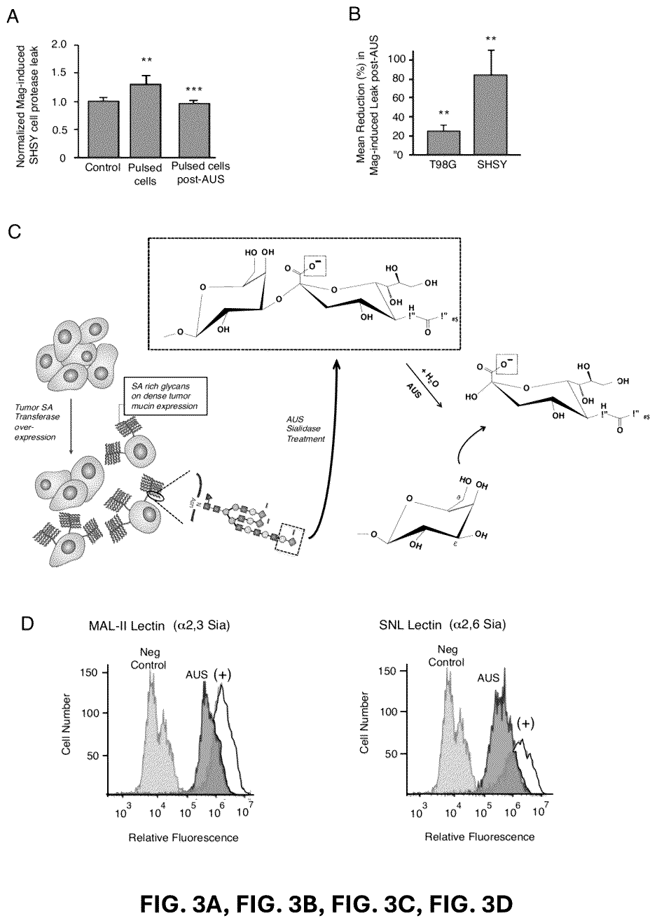

Impact of sialic acid elimination from the brain tumor glycocalyx on Lf-PMF leak. A variety of brain tumor cell lines uniquely over-express cell-surface Sia, including the T98G glioblastoma and SHSY neuroblastoma cell lines. SHSY neuroblastoma cell lines express polysialic acid (polySia). Less is known about cell-surface glycans and Sia over-expression on glioblastoma cells (or neuroblastoma cells) as a class. However, the literature (Pinho et al 2015; Chin-Hun et al 2018; Valentiner et al 2011) reports that Sia, which is anionic at physiologic pH, is frequently over-expressed in the glycocalyx of several tumors, including brain neoplasms. Thus, it was examined if Sia might play a role in transducing Lf-PMF induced EMFs as a electro-mechanical “coupling” component in the glycocalyx brain tumor cells. Strikingly, the marked Lf-PMF induced protease leak achieved by a short period of MagVenture pulses (i.e., 80 T/sec dB/dt pulsed at 15 Hz, as shown in FIG. 1B) could be abrogated by pre-treating the monolayer-plated SHSY cells with AUS sialidase, which removes cell-surface Sia, immediately prior to Lf-PMF exposure (FIG. 3A, right bar). More generally, while multiple trials with results similar to that of FIG. 3A demonstrated complete or near-complete abrogation of the high-intensity Lf-PMF mediated leak in SHSY cells by pre-treatment with AUS sialidase, trials using T98G cells demonstrated a lower (albeit still significant) effect of AUS pre-treatment in inhibiting the Lf-PMF driven leak. The data suggests that glycocalyx SA on T98G cells may play a lesser role in transducing pulsed EMF mediated leak on T98G cells than that of than that of SHSY cells (FIG. 3B). Note near-complete abrogation of leak in SHSY cells pre-treated with the enzyme (FIG. 3B, right bar). (As an additional control, baseline leak in AUS treated cells not exposed to Lf-PMF was not significantly different from non-AUS control baseline). AUS mediated digestion of Sia from the glycan termini of mucin glycoproteins over-expressed on tumor cells (FIG. 3C cartoon), resulting in the release of negatively charged Sia from the glycocalyx through AUS mediated Sia hydrolysis. The presence and marked reduction in surface Sia in AUS-treated SHSY cells was observed via flow cytometry using fluorescent lectins (MAL-II and SNL). MAL-II and SNL bind to surface glycans expressing either α2,3 linked (MAL-II specific) or α2,6 linked (SNL specific) terminal Sia (Zhang et al 2018) on the surface of SHSY cells. Treatment with AUS markedly reduced binding of both lectins to SHSY cells (FIG. 3D).

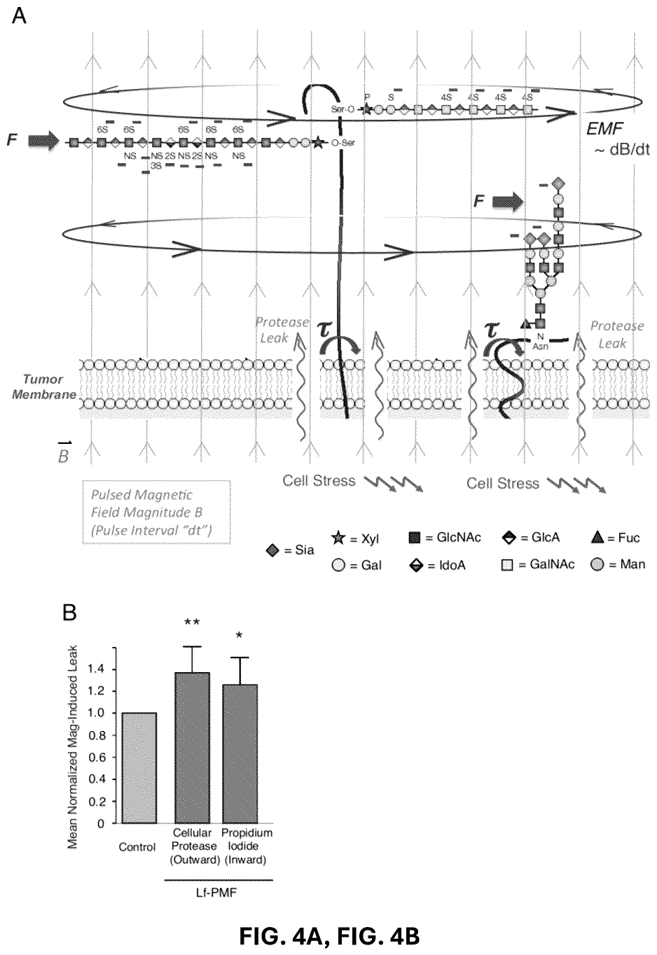

A biophysical basis for induction forces on the tumor glycocalyx during magnetic pulsing. As a general model of anionic glycan components in the tumor glycocalyx with which EMFs driven by Lf-PMFs may interact, distinct tumor cells overexpress certain glycoproteins and proteoglycans with heavily expressed anionic/charged glycan components projecting into the glycocalyx. In a variety of brain tumor cells, Sia overexpressed on glycoproteins may “dominate” as an anionic glycan species in the tumor glycocalyx. Alternatively, sulfated anionic glycosaminoglycan chains of heparan sulfate (HS) or chondroitin sulfate (CS) may be overexpressed by a variety of distinct tumors (e.g., HS on A549 lung carcinoma as an example), with anionic glycan chains that can transduce pulsed magnetic field induced EMFs into molecular/glycocalyx “motion” (FIG. 4A). target molecules for magnet-induced EMF motion and resultant tumor cell membrane disruption consist of charged glycans that are over-expressed in a glycocalyx “canopy” above the plasma membrane of carcinoma cells. The application of oscillating magnetic fields (illustrated by vertical B-field lines in FIG. 4A) with induced EMFs proportional to dB/dt (with pulse interval dt) allows for engagement of induced EMF/voltage paths orthogonal to the pulsed magnetic field lines with charged anionic tumor glycans in the glycocalyx. The induced electromotive force may impart torque (FIG. 4A) about the base/attachments of core proteins to the membrane, leading to membrane leak and disruption in membrane integrity and tumor cell stress. The downstream induction of reactive oxygen species (ROS) as well as parallel anti-oxidant pathway induction has been described following the exposure of mammalian cells to pulsed magnetic fields under a variety of conditions (Wang et al 2017). Control non-exposed model tumor cells appeared to demonstrate a steady mild increase in ROS generation over a 3 hr observation period of serial ROS measurements following sham-exposure (i.e., plated control cells on the bench away from the magnetic field at room temp for 5 min). In contrast, magnet-exposed cells subjected to a standard narrow pulse-width MagVenture Lf-PMF pulse train (5 min, 80 T/sec, 70 μs pulsing) showed an early significant increase in ROS production at 30 min post-exposure (i.e., significantly higher relative to baseline than that of control cells). Additionally, magnet exposed cells also illustrated a relative “dip” in ROS generation at the 1-hr post-exposure point (not shown), after which the cells showed a similar ultimate rise in signal relative to baseline controls at 3 hour post-stimulation. This “fluctuation” compared to controls was noted with time-point comparisons in serial ROS measurements. Eliminating heparan sulfate from the tumor cell-surface immediately prior to the exposure did not significantly change the temporal ROS profile following Lf-PMF exposure under these conditions (data not shown).

In order to also confirm Lf-PMF induced leak through an alternate method, inward leak of an exogenous standard indicator (propidium iodide; PI) was assessed during exposure of model A549 tumor cells to low frequency magnetic pulsing conditions. A well-established solenoid field was used to reference results to that of protease leak (as an outward Lf-PMF induced leak) under previously documented conditions using the same tumor cells (Ashdown et al 2020). Interestingly, inward leak of exogenous PI into tumor cells immediately after the short Lf-PMF exposure was significantly greater than that of control cells unexposed to the magnetic field, but otherwise under identical conditions (FIG. 4B). The degree of leak in Lf-PMF exposed tumor cells over that of control during the exposure period compared to the magnitude noted for outward leak of protease using a CytoTox assay system in the same cells under the same exposure conditions (comparing protease and PI bars in FIG. 4B).

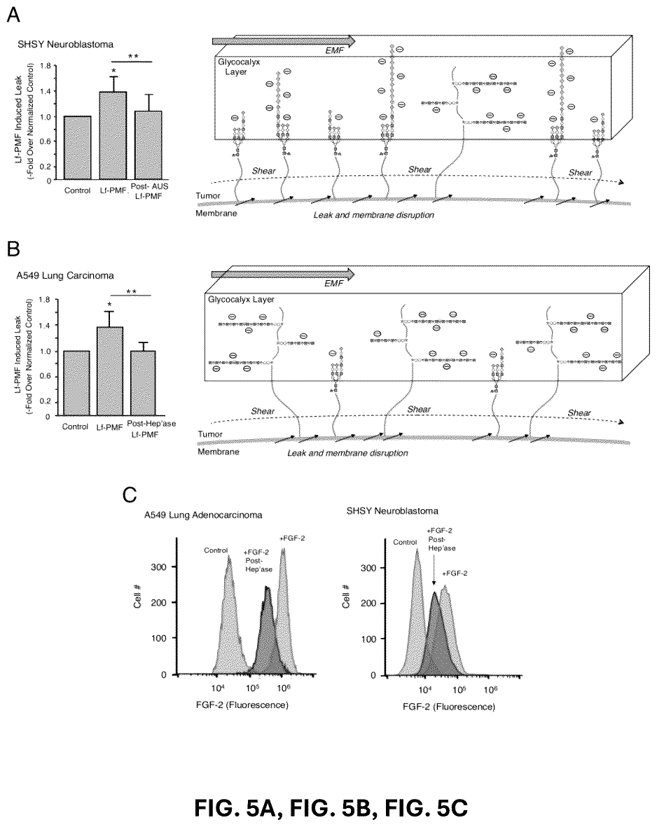

Distinct glycan species serve as major EMF-transducers in distinct tumor-glycocalyx systems. In a series of (n=4) independent trials paired with AUS testing wherein a mean dB/dt˜80 T/sec was employed in SHSY cells, and where the mean protease leak is nearly 40% over background in a 5 min period of Lf-PMF exposure (FIG. 5A graph, middle bar), exhaustive digestion of cultured cells with AUS was sufficient to achieve near-complete abrogation of cellular Lf-PMF induced leak following enzyme treatment (i.e., to under 10% over background; FIG. 5A graph, right bar). Conceptually, this is consistent with glycocalyx dominated by Sia modifications (FIG. 5A, illustration; right) as the major anionic species, and where susceptibility to Lf-PMF induced EMFs falls markedly after digesting Sia as a “dominant” glycan in the system that may be largely responsible for the ability of SHSY glycocalyx to mediate electro-mechanical transduction in this context. From another standpoint on the quantitative importance of Sia on magneto-sensitivity, while one could quest to experimentally boost Sia expression and thus magneto-sensitivity by feeding tumor cells increased Sia in the medium as a proof-of-concept, cells bearing high cell-surface Sia expression (e.g., tumor cells herein) appear to be resistant to such attempts as shown in metabolic labeling studies (Oetke et al 2001). Nevertheless, by another rationale comparing Sia surface expression in distinct tumor-specific cell lines, it is noteworthy to consider Sia expression in the glioblastoma cell lines A172 and T98G, which show distinct magneto-sensitivity when exposed to identical Lf-PMF pulsing conditions: In particular, Lf-PMF exposures (as shown in FIG. 2) resulted in greater impairment in membrane integrity in T98G cells as compared to A172 cells. Accordingly, using flow cytometry to examine MAL-II lectin binding (which binds to a predominant motif of terminal α2,3 linked Sia or alternatively to a non-Sia sulfated galactose motif (Bojar et al 2022)), a greater lectin affinity (>40% increase in mean fluorescence) was observed for T98G cells compared with A172 cells. Additionally, a markedly greater reduction in binding following AUS sialidase treatment of T98G cells (92% reduction) as compared to A172 cells (64% reduction) was also observed. This greater sialidase sensitivity suggests a markedly higher Sia presence on the T98G cell surface and a correlation with magneto-sensitivity.

On A549 lung adenocarcinoma cells, cell-surface HS appears to be a glycan that may be involved in transducing membrane leak in response to Lf-PMFs. Previous studies suggest that HS glycosaminoglycans may play a role as mediators of a Lf-PMF effect in this context, as the Lf PMF effect was completely abrogated in A549 cells pre-treated with exhaustive heparinase mediated clearance of cell-surface HS prior to the MagVenture 80 T/sec Lf-PMF stimulus (FIG. 5B graph, and cartoon to right). Interestingly, while HS glycosaminoglycans may serve as a “dominant” EMF-sensitive glycan in the A549 glycocalyx, additional studies showed that pre-treatment of A549 cells with AUS sialidase prior to the Lf-PMF exposure was sufficient to inhibit Lf-PMF induced protease leak by approximately 70%. This suggests a complementary effect of Sia in mediating Lf-PMF effects on the “HS dominant” tumor glycocalyx of A549 cells.

On the subject of HS as a dominant glycan in A549 glycocalyx, and the distinct compositions of glycocalyces in A549 versus SHSY cells, it is notable that upon testing for HS presence (and sensitivity to digestion) on the surface of these tumor cells, very unique patterns are found. A fluorescent FGF-2 probe was used to detect HS on the cell-surface of A549 and SHSY cells. While A549 cells are generally characterized by marked shifts in the mean fluorescence intensity for FGF-2 binding (as well as reduction upon heparinase digestion), SHSY cells showed markedly lower shifts (FIG. 5C), indicating likely greater levels of HS in the glycocalyx of A549 cells compared to that of SHSY cells (consistent with quantitative findings by others (Yue et al 2021)).

### DISCUSSION

Low-frequency oscillating magnetic fields have been applied to tumor cells in culture as well as in vivo tumor models, with striking inhibition of tumor growth in some platforms (see Tatarov et al 2011; Tofani et al 2001; Nie et al 2013; Omote et al 1990) as examples). However, mechanisms remain undefined and speculative for the most part. This is a barrier to translation and biological control. Unique properties of magnetically-induced or direct electric fields, with either capable of imparting EMF on charge (q) in the targeted space, include pulse frequency, pulse width (duty cycle), and maximum driving magnetic (B-field) magnitude. For electromagnetic waves employed directly as E-fields, frequencies capable of altering tumor membranes (demonstrated in the kHz range (40)) or as tumor treating fields (TTFs) (Rominiyi et al 2021) and directional amplitude (pointing vector) may be variables. However, electric fields are greatly attenuated at relatively short tissue depths. Pulsed B-fields were manipulated in bench-top cell based mechanistic studies, recalling the deep tissue-penetrating inductive power of pulsed magnetic fields. With either modality, variables that best “tune” a physical coupling with tumor-cellular structures (e.g., membrane, cytoskeleton, nuclear chromatin) remain a mystery. Moreover, “receiver” characteristics of the cellular or tissue elements with which such physical stimuli couple or interact have also not been well described. Even with some descriptions of the effects on cell behavior, how physical stimuli might be transduced remains a mystery. Although viability effects are not the focus herein, exposure of lung cancer cell monolayers to an oscillating solenoid platform generating Lf-PMFs over a 10 min period was shown to sufficiently induce a modest but significant inhibition of tumor-cell monolayer growth over subsequent days in culture relative to that of unexposed cells (Ashdown et al 2020). Moreover, the dB/dt intensity data herein (e.g., as in FIG. 2) supports that at least in the generally low (or ultra-low) frequency range, as long as the duty cycle for the pulse is far under the period of the pulse train, the dominating variable that affects membrane stress and leak may be the narrow pulse width rather than the absolute frequency per se. The data presented herein shows that augmented membrane leak is in response to increased dB/dt intensity, which determines the amplitude of the induced EMF. If a relatively “sharp” pulse can take place within the pulsing period, the ultimate frequency may not be as relevant as a short rise-time for the pulsed field, allowing for potentially substantial biological effects that may be “tunable” by adjusting pulse width or B-field amplitude (dB/dt determinants) at very low frequencies.

In order to understand how Lf-PMF induced EMFs might be transduced via unique properties of glycans in the glycocalyx of brain malignancies, brain tumor cells that commonly overexpress charged complex carbohydrate elements that may theoretically transduce the EMFs were tested. Tumor plasma membrane leak (as originally measured in (Ashdown et al 2020)) might ensue as a result of molecular attachment of the rich anionic EMF-susceptible tumor glycocalyx to the tumor membrane, with shear forces at points of membrane attachment (Hart 2010; Fuster et al 2005; Pinho et al 2015). Leakage of cellular proteases induced by Lf-PMF exposures of T98G glioblastoma and SHSY neuroblastoma cells (known to overexpress glycans richly modified by anionic Sia residues) was examined, and with a theoretically distinct composition to that of Lf-PMF susceptible A549 lung carcinoma glycocalyces that appear to be heavily endowed with anionic sulfated glycosaminoglycan chains. The basic observations of Lf-PMF induced leak among all 3 lines of cells with their unique responses in distinct platforms and sensitivity to glycan modifications herein may provide mechanistic insights regarding how tumor glycocalyx might electromechanically transduce EMFs to altered membrane integrity and leak for which downstream effects on tumor cell behavior and survival may follow. In that light, low-intensity solenoid exposure of both T98G and SHSY tumor cells to a short Lf-PMF period was sufficient to induce a low but significant level of membrane leak (FIG. 1A), the same short exposure was sufficient to modestly inhibit tumor monolayer growth and viability when cultured cells were quantified for 3 subsequent days in culture following short 5-min Lf-PMF exposures. The tumor viability effects are consistent with prior findings, and intriguing as effects secondary to transduction event(s) examined herein.

Induced EMFs by pulsed magnetic fields generated unique leak effects caused by thermal background energy (which could also lead to a basal amount of protease leak). The data presented herein demonstrated a “dose” effect within an expected range from the coil source using the MagVenture unit. Thus, as seen in distinct tumor cell lines in FIG. 2, a significant drop-off in Lf-PMF induced leak occurs at a distance from the coil over which the B-field magnitude (and thus dB/dt) decreases markedly, albeit still with significantly greater leak than control at that lower-magnitude distance from the coil. This implies that pulsed fields at both positions must induce effects over that of thermal background (control condition); however, estimating the exact amount of energy above the thermal background is difficult since glycocalyx forces and shear effects over multiple cells may likely be operative. Indeed, using a very narrow pulse width (i.e., “dt” in dB/dt with values under 100 μs) may be favorable, along with the ability to simultaneously recruit charge in the entire glycocalyx “slab” over multiple cells. The wider spatial area (i.e., multi-cellular contiguous glycocalyx) over which the bounding EMF may interact with the glycocalyx “slab” of charge and mass, along with the use of narrower pulse widths (greater dB/dt) employed in any Lf-PMF platform, may induce substantial shear stress on tumor plasma membranes (as illustrated in FIG. 4A and FIG. 5A-5B). In macroscopic tumor systems, the dB/dt “driving” effects for EMFs across macroscopic tumor fields might thus be “tunable” to generate greater cell-stress with adjustments in pulse width (thus dB/dt) without necessarily changing pulse frequency or B-field magnitude, although one should not expect to necessarily observe “linear” downstream biological effects.

The data presented herein demonstrates dose effects and sensitivity of the induced membrane leak to glycan elimination, implicating energy phenomena operating well above the thermal background experienced by resting sham (non-pulsed) control cells. While tempting to somehow estimate this using B-field driving variables in the system, it is very difficult to predict actual electromotive energies (in Joules) that impact the glycocalyx of single cells versus multiple cells as monolayers in the system. Moreover, the unique energies achieved using B-fields at narrow (milli- or micro-second) pulse widths must be taken into account. Narrowing the pulse width may impart additional order(s) of magnitude to the induced EMFs and energies, according to Faraday's law under a classical model (EMF dB/dt). Indeed, the narrow pulse width may help achieve “driving” energies at orders of magnitude above the thermal background, kBT (i.e., Boltzmann constant×T(Kelvin)) at low pulse frequencies while operating at low/moderate driving B-field (milli-Tesla) strengths.

Moreover, EMF magnitude depends on the B-field flux area. Thus, in the in addition to pulse width, the summation of EMFs across multiple flux areas would need to be considered to estimate induced EMF dependent effects over an integral of theoretical rings of potential/conduction involving contiguous glycocalyx over multiple cell-contacts in tumor-cell monolayers: This is challenging to measure, where any one EMF across flux area A (estimated by A×dB/dt) induces forces on a given charged glycocalyx ring bounding area A to release tumor-cell proteases by that specific ring of cells. For charged glycocalyx across a cell monolayer, this can vary widely over the diameter of the entire well (on the order of several millimeters), making it difficult to estimate which “rings” contribute to the summed EMFs that drive net cell leak in the well. Conceptually, in real tumors this summation of EMFs may be favorable given the ability to simultaneously recruit charge over contiguous tumor-cell glycocalyces, and thus achieve wide spatial tumor stress effects over a 3-D tumor multicellular population. These considerations also give insight to aim in future studies to directly observe glycocalyx movement with each pulse possibly using advanced microscopy systems. This may facilitate estimates of the real energies involved in macroscopic electro-mechanical coupling in tumor multi-cellular systems.

More generally, while introducing brain tumor cell lines in the system, and with the above insights on the importance of Sia overexpression in the glycocalyx of a variety of brain tumors, how Lf-PMF protease-leak responses can depend on B-field strength, spatial characteristics, and manipulation of tumor glycocalyx Sia in the system was examined. In addition to greater magnitude effects by a higher intensity (T/sec) pulsing system (compare FIG. 1A to FIG. 1i), and the effects of spatial variation in B-field strength (high and low dB/dt in FIG. 2), mechanistic importance was found in noting that Lf-PMF effects are sensitive to Sia elimination. AUS sialidase is capable of digesting terminal Sia monosaccharides linked through any possible Sia glycosidic bond to terminal galactose (i.e., α2,3 or α2,6 linked Sia), or to Sia itself via α2,8 linked Sia-Sia linkages in polySia polymers. The latter are likely to play a role in expressing regions of high Sia density in the glycocalyx of SHSY or other brain or lung tumor cells (e.g., neuroendocrine tumors such as small cell lung cancer), where long Sia polymers are attached to unique tumor over-expressed membrane proteins such as neural cell adhesion molecule (NCAM) (Valentiner et al 2011). In this setting, Lf-PMF induced molecular motion of membrane core proteins heavily “decorated” with anionic Sia (or polySia) projecting into the tumor glycocalyx may result in EMF-driven membrane shear stress and leak dependent upon Sia over-expression. Indeed, Lf-PMF mediated protease leak from T98G and SHSY cell lines was sensitive to AUS pre-treatment of tumor cells immediately prior to the Lf-PMF stimulus, with significant post-AUS reductions in leak for both cell lines, and near-abrogation of leak in SHSY cells pre-treated with the enzyme (FIGS. 3A and B, FIG. 5A, graphs).

The tumor glycocalyx may be regarded as a glycan “canopy” above the tumor plasma membrane, with a particularly heavy composition of anionic glycans. The latter are anchored broadly to the membrane by over-expressed proteoglycan and/or mucin core proteins that project attached glycans (anionic glycosaminoglycan chains and/or Sia modifications, respectively) densely into the canopy. Pulsed EMFs may be able to interact with any “dominant” anionic glycan(s) in the canopy as whole, essentially as an inter-connected “slab.” The role that sulfated glycosaminoglycans like HS may play in transducing Lf-PMF pulses into membrane leak in A549 cells is supported by the fact that Lf-PMF induced leak is completely abrogated by pre-treating cells with heparinase (which destroys HS chains) before Lf-PMF exposure (FIG. 5B). It is notable that pre-treatment of A549 cells with AUS sialidase prior to Lf-PMF exposure was sufficient to inhibit Lf-PMF induced protease leak by˜70% (data not shown). While heparinase pre-treatment of A549 cells completely abrogates Lf-PMF mediated leak, AUS-mediated inhibition is appreciable, and shows that destroying even a non-dominant anionic glycan (i.e., Sia) in a tumor glycocalyx “dominated” by sulfated glycosaminoglycan chains as the major anionic species (as in A549 cells), may significantly alter the EMF mediated effect. The data herein demonstrates that Sia modified glycan species might interact in some way with HS glycosaminoglycans in the glycocalyx to mediate Lf-PMF effects. In this way, while HS is absolutely required for any Lf-PMF leak effect, loss of Sia also markedly inhibits the phenomenon: A simple biophysical concept that may reconcile this is that glycan modified membrane proteins (or lipids) do not respond independently to EMF forces, but rather the overexpressed glycans on such molecules contribute to a common anionic glycan “bulk” in the tumor glycocalyx (i.e., as part of a larger rigid structure). Thus, EMF-driven responses may depend in a synergistic way on lattice-like properties of both dominant HS and “interspersed” Sia in a common glycocalyx slab.

The data presented herein shows that species such as glycolipids (often overexpressed in brain tumors, with a high density of terminal Sia residues), hyaluronan (a sulfateless GAG polymer that is also anionic), or other species are involved, possibly through shared presence and interaction within the glycocalyx as a mechanical unit. Thus, other magneto-sensitive glycocalyx constituents may indirectly interact with overexpressed Sia residues or GAGs in a “cooperative” way, and with electro-mechanical sensitivity to chemical digestion of these glycan components in the implicated cell lines. Unique specific gangliosides, including subsets that are more heavily sialylated (e.g., GD3 and GD2) appear to be present in high quantities on T98G cells, as distinct from GDla and GM2 species that predominate on SHSY cells (Sorokin et al 2020; Dae et al 2009); however, the latter is characterized by heavier polySia expression, with numerous Sia residues polymerized on NCAM core proteins (Valentiner et al 2011). The latter may lead to unique magneto-sensitivity of SHSY cells. Along with GSL growth signaling in lipid rafts (Head et al 2014; Sasaki et al 2021), the sphingolipid acyl chains of GSLs can partially span the membrane to facilitate transmembrane events in the absence of transmembrane proteins (Head et al 2014). It is thus plausible to consider whether Lf-PMF repeated pulsing might alter or disrupt transmembrane events such as tumor growth signaling through unique mechano-transduction. Accordingly, the data presented herein shows implications on shifting to apoptotic signaling upon Lf-PMF induced ganglioside signaling-complex disruption

Mechanisms mediating Lf-PMF induced protease leak in tumor cells can include: (A) “molecular torque,” wherein transmission of EMF-induced force on glycosylated charged molecular termini in the tumor glycocalyx transduce torque about transmembrane points of core-protein attachment to the malignant plasma membrane via the core protein shafts serving as “lever arms.” (B) wave-like “rippling” of the glycocalyx slab over the malignant plasma membrane, and possibly associated membrane-shear from pulsed movement of the broadly attached slab as a unit. In some tumors, the latter may include hyaluronan (HA), a poly-anionic non-sulfated glycosaminoglycan, wherein HS binding to the CD44 receptor overexpressed on the tumor plasma membrane (Mitchell et al 2014) may create more “anchors” of the broad glycocalyx slab via “free-floating” HA in the glycocalyx mass as a whole, with further potential to contribute membrane stress (at points of CD44 membrane attachment) and leak upon Lf-PMF driven EMF engagement with the anionic mass as a whole. (C) Direct interactions with sub-membrane EMF-sensitive components, including cytoskeleton, not necessarily transduced via the glycocalyx. Intriguingly, beyond outward cellular protease leak, inward leak of exogenously added PI during Lf-PMF exposure alternatively demonstrated the Lf-PMF induced membrane leak under identical conditions. The magnitude of leak under the two methods in the proof-of-concept was similar (FIG. 4B), illustrating that induction-driven plasma membrane disruption/leak (rather than another mechanism leading to protease release into the exogenous environment) was operating as a result of the mechanism shown in FIG. 4A involving EMF induced forces on the tumor glycocalyx.

Independent of the magnitude of leak and potential mechanical stress in the immediate sub-plasma membrane region is the cytoplasmic induction of free radical and reactive oxygen species (ROS) that has been cited in a variety of post-pulsed-magnetic field experimental scenarios in distinct cell-systems (Wang et al 2017). An assessment of this under Lf-PMF pulsing conditions was conducted using model A549 tumor cells as a cell line in which low frequency PMFs or even pulsed electric fields have been examined for ROS behavior (Wang et al 2017; Wang et al 2018; Novickij et al 2022); and noting a unique variation in ROS signal post-exposure with a distinct early rise followed by modest reduction or “dip” in ROS signal after 30 min post stimulation in Lf-PMF exposed cells, after which the recovered cells paralleled control ROS conditions. This behavior at 30 to 60 min post-exposure appeared to be reproducible in repeated experiments, and showed the possibility of an initial ROS stimulation in Lf-PMF pulsed tumor cells that induces compensatory anti-oxidant systems that might dampen or inhibit ROS presence in the period between 30 min and 3 hr post exposure. This was consistent with literature using the same tumor cell type in low-frequency PMF platforms, where reductions in ROS relative to control are seen (Wang et al 2017), in contrast to higher-frequency or distinct longer-exposure conditions or in distinct tumor cell types. Whether these differences are influenced by distinct glycocalyx composition or correlate with associated glycocalyx-dependent leak remains to be explored, but the correlation of a unique ROS profile with Lf-PMF exposure is intriguing. While exogenous heparin has been shown to inhibit ROS production in distinct inflammatory cell types (Dandona et al 1999), the data presented herein examined native tumor cell-surface heparan sulfate (which is “tethered” to the membrane as a glycocalyx component in A549 tumor cells). Eliminating cell-surface heparan sulfate by pre-treating cells with heparinase before Lf-PMF exposure did not appear to change the ROS temporal profile following exposure (discussed above). So possibly the fairly modest changes in ROS production resulting from Lf-PMF pulsing under the reported conditions may function through additional mechanisms within the tumor cell that may be independent of glycocalyx-induced leak. Literature suggests highly variable ROS behavior in PMF-driven tumor cells, a variety of possible mechanisms, and active homeostatic compensatory pathways as well (Wang et al 2017; Reale et al 2014). ROS production may also be driven secondarily to magnetic field effects (e.g., following an immediate first step of membrane leak induced by EMF driven glycan-molecular torque and membrane stress), with subsequent effects on cell growth. Possibly, observing altered magnetic field effects after exhaustive addition of ROS scavengers to the system during Lf-PMF pulsing may further implicate a role played by ROS production in the observed effects (with some caution to any broader and/or toxic effects of excessively high scavengers themselves).

Mechanisms (A) and (B) considered above seem plausible in a variety of tumors given loss of leak effects immediately following highly specific digestion of cell-surface/glycocalyx glycans from the data of multiple experiments demonstrated here (FIG. 3B, FIG. 5A, and FIG. 5B). These mechanisms may disrupt the membrane at points of proteoglycan or Sia-rich glycoprotein attachment to the membrane (Pinho et al 2015; Chin-Hun Kuo et al 2018; Sarrazin et al 2011). Such motion may also impact other channels/gates for larger molecules to leak during pulsed EMF-driven glycocalyx motion. While the type of stress that this induces in cancer cells remains to be defined, some preliminary studies (that have not necessarily explored cell-surface mechanisms) do report alterations in tumor cell viability and/or apoptosis (Tofani et al 2001; Koh et al 2008; Omote et al 1990), but the conditions predicting these effects are undefined. Lf-PMFs may engage with a heavy interstitial glycan landscape that comprises tumor extracellular matrix: Assessing Lf-PMF effects on this matrix in the setting of whole in vivo preparations in future work may guide an understanding of secondary effects in facilitating tumor immunologic events (e.g., anti-tumor cytotoxic T cell access to tumor cell nests within tumor matrix, including tumor cell penetration by T cells after Lf-PMF exposures (Nie et al 2013; Nie et al 2013).

An entirely alternative model that can drive downstream biological effects from pulsed magnetic fields at relatively low energies would be a quantum spin model of radical pairs that may be operative. It involves magnetically induced changes in the spin of interacting electron pairs between singlet and triplet states that can drive reactions at otherwise energies even below the thermal background and has been implicated in a variety of cellular biochemical reactions that impact biological processes (Zadeh-Haghighi et al 2022). One convention that may implicate the possibility of “active” magneto-sensitive radical pairs in the system is the use of a second time-dependent magnetic field to modify or possibly even counter the effects of the original experimental field of interest (Hore et al 2016). The second field must have frequency that matches one of the frequencies with which radical pair(s) oscillate between singlet and triplet states in the primary experimental (usually static-) field. Moreover, if the system is “spin-coupled,” then one would expect there to be effects across those portions of the cells with atoms that have many electrons susceptible to spin-related effects (such as iron) and absent elsewhere. Cancer cells exhibit greater dependence on iron compared to normal cells (Chen et al 2019), and this could also be implicated in the differential effects.

Beyond carcinoma membrane biology and biophysics as well as the translational potential of incorporating such platforms into potential future tumor biomarker and therapeutic strategies, the transformative considerations of appropriately pulsed Lf-PMFs may be broad, opening a domain of biophysics that may harness pulsed magnetic fields to couple with unique membrane charge distributions in nature to selectively target the glycan surfaces of microbial pathogens or other biomolecules.

Lf-PMFs may impart via EMF induction forces on the unique glycocalyx of tumor cells. This results in membrane protease leak, which is sensitive to specific elimination of anionic glycan species such as sialic acid as a dominant glycan in model brain tumor cells or sulfated glycosaminoglycans as dominant glycans on model lung carcinoma cells. This may represent a “tunable” tumor-glycocalyx transduction mechanism via specific glycans depending on the malignant cell type; and provides further mechanistic understanding to Lf-PMF driven tumor-cell events that can lead to downstream tumor-directed cell stress in future translational strategies.

### Example 2

The use of oscillating magnetic fields to alter cancer cell growth has been described in biophysics, but the field strengths and/or frequencies to consistently induce cancer cell death or tumor regression in biological models remain a mystery. Recent studies showed that “pulsed” magnetic fields induce electromotive forces (EMFs) that engage with charged molecules that specifically enrich the glycocalyx of cancer-cell membranes (Ashdown et al 2020). This results in membrane leak and reduced tumor viability. Unlike cancer radiotherapy that targets high-energy ionizing radiation to S-phase DNA of rapidly dividing tumor cells, low frequency narrow pulse-width magnetic fields can be used to: (1) selectively couple with the cancer-cell glycocalyx to disrupt tumor membranes, and (2) inhibit viability of macroscopic as well as micro-metastatic tumor deposits in vivo over wide anatomic regions. Low-frequency pulsed magnetic fields are able to inhibit tumor growth in mice (Nie et al 2013; Tatarov et al 2011); and the discovery of cancer-cell glycocalyx charge properties that make cancer membranes uniquely susceptible to pulsed field disruption opens a door to new tumor-glycan biomarker development in addition to therapeutic translation. Beyond cancer, the transformative impact is broad, opening a domain of biophysics that may harness pulsed magnetic fields to couple with unique membrane charge distributions in nature to selectively target the unique glycan surface of neoplastic or even microbial pathogens.

In vivo efficacy and cell-based efficacy of pulsed magnetic fields in cell lines and a few mouse tumor models have been shown. The control of effects or knowledge on cell specificity, specific biophysical/membrane mechanism(s), and connection between cellular physiology and in vivo applications remains undefined. In general, in vitro and in vivo studies have demonstrated high heterogeneity, variability in platforms, and a lack of knowledge on how such fields may specifically affect cancer cells. However, the majority of studies, including in vivo whole-mouse platforms, show an inhibitory effect on tumor growth. In some cases, very low frequencies are used (Tatarov et al 2011; Koh et al 2008; de Seze et al 2000; Zhang et al 2002), and this appears to show efficacy in altering/inhibiting tumor cell proliferative characteristics (inhibit growth and/or augment apoptosis).

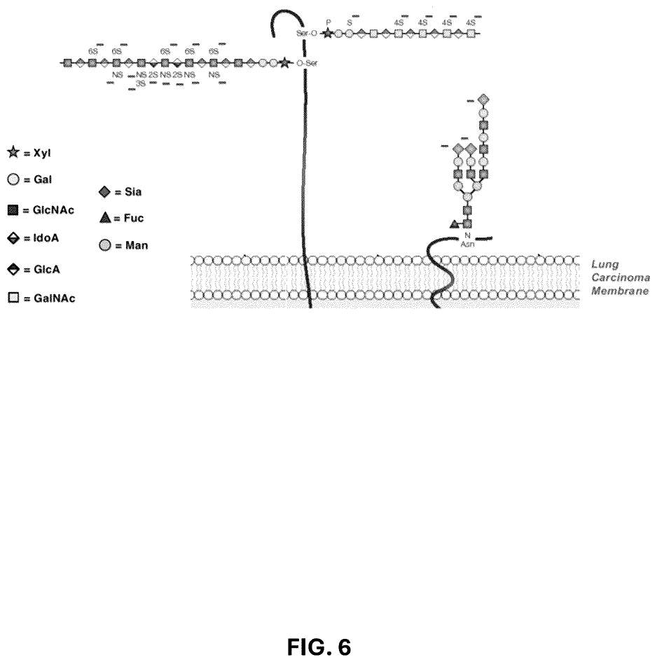

A new mediator for pulsed magnetic field induced leak in carcinoma cell membranes has been discovered. This is also by virtue of the fact that lung cancer cells and many other tumors over-express two types of heavily charged glycans as part of their glycocalyx: Sulfated glycosaminoglycans (such as heparan sulfate and chondroitin sulfate) and sialic acid, which is a monosaccharide containing an anionic single negatively charged oxygen atom (at physiologic pH) which decorates the termini of poly-antennary O-linked and N-linked glycans over-expressed on tumor-surface glycoproteins (Kang et al 2018; Fuster et al 2005; Nagaranjan et al 2018; Pinho et al 2015; Rodrigues et al 2018; Seidenfaden et al 2003). Local electromotive forces (EMFs) induced by pulsed magnetic fields may interact with the anionic glycans so as to torque them at their “tethering” points to the membrane. This may disrupt the membrane at the point of proteoglycan or sialic acid rich glycoprotein attachment to the membrane (Pinho et al 2015; Sarrazin et al 2011; Chin-Hun Kuo et al 2011). Such motion may also impact other channels/gates for larger molecules to leak during pulsed EMF-driven glycocalyx motion. While the type of stress that this induces in cancer cells remains to be defined, some preliminary studies (that have not necessarily explored cell-surface mechanisms) do report alterations in tumor cell viability and/or apoptosis (Koh et al 2008; Omote et al 1990), but the conditions predicting these effects are undefined. FIG. 4A and FIG. 6 illustrate how a pulsed magnetic field creates an EMF that may impose force on anionic glycan moieties (glycan sulfation and terminal sialic acid) that in turn can induce motion of core proteins that scaffold charged sugars within the glycocalyx (Fuster et al 2005).

This study reagrding lung cancer, and tobacco driven lung neoplasia: (1) Explores mechanisms through which unique tumor glycocalyx associated glycans mediate tumor cell leak and membrane stress on lung cancer cells; and reversal of the effects via glycan targeting (including exploration of possible augmented effects in tobacco-induced lung cancer cells as a result of a unique glycocalyx composition). (2) Studies the unique effect of pulsed magnetic fields (PMFs) on in vivo growth of human lung cancer cells in compatible mice, including tobacco-induced lung tumor cells grown in vivo following PMF exposure in culture or during real-time tumor growth in living/mouse hosts.

The unique properties of the tumor glycocalyx (cell-surface complex carbohydrate layer) (Chin-Hun Kuo et al 2018) are studied herein, examining how pulsed magnetic fields that selectively engage electrically with the glycocalyx of tobacco induced lung cancers may be especially susceptible to tumor membrane leak and impaired tumor viability, while comparing to effects on non tobacco associated lung tumors and normal lung cells. These studies may have positive impacts on developing new non-toxic and specific therapy for lung cancer as the leading cause of cancer death (Siegel et al 2022).

Oscillating magnetic fields driven by coil systems have inhibited tumor growth in mice (Tatarov et al 2011; Nie et al 2013). The cancer glycocalyx is rich in anionic complex carbohydrates (glycans) nested in a “canopy” ˜200 nm above the tumor cell membrane (Kang et al 2018; Pinho et al 2015). These are: (i) sulfated glycosaminoglycans such as heparan sulfate (HS) or chondroitin sulfate (CS); and/or (ii) sialic acid (SA), as poly-sialic acid (polySA) or overexpressed as charged terminal monosaccharides on tumor glycoproteins (Pinho et al 2015). High expression of such glycans augments metastatic and invasive potential, and so coupling pulsed-magnet induced tumor leak (and tumor lethality) to such glycocalyx properties may also selectively target highly invasive tumor cell subsets. EMFs generated by narrow pulse width low frequency pulsed magnetic fields (Lf-PMF) can: (1) engage with unique molecular charge on the cancer glycocalyx to disrupt tumor membrane integrity, and (2) inhibit tumor viability in vivo using human lung cancer cell xenografts in mice under real-time Lf-PMF exposures.

The results disclosed herein examine how tumor membrane disruption induced by pulsed EMF-driven glycocalyx stress elicits selective tumor-cell permeability, apoptotic signaling, and tumor-cell death. Additionally, the results provide guidance for the design of non-ionizing wave technology to selectively ablate tumor cell “sanctuaries” independent of chemical, cellular, or immunologic pathways to reach cancer-cell nests without the use of an “aimed beam” or toxic therapies.

Lf-PMF induced lung cancer cell glycocalyx motion, membrane leak, and viability will be assessed: Lung cancer cell monolayers bearing unique glycocalyx charge distributions will be treated, including tobacco-induced lung cancers, with low frequency (<50 Hz) pulsed fields (10-100 mTesla; pulse-width <100 μs) to generate leak-inducing EMFs on tumor-cells. Glycan composition (including HS/CS and SA expression) and study real time glycocalyx motion will be assessed using Lattice Light Sheet microscopy.

How pulsed magnetic fields affect growth of lung tumors in vivo will be assesed: The degree to which Lf-PMFs inhibit experimental tumor growth will be studied using human tobacco-induced lung cancer cell xenografts in immune-deficient (nu/nu) mouse hosts to allow modeling of human tumor growth. This will include assessing effects on in-vivo tumor growth signaling, and comparison to effects on the glycocalyx of non-tobacco associated lung tumor cells and normal control (non-tumor) cells.

Harnessing magnetic fields to alter cancer cell growth is an untapped arena that remains without an understanding of biophysical mechanisms that mediate tumor cell stress, behavior, and selective tumor-cell alterations (Tatarov et al 2011; Nie et al 2013). Innovation can be envisioned in the “pulsing” of magnetic fields that induce EMFs that can selectively engage with high-density molecular charge concentrated on glycans in the cancer membrane glycocalyx. Lung cancers and other malignancies are characterized by a high density of glycocalyx-associated charge conferred by anionic sulfated glycosaminoglycans such as HS or CS and/or the anionic sugar sialic acid (SA), commonly overexpressed by lung cancer cells and induced by tobacco exposure (Pinho et al 2015; Seidenfaden et al 2003; Vasseur et al 2012). The mechanism by which Lf-PMFs selectively engage the cancer glycocalyx will be explored, as real-time glycocalyx movement will be demonstrated as a result of EMF-driven force on the dense anionic layer. This can be correlated with tumor-membrane leak, disruption, and altered tumor cell viability. Despite regression with many lung cancer therapies, cancer often recurs locally, or with growth and death from micro-metastases (Dai et al 2013). Targeting unique properties of the lung cancer glycocalyx in a selective and “cell-cycle independent” approach using Lf-PMFs is innovative. Such fields may ultimately become an “Achilles heel” for field-exposed selectively targeted cancer cells. Studies show that pulsed-field EMFs can engage with charged tumor glycans to disrupt tumor membrane integrity (Ashdown et al 2020): Anti-tumor strategies that may expose whole-body regions to pulsed fields to selectively eradicate tumor cells. Novel strategies, including non-toxic adjuvant therapy to clear residual or micro-metastatic cancer cells are needed. As an innovative concept, applying pulsed fields in such settings may result in eradication of “visible and invisible” tumor, with potentially high future impact.

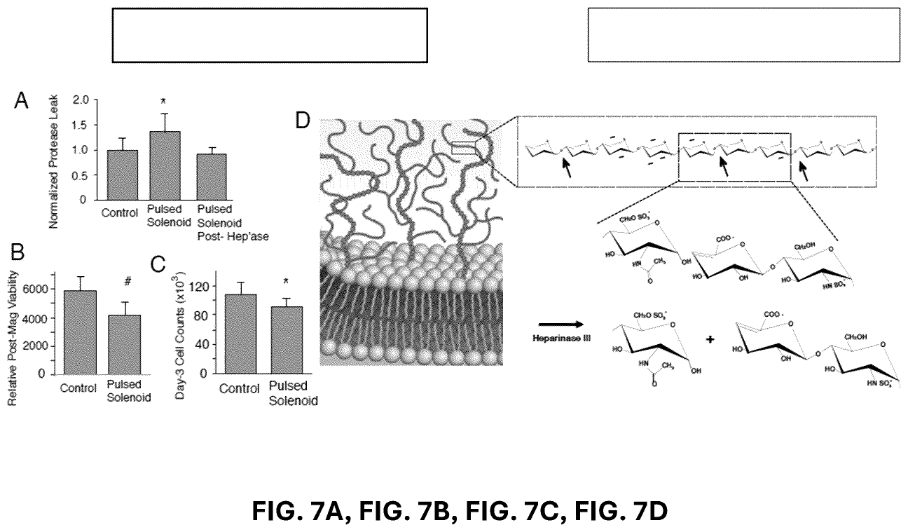

Lung cancer cell susceptibility to a low frequency pulsed magnetic field (Lf-PMF): Monolayers of lung cancer A549 cells were exposed to a solenoid emitting a pulsed magnetic field immediately below the monolayer plate, producing fields of 20 mT that included 5 min trains at frequency 50 Hz with a pulse-field duty-cycle rise time of approximately 10 msec. This short low energy Lf-PMF pulse-train was sufficient to induce tumor-cell membrane leak (measured by a commercial protease-leak assay) by monolayer tumor cells upon measurement immediately after exposure (FIG. 7A; middle bar). Strikingly, cells become insensitive to an identical Lf-PMF train exposure after digesting the cells with HS lyase (Heparinase III), which destroyed glycocalyx associated HS glycan chains (Alekseeva et al 2019) (FIG. 7A, right bar). HS proteoglycans are abundantly over-expressed on A549 and many other lung tumor cells (Ashdown et al 2020; Berry et al 1991). The pulsed field exposure also alters A549 tumor-cell viability, with additional (including published; Ashdown et al 2020) data that highlights a lack of leak and viability alteration on primary human lymphatic endothelial cells, as normal primary cells in the lung cancer microenvironment. Compared to control (non magnet exposed) A549 cells, Lf-PMF exposed A549 cells showed reduced viability at 4 hours following the exposure (FIG. 7B). Moreover, when Lf-PMF exposed A549 tumor cells were placed back into culture following a short 5-min exposure, cell-growth at 3 days was modestly but significantly inhibited compared to that of control cells (FIG. 7D). In FIG. 7D, a HS glycan dominant glycocalyx on the tumor surface (e.g., A549 cells) was observed, with heparin lyase enzyme treatment that destroyed glycan chains mediating the leak effect, and loss of Lf-PMF sensitivity following enzyme treatment (as in FIG. 7A).

Induction of Lf-PMF leak and reduced viability in sialic acid rich tumor cells: SA, a negatively charged sugar that heavily modifies branched glycan termini of complex glycoproteins on the cancer glycocalyx (Pinho et al 2015; Rodrigues et al 2018), is uniquely over-expressed on a variety of tumor cells. These include small cell lung cancer cells (Gong et al 2017) and possibly broad expression in non-small cell lung cancers, with SA-rich glycocalyx induced via exposure to tobacco and upon neoplastic transformation (Vasseur et al 2012). Brain tumor model cells that overexpress SA can be used to study the sensitivity of tumors expressing high glycocalyx SA to Lf-PMFs, with a rationale that heavy anionic SA on tumor glycoproteins may induce membrane stress by Lf-PMF driven EMFs on brain tumor cells. High SA density is due to (i) High SA levels on the tips of branched terminal glycans on overexpressed tumor mucins, glycoproteins, or glycolipids, or (ii) dense polysialic (poly-Sia) expression (e.g., on neural cell adhesion molecule; NCAM) on a variety of glio- and neuro-blastoma cells (Amoureux et al 2010; Scheer et al 2020), small cell lung cancer, and possibly upon tobacco driven transformation in squamous non-small cell lung cancer pathogenesis (Nguyen et al 2000).

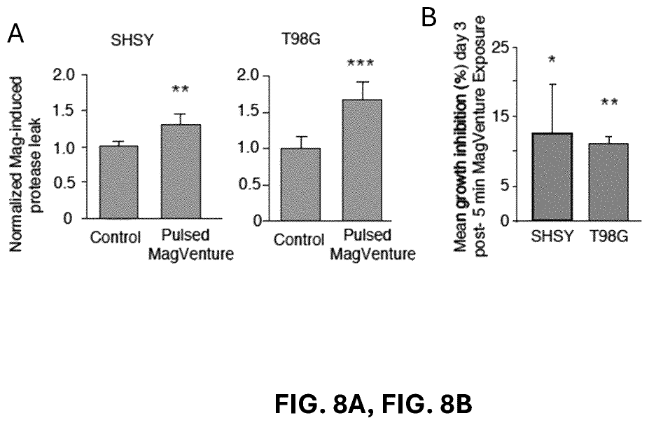

A neuroblastoma line, SHSY (SH-SY5Y) known to express polySia (Valentiner et al 2011) was tested in a pulsed solenoid system (20 mT amplitude; 10 msec dB/dt duty-cycle; 50 Hz) that was previously used in early Lf-PMF leak studies on lung and breast tumor cells (Ashdown et al 2020). A leak was detected by SHSY monolayers exposed to this system under identical conditions (FIG. 1A, right graph). A Lf-PMF system was employed with higher amplitude over a narrower pulse width (pulse over 70 ocsec with dB/dt˜8 kT/sec; 15 Hz trains). This MagVenture unit is used for clinical Transcranial Magnetic Stimulation (TMS), and includes a “rat coil” platform generating dB/dt across a trough that can be laterally oriented with a 96-W plate inserted in the field with fluxing B-lines perpendicular to the cells (Drakaki et al 2022). This resulted in greater magnitude effects (FIG. 8A), and it is known that T98G also overexpresses SA from prior studies (Cuello et al 2020). A late (day 3 post exposure) inhibition of tumor cell viability when cells are placed back in culture following a 5-min Lf-PMF exposure was also noted (FIG. 8B).

Reduction in Lf-PMF tumor membrane leak by eliminating glycocalyx SA: The effects of Lf-PMF in the presence/absence of enzymatic SA digestion from the tumor membrane was examined. Leak induced by Lf-PMF exposure of SHSY monolayers to pulsed-field fluxing using a MagVenture unit (dB/dt˜8 kT/sec; 15 Hz train for 5 min) was entirely abrogated by pre-treating cells with AUS sialidase (FIG. 3A). A significant and reproducible reduction in Lf-PMF induced membrane leak was achieved by AUS sialidase pre-treatment in T98G cells as well as SHSY tumor cells in multiple trials (FIG. 3B). As noted in FIG. 3B, the ability of sialidase treatment to inhibit Lf-PMF induced leak was nearly complete in SHSY cells (mean˜80% inhibition, FIG. 3B right bar), evidencing the importance of SA on SHSY cells in mediating the induced-EMF associated protease leak. This can be due to the polySia modification that is known to exist for especially heavy SA expression on the surface of SHSY cells, while T98G cells likely have a lower SA density on the surface, but sufficient SA modifications to achieve partial (mean˜20%) inhibition in Lf-PMF leak following enzymatic clearance (FIG. 3B, left bar). FIG. 3C illustrates the effect of sialidase treatment on eliminating terminal sialic acid modifications that richly decorate carcinoma cell glycoproteins (including mucins), heavily expressed as SA capped branched complex glycans in the glycocalyx (as illustrated here) or as polySia SA polymers linked to cell adhesion molecule proteins. This was carried out to examine the extent to which AUS sialidase inhibits or abrogates the Lf-PMF induced membrane leak in SHSY and T98G model tumor cells.

Relative sensitivity of Lf-PMF induced leak in multiple tumor cell lines using MagVenture coil: An additional human glioblastoma cell line (A172) with lesser expression of SA on the cell surface was examined (as determined by flow cytometry of cell-surface SA using fluorescent SA-binding lectin probes pre/post digestion with AUS) as well as multiple tumor cell lines (as shown in FIG. 2) for protease leak following a MagVenture Lf-PMF exposure for 5 min to two distinct dB/dt flux strengths, with clear leak responses as well as dose-response effects shown for high versus low flux strengths. It is notable that A549 lung cancer cells showed marked leak relative to brain tumor lines, while all tumor lines were sensitive; and primary dendritic immune cells (as a normal non-neoplastic cell line) showed no augmented leak to the Lf-PMF exposure (if anything, there was protection from leak), paralleling findings of an inert state (or lack of leak response) by primary lymphatic endothelial cells to Lf-PMFs (Ashdown et al 2020). Squamous lung cancer cell lines (including tobacco-exposed) will be assessed similarly for pulsed-field sensitivity, in addition to small cell lung cancer and reference A549 cells.

Data herein shows Lf-PMF interactions with glycocalyx constituents over-expressed in lung cancer: Sulfated glycosaminoglycans and SA-modifications that “cap” glycoproteins attached to the tumor membrane. Such modifications are known and predicted on tobacco-induced lung carcinoma subtypes that remain unexplored in this area: These include small cell and squamous cell lung carcinomas (Vasseur et al 2012; Gong et al 2017; Nguyen et al 200). Proposed studies include Lf-PMF lung carcinoma targeting approaches, assessment of glycan-mediated leak and viability/growth studies with parallel glycocalyx composition assessments, and establishment of targeting susceptibility.

Lung cancer monolayers bearing characteristic glycocalyx charged-glycans will be treated with Lf-PMF induced EMFs of sufficient strength to induce tumor membrane leak in culture. This will include real-time assessments of Lf-PMF driven molecular motion through advanced microscopy systems, and assessment of glycocalyx composition for each line (noting associated cell line tobacco exposure data).

Membrane leak and viability post-Lf-PMF in lung squamous and small cell cancer lines: Model cancer cell lines will be utilized A549 (ATCC) as a typical glycosaminoglycan overexpressing lung cancer (adenocarcinoma) line; NCI-H69, as a typical polySA expressing small cell lung cancer line; and three squamous cell lines that developed in the presence or absence of tobacco exposure (ATCC squamous lung cancer lines: NCI H1703, from heavy smoker; H1869 from heavy smoker; H2170 from non-smoker). The squamous lines are also selected for possible differential glycan/SA expression among tobacco-exposed subsets. As controls, primary lung lymphatic endothelial cells (HMVEC-LLy; Lonza) as non-cancer primary cells from the cancer microenvironment will be used (Ashdown et al 2020). Tumor membrane disruption will be assessed via luminescence-based protease leak assays (Promega), to quantify leak into the medium after 5 min pulse-field exposures of cell monolayers in the MagVenture coil unit (FIG. 2). Tumor cell viability will be quantified following daily 5 minute field exposures over 5 days, comparing to non-pulsed control cells. FIG. 1C illustrates the Lf-PMF concept and parameters for treating cell monolayers using a high flux (high dB/dt with narrow (dt) pulse width) MagVenture system.

Glycocalyx composition and correlation with Lf-PMF exposure effects: Cell lines will be examined for SA content as well as HS and CS content through standard glycan analysis: low cytometry with lectin markers for terminal SA assessment and SA reduction with AUS sialidase treatment. Glycocalyx composition will be correlated with Lf-PMF leak magnitude (FIG. 2) and with tobacco status, with hypothetical increased SA expression in tobacco-exposed squamous lines. FIG. 3D shows glycan detection methods using fluorescent lectin-based flow cytometry to quantify tumor cell surface reactivity and reduction in SA following AUS sialidase treatment of cultured SHSY cells (confirming reagent enzymatic activity as well as SA expression changes that correlate with inhibition in Lf-PMF induced leak upon AUS treatment of SHSY cells, as in FIG. 3A-3B).

Advanced microscopy systems to assess real-time Lf-PMF glycocalyx molecular motion: The preliminary functional studies establish conditions for which advanced microscopy will be used to directly visualize whether low frequency pulses with identical pulse-width and magnetic strength (mT) characteristics will be capable of inducing a pulsed-motion of the cancer cell glycocalyx (with comparison to that of non-cancer HMVEC-LLy cells under identical conditions). A lattice light sheet microscopy will be used to examine for such motion directly Digital high speed holographic microscopy will be utilized to capture tumor cell-membrane deflection under high-speed image acquisition during magnetic pulsing. These techniques will provide pulse-field dynamic tumor-membrane response data that will be critical for the growth of future mechanistic studies to optimize selective EMF-driven effects on malignant plasma membranes bearing glycocalyces with unique charge distributions.

Squamous and small cell lung cancer lines show marked protease leak and inhibited viability responses to Lf-PMF exposures, while molecular motion of the glycocalyx detected in phase with real-time field pulsing under advanced microscopy should be relatively straight forward. Alternatively scanning electron microscopy can be utilized to detect membrane “rippling” as it depends on glycan presence or absence (post-enzymatic digestion) upon Lf-PMF exposure.

The effects of Lf-PMF exposure on the growth and survival signaling properties of model human lung tumors will be assessed in vivo. This will include studies on low frequency pulsed-field effects on ex vivo (pre-implantation) and in vivo real time tumor exposure to external pulsed fields to examine inhibition of experimental tumor growth using human tobacco-induced lung cancer cell xenografts in immune-deficient (nu/nu) mouse hosts to allow modeling of human tumor growth. This will also include assessment of effects on in-vivo tumor growth signaling with comparison to control cells that include non-tobacco associated lung tumor cells and normal host cells in the Lf-PMF exposed field. Tumor growth inhibition may result from Lf-PMF exposure will be robust among lung cancer cell lines, and correlate with SA expression, HS/CS expression, and with especially robust effects in small-cell and squamous tumor lines dominated by tobacco exposure.

Lung tumor growth in vivo and Lf-PMF exposure including pilot tobacco-induction considerations: The effect of Lf-PMF exposure on model subcutaneous tumors grown as human lung cancer cell xenografts in immunocompromised (nu/nu) mice to (allow growth without rejection of human cells). Early concept studies will focus on (i) effects of real-time ex-vivo (pre-tumor inoculation) exposure of tumor cells to Lf-PMF for 5 min (rationale from cell-based preliminary data) on early tumor-take and growth on the nu/nu mouse background; and (ii) effects of in-vivo tumor exposure to external Lf-PMF versus control (sham) with daily pulsed field exposure after tumor inoculation (daily 5 min cross-tumor exposure using rat coil holding anesthetized mouse for the exposure). A549 cells, small cell NCI-H69 lung cancer cells, and 3 squamous cell lines (H1703 and H1869 from a tobacco-exposed human source; and H2170 from a non-smoker) will be used in separate experiments to assess effects of Lf-PMF exposure on early in vivo tumor growth. Tumor studies will be carried out for set time as an endpoint (typically 2-3 weeks for subcutaneous tumors), with mouse sacrifice and necropsy for all tumors at the time endpoint to measure tumor size characteristics, flow cytometry to measure tumor-cell apoptosis and glycocalyx properties, and histology.

Growth and apoptosis signaling in Lf-PMF exposed tumor cells: Following mouse sacrifice and tumor harvesting in IIA tumor growth studies, growth signaling will be assessed in ex vivo purified tumor cells (Miltenyi tumor-cell purification kit), comparing mitogen responses by tumor cells from Lf-PMF treated tumors to that of control (non-exposed) tumors. Mitogen growth signaling will be examined by western blotting (Erk and JAK phosphorylation), and tumor cell Annexin-V antibody labeling will be used to detect apoptotic cells by flow cytometry. Inhibition of mitogen signaling and augmented apoptosis signaling in pulsed-field treated tumor cells would suggest that treating tumors with Lf-PMFs inhibits tumor growth signaling and/or promotes cell-death pathway induction. Hypothetical heightened sensitivity to these effects will be assessed in tobacco-induced lung cancers while correlating with SA and HS/CS glycan expression on the cell surface of each tumor subtype.

In vivo tumor growth will be inhibited by Lf-PMF exposure, with the more pre-translational experimental platform examining early tumor growth during daily short external Lf-PMF tumor exposures in real-time in the living animal; and tobacco-exposed lines would be expected to show greater inhibition in tumor growth due to heavier expected SA expression (characterized biochemically in glycocalyx composition analyses). Extended Lf-PMF exposure periods will be tested in the tumor-bearing animal for a time-dose response. Alternatively, dB/dt (flux magnitude) may be increased on the MagVenture unit. As a sensitive measure in tumors that show minimal differences between Lf-PMF and control exposures despite the above adjustments, flow cytometry and viability markers can be used to co-label SA-expressing cells (using fluorescent SA-binding lectins) with viability (e.g., propidium iodide or zombie viability) reagents to assess magnet effects on viability of high-SA versus low-SA expressing tumor cell subsets. (Labeling of HS with 10E4 clone antibody (Lo et al 2011) is a sensitive method for sulfation-detection, and similar high/low 10E4 assessments with vital-dye co-labeling may be used for HS charge density in the glycocalyx).

For each tumor cell line, 5 measurements under each condition are needed to assess 20% differences in tumor-cell leak, with adjustment upon initial data analyses. Attention will be paid to effects related to mouse gender. Means will be compared by student's T-test, or by two-way ANOVA with appropriate post-hoc analysis depending on data distribution (PRISM software). Mouse studies will likely require n=6 mice per condition (e.g., Lf-PMF vs control) to achieve significant experimental differences in tumors between groups. Males and female mice will be included in studies to pay attention to sex as a potential variable. Significance will be based on P value of 0.05.

### Example 3

Altering cancer cell growth with magnetic fields has been reported in biophysics, but the field strength and frequencies to inhibit tumor cell behavior or growth remain a mystery (Koh et al 2008; Nie et al 2013; Williams et al 2001; Tatarov et al 2011). It was recently discovered that “pulsed” magnetic fields induce electromotive forces (EMFs) that appear to engage with molecular charge on the cancer cell's membrane glycocalyx, driving membrane leak and reduced tumor viability in a simple model. From these studies, such fields (including testing of narrow magnetic pulse widths) may selectively disrupt tumor cell membranes and viability in a typical spectrum of tumor glycocalyx compositions (Ashdown et al 2020).

Unlike radiotherapy that targets ionizing radiation to rapidly dividing tumor cells, pulsed magnetic fields may: (1) engage with a charge-rich glycocalyx (glycan layer) on human lung and brain tumor cells to disrupt tumor membrane integrity and viability, and (2) selectively inhibit the growth potential of tumor cells in vivo. Oscillating magnetic (“B1 field”) pulses driven by magnetic coil systems can inhibit tumor growth in mice (Nie et al 2013; Tatarov et al 2011). Discovering glycocalyx properties that make cancer cells uniquely susceptible to pulsed B1 fields opens a door to therapeutic translation. The cancer glycocalyx is rich in anionic glycans nested in a “canopy” ˜200 nm above the cell membrane (Kang et al 2018; Pinho et al 2015). These are: (i) sulfated glycosaminoglycans (GAGs) such as heparan sulfate and/or (ii) sialic acid (SA), as poly-SA or over-expressed as a terminal sugar on tumor glycoproteins (Pinho et al 2015).

The potential impact of this work may be high, harnessing biophysics to target a unique “Achilles heel” of the cancer cell. Studies herein will enable the design of trans-tissue pulsed B1 fields as novel strategies to selectively ablate tumor cells and micro-metastases. Pilot data showed that delivering low power, pulsed magnetic fields (<20 mT; pulse width<20 ms) at low frequency to cancer cell monolayers disrupts membrane integrity and cell viability. Strikingly, enzymatic GAG elimination on the cancer cell's glycocalyx rendered the cells insensitive to pulsed field membrane disruption. The same fields did not affect normal (non-cancer) vascular cells (Ashdown et al 2020). Pulse frequency may not be critical, as curiously low frequencies may achieve tumor inhibitory effects (Novikov et al 2009; Zhang et al 2002): Rather, narrowing the pulse (dB/dt) rise time may be the functional key to EMFs with potent selective tumor-membrane disruptive effects.

The effects of pulsed magnetic fields with a narrowing pulse width will be studied to assess the sensitivity of lung cancer and brain glioblastoma cell lines (with typically heavy glycocalyx GAG and poly-SA expression, respectively) to tumor membrane disruption/leak and altered cell-viability. Tumor cells may demonstrate high vulnerability to pulsed-field driven membrane disruption with greater magnitude as magnetic pulse-width is narrowed, and induced EMFs are increased. The most vulnerable tumor lines will be used to explore selectivity of the pulsed fields against tumor cells in co-culture systems using differential fluorescence-tagged fibroblasts mixed with tumor cells. B1 pulsed fields may selectively inhibit viability of co-cultured tumor cells over extended periods of pulse-field treatment in culture.

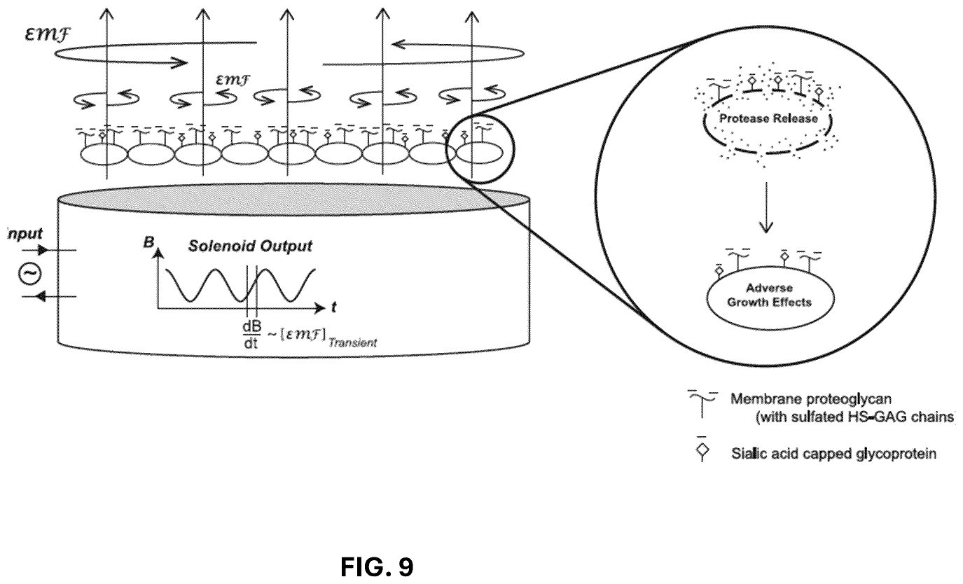

Responses of lung- and brain tumor cells to narrow-pulse B1 fields: Cultured tumor cells bearing characteristic glycocalyx charged-glycans (lung cancer cells overexpressing heparan- or chondroitin sulfate GAGs and glioblastoma cells overexpressing poly-SA) will be treated with variations in B1 field magnitude and pulse-width to generate EMFs with sufficient strength to achieve tumor membrane leak over sub-millimeter (micron scale) dimensions on tumor monolayers. Tumor membrane disruption will be assessed via luminescence-based protease leak assays (Promega), quantifying leak into the medium after 5 min pulse-field exposure of tumor monolayers using assays (Ashdown et al 2020). Dose responses to ultra-low frequency (1-50 Hz) pulses up to 100 mT will be quantified. Tumor cell viability will also be studied in the setting of 5 min pulse-field exposure periods, with daily viability assays over 5 days, comparing viability to that of control (non-exposed) cells. FIG. 9 illustrates concepts and rationale.

Pulsed-field tumor selectivity in co-culture: To effectively test the tumor-cell selectivity of narrow-width pulsed fields under a unique in-vitro physiologic co-culture context with high stringency, Mixed culture studies will be completed using quantum-dot (Qdot)(Wylie et al 2007) nanocrystal green-fluorescent (Qtracker 525; ThermoFisher) loaded fibroblasts mixed 1:1 at culture initiation with red-fluorescent (Qtracker 625) loaded tumor cells. In such assays, tumor cell lines with highest membrane disruption achieved in preliminary pulsed-field tumor screening studies will be used, exposing cells to twice-daily 5 min pulsed-field exposures (guided from preliminary published viability effects with as little as 5 min pulsed-field exposure of A549 tumor cells (Ashdown et al 2020)), and measuring red/green fluorescence of total well-harvested cell populations using flow cytometry on daily harvests during a 7 day exposure period. Apoptosis and viability of green versus red (tumor) labeled cells will also be examined.

In early proof-of-concept, the effects of B1 pulsed field treatment of tumor cells may be assessed during early growth in a tumor microenvironment through post-pulsed-field in vivo inoculation, and assessing the impact on early solid-tumor formation. Human lung cancer and glioblastoma cells may be uniquely sensitive to glycocalyx-mediated membrane disruption, resulting in altered proliferation and inhibited solid-tumor growth in vivo. Parallel ex-vivo tumor-cell apoptosis assays will be conducted, which many show that pre-inoculation pulsed-field treatment results in greater tumor apoptosis.

Effects of in vitro pulsed-field treatment on in-vivo growth of tumor cells: In exploratory in vivo studies, A549 tumor cells will be treated with B1 pulsed-fields (×5 min) versus control non-exposed cells (Ashdown et al 2020) harvested from culture, loaded with Qtracker 525 nanoprobes (for green-fluorescent live imaging), and inoculated subcutaneously for tumor growth in nude (nu/nu) mice. Thus, the effects of pulse-field pre-treatment on real-time early human tumor-cell growth via fluorescence imaging/tracking will be studied. Specifically, daily quantitative fluorescence images will be acquired from right versus left hindquarters of mice inoculated with pulse-field treated tumor cells (right flank) versus control tumor cells (left flank) at day 0. Following daily image acquisition for 7 days, tumor growth curves will be analyzed from right-versus left images to quantify growth inhibition resulting from the pulsed-field exposure.

Apoptosis in tumor cells grown in vivo following a single B1 pulse-field exposure: Upon sacrificing mice at day 7 post tumor inoculation above, with tumors established using B1 pulse-field treated cells (right hindquarter) versus control tumor cells (left hindquarter), right versus left respective tumors from mice will dissected, collagenase-digested, and labeled tumor digests with phycoerythrin (PE) tagged Annexin-V antibodies to detect apoptotic cells by flow cytometry (Crowley et al 2016). Since tumor cells bear green (Qtracker 525) label, double-positive (PE+/Qtracker+) cells sorted by flow cytometry will be used to quantify tumor cell specific apoptosis, reporting % double positive tumor cells (indexed to total tumor cell population) in right-flank (pulse-field exposed) tumors compared to that of left-flank (control) tumors. A finding of greater mean % double-positive tumor cells from B1 pulse-field exposed tumor cells would suggest that pre-treatment with B1 pulsed fields results in tumor-cell apoptosis in vivo. This will provide rationale for expanding studies to assess tumor-cell apoptosis/death using similar B1 pulse fields to treat established tumors in mice through external B1 magnetic coil systems.

Based on published work and sample size analysis, fit is estimated that for each tumor cell line, approximately 5 measurements under each condition are needed to assess 20% differences in tumor-cell leak. Data will be analyzed by either the student's T-test in simple comparisons of means, or by two-way ANOVA with appropriate post-hoc analysis depending on the distribution of data using PRISM software. Statistical significance will be based on P values of 0.05.

### Example 4

An array of published cell-based and small animal studies have demonstrated a variety of exposures of cancer cells or experimental carcinomas to electromagnetic (EM) wave platforms that are non-ionizing and non-thermal. Overall effects appear to be inhibitory, inducing cancer cell stress or death as well as inhibition in tumor growth in experimental models. A variety of physical input variables, including discrete frequencies, amplitudes, and exposure times, have been tested, but drawing methodologic rationale and mechanistic conclusions across studies is challenging. Nevertheless, outputs such as tumor cytotoxicity, apoptosis, tumor membrane electroporation and leak, and reactive oxygen species generation are intriguing. Early EM platforms in humans employ pulsed electric fields applied either externally or using interventional tumor contact to induce tumor cell electroporation with stromal, vascular, and immunologic sparing. It is also possible that direct or external exposures to non-thermal EM waves or pulsed magnetic fields may generate electromotive forces to engage with unique tumor cell properties, including tumor glycocalyx to induce carcinoma membrane disruption and stress, providing novel avenues to augment tumor antigen release, cross-presentation by tumor-resident immune cells, and anti-tumor immunity. Integration with existing checkpoint inhibitor strategies to boost immunotherapeutic effects in carcinomas may also emerge as a broadly effective strategy, but little has been considered or tested in this area. Unlike the use of chemo/radiation and/or targeted therapies in cancer, EM platforms may allow for the survival of tumor-associated immunologic cells, including naïve and sensitized anti-tumor T cells. Moreover, EM-induced cancer cell stress and apoptosis may potentiate endogenous tumor antigen-specific anti-tumor immunity. Clinical studies examining a few of these combined EM-platform approaches are in their infancy, and a greater thrust in research (including basic, clinical, and translational work) in understanding how EM platforms may integrate with immunotherapy will be critical in driving advances in cancer outcomes under this promising combination.

In the setting of carcinomas, a variety of questions, including the effects on heterogeneous carcinoma sub-clones that may have unique antigenic make-up or landscapes or the effects on tumor cells with distinct propensities to invade or metastasize. There may also be unique susceptibility of tumor mitochondria to the generation of reactive oxygen species or apoptosis induction (or their plasma membranes to electroporation phenomena). Cancer stem versus non-stem cells may also be differentially affected. All of these may depend on which EM platform is applied in any given exposure.

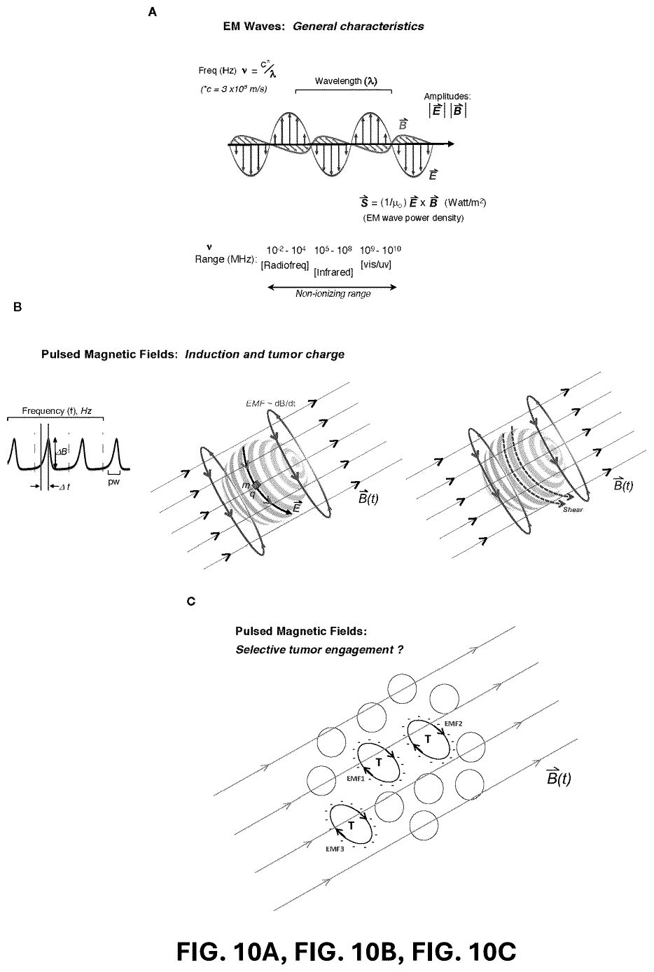

An under-reported variable in empirical tumor-model exposures to EM platforms is the shape of the duty cycle of the applied EM wave or pulsed/oscillating magnetic field. This parameter may be involved in the transduction of tumor cellular and molecular functions in response to the EM stimulus, including metabolic or growth-related stress. For a PMF approach, the rise or slope of each pulse may critically determine the degree of mechanistic coupling to a uniquely susceptible cellular element in malignant cells. In physical terms, the rate of rise in a magnetic pulse or oscillation (i.e., its “sharpness”) is conveyed as dB/dt (Xu et al 2021; Mills et al 2000; Novickij et al 2017). The EMF induced by that particular period of rise to the maximum amplitude may be more impactful on unique tumor cellular features (e.g., abnormally expressed molecular charge on the plasma membrane, microtubular features, or the state of chromatin). Specifically, in a classical consideration of Faraday's principle, it may be proposed that the rate of change in the magnetic flux density (dB/dt across area A) induces an EMF bounding area A, which can uniquely couple with charge-dominated tumor cell-specific elements (Mills et al 2000; Chin-Hun Kuo et al 2018; Johns et al 2023). FIG. 10 illustrates both the classical EM wave (FIG. 10A) and the PMF and how induced EMFs via the PMF approach may engage selectively with charged elements unique to tumor cells (single or in a contiguous colony) while sparing non-tumor cells or tissues. Such EMFs may engage with distinctly charged tumor membranes to induce molecular torque and shear forces along with cancer cell stress, with the example in FIG. 10B describing pulse train characteristics such as amplitude, frequency/period, duty cycle, rise time, and pulse width (top) and how induced EMFs may drive membrane shear by engaging tumor-overexpressed molecular charge and mass distributions. While a uniquely charged tumor plasma membrane is used in the illustration, the pulsed field may disrupt any number of candidate tumor-unique structures or states, including membrane integrity, microtubule or spindle function, chromatin integrity, or the generation of ROS in parallel to apoptosis as a direct or downstream result of tumor mitochondrial disruption. These may be sensitive to varying distinct pulse characteristics (e.g., rise time), and distinct tumor “nests” may be distinctly stressed through independent EMF effects (FIG. 10C).

The shape of a pulse delivered over the “duty cycle” is characterized by rise time as well as the pulse width (time over which the field is pulsed; FIG. 10B), which can be far less than the period of pulse delivery or oscillatory rate (Novickij et al 2017). Therefore, a low-frequency PMF that effectively couples (e.g., with substantial mechanical response) to a unique tumor cellular feature such as a highly charged tumor glycocalyx may “oscillate” at either 10 Hz or 10 kHz; however, the duty cycle and dB/dt may be designed identically for these two frequencies (for example, dB/dt delivered as 10 μT over 20 μs in both cases), wherein the efficacy lies in the duty cycle rise time over 20 μs rather than the PMF frequency (10 Hz or 10 kHz) per se. Thus, relatively low pulse amplitudes may be used to affect tumor-characteristic properties so long as “dt” is very short (i.e., narrow pulse width), and thus the independent parameters of dBmax (amplitude) and “dt” (effectively, the period of the duty cycle) should be separately reported. As another example, independent of frequency, if the period of a pulsed 50 mT B-field can deliver pulses over a 50-μs duty cycle (i.e., rise time <50 μs), the reporting of “a PMF delivered at dB/dt=1.0 kT/s” without information on the pulse-width parameters does not provide sufficient information. A PMF of 500 mT delivered over 500 μs could also be reported as “1.0 kT/s”, although such an exposure may be critically different than the former (possibly with markedly different biological effects), given the pulse delivery over a 10-fold narrower duty cycle in the former. Engagement of the induced EMF with sub-cellular structures, or even “confluent” cellular elements such as the tumor plasma membrane glycocalyx (e.g., over a microsphere of thousands of tumor cells), may critically depend on the very narrow pulse width, even if the B-field amplitude is much lower and independent of the PMF pulse-train frequency. [00231]“Pulsing” an EMF can have significant effects at ELFs, where the single pulse can be delivered at narrow pulse widths with relatively low energy (mT range) to cancer cell systems to perturb membrane activity. A mechanistic consideration regarding how the latter is affected in experimental tumor systems under external ELF-EMF generation involves the perturbation of ion fluxes that maintain a relatively depolarized state in resting cancer cells, and the effects that this has in driving metabolic thermal-generating responses that further stress relatively extreme non-equilibrium thermal state between the tumor cell and its environment due to greater metabolic activity (Bergandi et al 2022; Lucia et al 2020).

In an approach to an EM wave inducing tumor cell-specific resonance and stress, one may design a suitable “driving” frequency such that a full wavelength (oscillatory period) envelopes the tumor-cell diameter (including the richly anionic glycocalyx) or “fitting” a uniquely charged tumor organelle of interest in one cycle (41). With an EM wave as a pulsed electromagnetic field (PEMF), the uniquely charged tumor cell surface or even a unique organelle (tumor mitochondrion or S-phase nuclear chromatin domain) may retain unique characteristics that give it much greater charge than that of neighboring stromal cells. With a tumor cell as a target (diameters in a 10-30-μm range), a “driving” frequency in the THz range may achieve unique “capture” and tumor-cell resonance since that size is approximated by the incident EM wavelength (y=c/v, where c=3×108 m/s and v is in THz range), drawing analogy to classical mechanical wave dynamics (41): testing/tuning yin that range may achieve optimal “resonant” responses. Alternatively, one may capture a “clump” of tumor cell diameters with a confluent, shared glycocalyx “slab” at a frequency that is 10-fold lower (with y in the 100-300-μm range). Unique resonant EMF “capture” without effects on neighboring stromal cells may be possible since the surface of many tumor cells in vivo may be distinguished from stromal cells by virtue of a more highly charged glycocalyx (Chin-Hun Kuo et al 2018; Pinho et al 2015; Johns et al 2023). Additionally, while stromal cells may have lesser degrees of the same anionic glycans on their physiologic glycocalyces, the quest maybe to engage the glycocalyx with “driving” EMFs for even short mechanical capture periods against a mechanically distinct surround, regardless of whether one achieves full sustained mechanical resonance of the whole tumor cell or intracellular organelle under the pulsed driving force. Other variables to consider in any classical dynamics resonance approach include viscosity (gamma) or how the “spring” characteristics of surrounding tissue affect Q (quality factor) for any given tumor cell.

In another independent application of resonance, dedicated ELF-induced EMFs using coil-generated pulses in the <10-Hz range could be applied to 2D and 3D spheroid tumor cells to generate resonance with thermal dissipation that resembles or models according to resistor/capacitor “RC” circuit type behavior (39). This was achieved with the application of specific ELFs that could optimally drive responses that not only inhibited cancer cell growth but also generated ion fluxes (current) and dissipated heat. To maintain homeostasis, downstream mitochondrial responses (coupled and uncoupled activity with ATP production) appear to be boosted upon achieving this “thermal resonant frequency”.

Induction of EP by various modalities has been achieved under direct tumor contact with probes (pulsing range of 1 kHz-1 MHz). Cell death by irreversible electroporation (IRE) occurs by varying degrees via apoptosis and necrosis/necroptosis and follows through an ATP depletion effect likely triggered by Ca++ ion tumor-cell entry, in addition to possible electro conformational denaturation of macromolecules, resulting from the EP probe pulsing (Aycock et al 2019; Novickij et al 2022). There are likely multiple mechanisms that drive IRE tumor cell damage. Curiously, the release of damage-associated molecular pattern (DAMP) molecules by EP correlates with tumor cell death (Polajzer et al 2020). Notably, the use of non-EP approaches such as alternating electric fields or external PMFs has resulted in nanopore formation (<20-nm pores) in tumor cells (Ashdown et al 2020; Chang et al 2018), which is associated with leak and membrane disruption (Chang et al 2018), while hydrophilic nanopore formation induced by EP approaches has been described (Aycock et al 2019). This may promote varying degrees of Ca++ entry and depending on extracellular Ca++, release of ATP and other DAMPs, lipid peroxidation, and downstream generation of ROS as well as induction of apoptotic pathways or more severe cellular injury via mechanisms as discussed (Novickij et al 2017; Aycock et al 2019; Novickij et al 2022). This further implies that tumor antigen release and immunologic priming may take place in such a microenvironment. The ability to potentially achieve the same membrane Ca++ inward-leak effect by applying coils and low-frequency pulses outside the body (or applied regionally across a body section) is intriguing. This is also in appreciation of the penetrative ability of PMFs, with deep-tissue induced EMFs and noting that studies with directly applied IRE pulses employ dB/dt typically in the microsecond (e.g., <100 μs) pulse-width range in several clinical applications (Ay).

Multiple modalities considered above may drive tumor sensitivity to EM waves, with a variety of stress or cytotoxic tumor-cell response mechanisms that may vary significantly depending on the modality. Indeed, one or two modalities may critically affect common tumor-responsive elements such as the glycocalyx, altered mitochondrial membrane properties, and ROS sensitivity (Xu et al 2021; Vadala et al 2016; Wang et al 2017; Novickij et al 2017; Blackstock et al 2000). Deciphering which physical parameter (e.g., EM wave amplitude or power, PMF pulse profile, dB/dt, and pulse frequency) may have the greatest impact on a specific tumor cell response will help in understanding how one can leverage a unique platform to achieve tumor membrane stress, apoptosis, cytotoxicity, or antigen release. The selectivity of “tuning” any empirically effective parameter to vulnerable tumor-specific cell or organelle biophysical characteristics may be an especially attractive feature while limiting strain or cytotoxicity to surrounding stromal or physiologic tissue. This is also in the spirit of the general paradigm presented herein, with a focus on preserving the function of tumor-associated or trafficking T cells, natural killer (NK) cells, and antigen-presenting cells (APCs). In some experimental applications that expose carcinoma cells and even neoplastic myeloid/lymphoid cells (leukemic and lymphoma lines) to EMFs, bystander non-neoplastic primary immune cells (including lymphocytes) under the same exposure remain more resilient or intact from apoptosis or DNA fragmentation (Hisamitsu et al 1997; Radeva et al 2004; Justesen et al 2022), while more generally in EMF exposed whole neoplasms, the activation and expansion of anti-tumor immune effector cells appears to be the rule (Justesen et al 2022).

Focusing on immune cells as the latter substituents to preserve and empower, an EM platform delivering the appropriate “dominant” parameter discovered from prior testing (e.g., tumor ROS generation under a unique low-frequency PMF pulse train with micro- or nanosecond pulse widths) may promote the anti-tumor function(s) of such cells without destroying them. This may ideally promote tumor antigen release and cross-presentation in a cytotoxic T cell-rich environment. Achieving this may spare immune cells and regional lymphatics participating in primary anti-tumor immunization from excessive stress or death that ensues from parallel chemotherapy or ionizing radiotherapy approaches that have traditionally been seen as “ideal” to potentiate a tumor vaccine response or an augmented cytotoxic T-cell response using antibodies to key co-inhibitory targets (e.g., anti-PD1 or anti-CTLA4) (Saddawi-Konefka et al 2022; Zitvogel et al 2008; Sharma et al 2024). One may thus envision preservation or promotion of the natural substrates for an optimal anti-tumor cytotoxic response with greater anti-tumor “signal-1” (tumor antigen presentation) potential as a result of the novel tumor-strain/apoptotic and tumor-cytotoxic conditions induced by the EM platform.

Electromagnetic induction has the potential to “loosen” barriers and engage levers to augment acquired T-cell immunity and checkpoint drivers while avoiding the ablation of effector immune cells in the same environment. Chemotherapy and ionizing radiation have the detrimental potential to eliminate or impair functional anti-tumor immune cells in the TME, including immunologic cells that mediate regional anti-tumor immunity (Zitvogel et al 2008; Sharma et al 2024). These may include naïve T cells, innate immune cells, or cells that have acquired specific immunity, including CD8+ cytotoxic T cells as well as CD4+ effectors sensitized to tumor antigens. It is intriguing to consider how TME immune barriers may be overcome or eliminated under unique EM platforms:

EM engagement with physical properties of the tumor glycocalyx. With unique upregulation of anionic sulfated glycans such as Sia or charged polymer (e.g., sulfated GAG or hyaluronan) modifications that project into the tumor glycocalyx in a variety of carcinomas, the potential to induce tumor membrane shear stress through pulsed EMF engagement with a “slab” of glycocalyx exists. In a macroscopic sense, a slab of glycocalyx covering a continuous layer of multiple cancer cells may potentially be driven into pulsed motion, creating shear on the tumor membrane via strain on protein and lipid attachments of the glycocalyx to the membrane (Johns et al 2023). This can induce membrane leak and downstream cell stress (Ashdown et al 2020; Johns et al 2023). This must be examined methodically for distinct tumors, and if PMFs are used, sufficient dB/dt to induce capture and force (slab mass×acceleration) may be achieved, rather than frequency or even amplitude of the field per se. Moreover, if this affects tumor glycocalyx with relative selectivity, without engaging glycan surfaces of stromal or immune cells (with markedly lower surface anionic charge densities), then this differential may allow optimum conditions for EM platform-induced (and relatively tumor-selective) apoptosis, antigen release, and sensing by TME immune cells.

EM induction of molecular force and torque: Consequences on tumor cell strain and viability. In tumor cells rich in proteins and lipids modified heavily by charged glycans, forces by pulsed B-field induced EMFs may induce motion of heavily charged glycosylated “head” regions of molecules, with a significant lever arm about the base of molecular attachment to the plasma membrane. This torque has the potential to deform the membrane with forces in the pico-Newton range (Nickels et al 2016; Falleroni et al 2018). Glycosidic bonds are generally strong and potentially less likely to “give”, thus transmitting forces into marked deformation and strain at the membrane-attached bases of proteoglycan core proteins, glycoproteins, or glycolipids; see references for examples of orders of magnitude pertaining to relative free energies of dissociation (Tsioptsias et al 2023; Sorensen et al 2015; Corey et al 2019). This may result in either a leak near points of torque (i.e., “weak points”) or mechanical transduction of neighboring channel proteins to affect ionic or small-molecule transport (Hart et al 2008). Empirically, the effects have also been shown to induce outward leak of plasma proteins, including proteases as well as the formation of nanopores with diameters in the 5-20-nm range: when electromagnetic pulse widths are substantially under the μs range, pore sizes appear to become substantially smaller (Ashdown et al 2020; Chang et al 2018; Son et al 2014).

Stress, leak, and cell death by any electroporation modality in tumor cells. Ideally, one can achieve “selective electroporation” as a result of engaging a unique EM platform (e.g., PMF-induced EMFs) with tumor glycocalyx to ultimately drive leak and cell stress, for example, via tumor membrane nanopore formation. The same platforms may remain relatively inert in neighboring non-tumor cells as a result of distinct (non- or low-charged) glycocalyces and the absence of overexpressed Sia or GAGs (Chin-Hun Kuo et al 2018; Johns et al 2023; Hart et al 2010). Interestingly, the induction of aminocyanine modifications on tumor membranes followed by light exposure was sufficient to induce rapid pore formation via “molecular jackhammers” through vibronic action on tumor plasma membranes, and immediate cell death. This is a novel two-step method for the transduction of vis-light waves to dramatic cytolytic effects in tumor monolayers and mouse tumor models (Ayala-Orozco et al 2024). The effect is appealing for carcinoma applications and possibly novel clinical translation.

Release of tumor-antigen targets in an immune “desert”. Could the immediate or delayed effects of selective tumor-cell electroporation and/or downstream apoptosis as a result of EM-induced cancer cell stress result in the release of tumor (neo)antigens into the TME while leaving bystander naïve or sensitized anti-tumor T cells relatively unharmed? The destruction of immune cells, including T and B cells in the TME as well as secondary lymphoid organs (lymph nodes) or TLS domains on nearby bronchovascular bundles, can be the unfortunate result of chemo- or ionizing radiotherapy, resulting in a loss in the opportunity for sensing of tumor antigen(s) as a “side effect” of classical tumor ablative modalities (Saddawi-Konefka et al 2022; Zitvogel et al 2008; Sharma et al 2024). This may create an immunologic “desert” within heterogeneous TME regions, with tumor-antigen targets as a lost opportunity in a setting of immunologic cell death or stress. Alternatively, immunity maybe harnessed broadly in a modality that is potentially more selective in inducing tumor-specific stress, including apoptosis/necroptosis while “sparing” immunologic viability in a setting of EM field exposure.

The carcinoma ECM is also rich in charged glycans and primed for mobilizing immunity. It maybe envisioned that engagement of EM platforms with charged molecular components of a tumor glycocalyx (e.g., glycocalyx-resonant EM waves or PMFs inducing EMFs that drive tumor molecular torque) may also engage susceptible poly-anionic matrix molecules that may not be tightly attached to tumor plasma membranes. Rather, secreted GAG or Sia decorated mucin-rich tumor cell products surrounding tumor nests or modifying basement membranes as glycan-rich ECM barriers may be vulnerable to EM waves or magnetic pulsing to disrupt or “loosen” a kinetic barrier to immunologic traffic (Wang et al 2022; Fujimori et al 2021), including both tumor antigen sensitized or naïve T cells, gaining access to tumor-cell products, which also include apoptotic or tumor dead-cell fragments. Reductions in matrix heparan sulfate and decorin have also been noted following the IRE of intact lungs in animal models (Fujimori et al 2021). This may potentially remodel the peri-tumor ECM in a way that improves anatomic points of entry for effector immune cells, but one can also consider whether this could change the “exit potential” for tumor cells from nests, considering potential consequences on local invasion. This maybe empirically weighed against a greater exposure substrate for anti-tumor immunity that may be driven by this potential interplay. One can question how this may be visualized or measured. When surviving sensitized T cells are physically distanced by significant interstitium or ECM, then EM field effects on matrix itself can facilitate T-cell penetration or narrow the bridge length remaining for CTLs to make signal 1 (MHC/antigen) contacts on tumor surfaces. In this light, EM exposures may also facilitate bi-specific T-cell engager (BiTE) compounds tarlatamab (Tang et al 2023), which engage a bridge between CD3 on the T cell and DLL3 on small cell lung carcinoma cells.

Promoting transfer of immune-cellular or anti-tumor therapeutics to glycan “vulnerable” cancer cells. The focus here is promoting greater “proximity” between potential/naïve or sensitized immune effector cells [e.g., dendritic cells (DCs), T cells, and NK cells] and/or therapeutic agents and tumor nests. Tumor vascular proliferation, primarily stimulated under common VEGF-A splice variants (e.g., VEGF-165) in tumor endothelium, is not only tortuous but also leaky (hence original term vascular permeability factor/VPF for VEGF-A) (Patel et al 2023). It promotes a net effect of increasing intra-tumor interstitial pressure, which further limits the ability of systemic or tumor-peripheral immune cells to gain access to viable tumors while accelerating necrosis in other parts of the tumor (Jain et al 2014). The latter may contain some antigen for sensing, but the absence of viable tumors (and immune cells) in such regions may hamper quality immune responses or outward traffic of newly sensitized T cells from the toxic environment (Harkos et al 2024). Interestingly, there are reports of pulsed EM radiofrequency fields generated on patient- or petoriented rings (e.g., Assisi or Beamer coils) used in anti-inflammatory and wound-healing contexts in veterinary practice for promoting improved or “normalized” blood flow (e.g., in granulation tissue) to facilitate wound recovery or improved inflammatory healing rates (e.g., post-traumatic musculoskeletal injury) in large animals (Vadala et al 20161 Gaynor et al 2018). Whether this “straightens” or normalizes vasculature in tumors to create a similar response in tumor blood flow as that of anti-VEGF therapy (Cesca et al 2016), following the discovery of VEGF-A induced “tortuous” tumor angiogenesis, and improves/lowers tumor interstitial pressure is unclear. Nevertheless, the concept of “normalizing” tumor vasculature through EM platform exposure while promoting anti-tumor immunity remains appealing as a strategy to promote a functional and “penetrant” tumor vasculature as a result of unique remodeling (via unknown mechanisms) while reducing tumor interstitial pressure.

Promoting lymphatic recruitment of secondary and tertiary lymphoid centers. It is appealing to envision that tumor antigens that can activate germinal centers in secondary lymphoid organs could flow (as metastatic or dying tumor cells or even free antigens) to regional lymph nodes via draining lymphatic vessels. These include secondary or even tertiary lymphoid structures (along lung bronchovasculature, for example), providing antigen substrate for APC cross-presentation with immune activation and proliferation of anti-tumor cytotoxic T cells in the lymphoid organ(s) (Preet Kaur et al 2023). Of course, the same conduit can carry live tumor cells with the danger of lymph node metastasis. Activated DCs (as master APCs) can also traffic in the same lymphatic beds, carrying processed tumor antigens, with downstream presentation events in draining lymph nodes. This may further include tertiary lymphoid structures (e.g., TLS domains along peri-tumor and lobar bronchovascular bundles in the setting of lung cancer). In any EM platform that can potentially impact/lessen tumor interstitial pressure through vascular remodeling or “normalization”, it is possible that such lymphatic traffic can drain even deeper portions of the tumor while potentially boosting antigen release into the draining “pool” in the form of greater apoptotic tumor cells or free antigen-rich molecular products following pulsing or electroporation, for example (Justesen et al 2022).

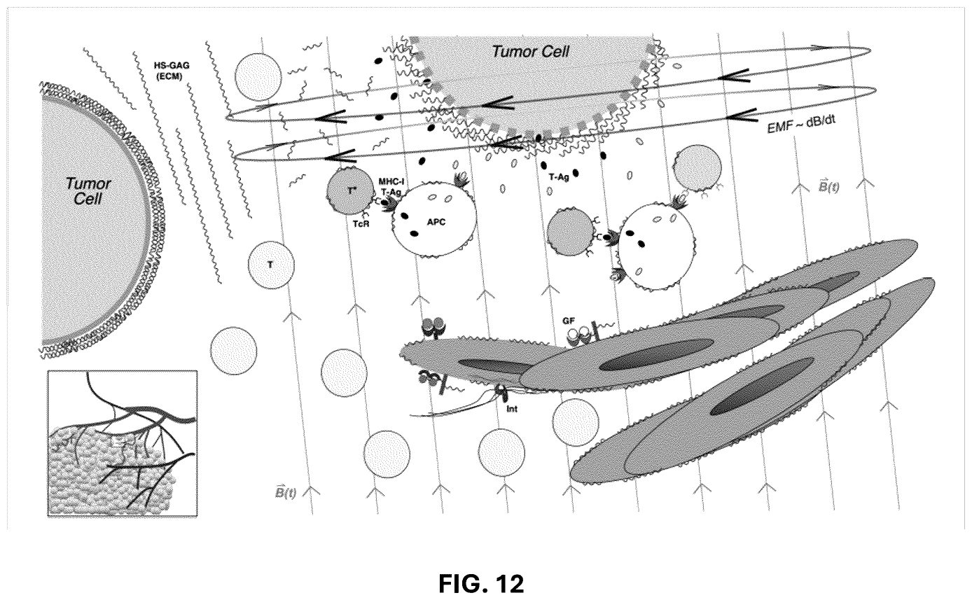

FIG. 12 illustrates how an EM stimulus that engages unique properties of the tumor plasma membrane (and even ECM) may promote mechanisms resulting in ant-tumor immune activation. Some of the above “substrates” for acquired immunologic activation and expansion are shown in the stromal and vascular microenvironment of the tumor, with the cartoon highlighting examples of glycocalyx sensitivity and engagement, possibly inert or physiologic molecular players, and secondary steps that promote immune activation against a stressed tumor cell within the penumbra of an EM induction field.

Table 1 summarizes a variety of cellular mechanisms by which EM platforms may augment anti-tumor immunity via induction in a non-ionizing and non-thermal manner. Some of these may occur through indirect effects of tumor cell strain or death in the microenvironment of naïve and/or sensitized T cells, antigen-presenting cells, NK cells, and vascular endothelial cells, among others.

TABLE 1
Mechanisms by which non-ionizing, non-thermal
EM platforms may augment anti-tumor immunity

| Function | Effector cells impacted | Possible mechanism(s) |
|---|---|---|
| CD8+ T-cell | Naïve and effector T | Release of antigen from EM-driven tumor |
| antigen priming | cells | apoptosis or necrosis. Nanopore formation |
| | | by reversible or irreversible electroporation |
| Tumor antigen | APCs (mainly cDC1 | Augmented antigen cross-presentation in |
| sensing | DCs) | setting of unstable (pule-vibrated) Sia or |
| | | GAG chains (intact glycans regulate |
| | | presentation spatially/temporally) |
| “Open” or | APCs and T cells | EM induced inhibition in matrix density |
| decompress ECM | (naïve and effector) | (temporal), facilitated immunological cell |
| for effector | | penetration to tumor mass |
| migration/kinetics | | |
| Inhibit glycan- | NK cells, T cells. Also | Perturbation or downregulation in Sia |
| mediated immune | DCs “in trans” (matrix) | (considered a “resistance” response) on |
| repression | or “in cis” | immune cells may inhibit Siglec-mediated |
| | | immunosuppressive signaling |
| Possible deep | Endothelial cells in the | Vascular remodeling; possible |
| regional tumor | TME; circulating T | “normalizing” effects on tumor vessels, |
| immune-cell | cells | reducing tumor interstitial pressure |
| access | | |

### DISCUSSION

The data disclosed herein shows the integration of an EM carcinoma cell stress modality with immunologic sensing and tumor-cell contact by well-primed T cells. This includes contact with non-viable tumor cells and/or antigen products as debris evolving from EM-induced apoptotic and necroptotic tumor cells in the TME. In the setting of EM wave exposures induced either “at a distance” by external PMF induction or penetrating low-frequency radiofrequency EM waves or by a tumor-proximal probe source (e.g., irreversible electroporation source deployed to the surface of the tumor), the combination of tumor cell stress and antigen release is carried out while aiming to leave immunologic cell bystanders relatively intact and ready to prime in response to changes in the microenvironment (Justesen et al 2022; Aaes et al 2016). Emerging studies in animal models are demonstrating anti-tumor immunologic preservation (and boost) of non-thermal electroporative strategies, for example, in direct comparison to that of radiofrequency thermal ablative approaches in the same model (Nafie et al 2023). If cell-surface properties of DCs, T cells, NK cells, and stromal cells are unique and remain non-vulnerable to detrimental effects of EM stimuli (e.g., transduced by discrete or continuous glycocalyces of tumor cells), then one may achieve scenarios where tumor nests are stressed, even to a lethal extent that promotes tumor antigen release via apoptosis and necroptosis (Mercadal et al 2020; Peng et al 2024), while minimizing stress on the immune effectors. The latter has been shown to expand in the post-treatment TME of a variety of IRE-treated tumors in animals and humans (Zhang et al 2021).

The “sparing” and effective activation of effector T cells that may be achieved under EM platform-driven tumor-stress modalities in the TME may not be the case in most common chemotherapy or ionizing radiotherapy platforms, even when paired with immunotherapy. Further, while common current immunotherapy strategies for carcinoma are immune checkpoint inhibitor (ICI) based and operate optimally (and without antigen specificity) in response to target antigen/MHC-T-cell “signal one” events, exposure to tumor-stress focused EM platforms may augment signal one response by bystander naïve T cells (or antigen-driven expansion of tumor-sensitized T cells) in the same TME in real time. This may facilitate exposure of tumor-generated immune substrates for acquired anti-tumor immunity and memory T-cell generation despite a constantly evolving carcinoma antigen landscape. A challenge in the evolution of tumor surface “contact” PEF platforms in particular is maintaining an even exposure of any PEF over the full tumor landscape due to the electric heterogeneity of tumor tissue (Wang et al 2019), leaving the potential for tumor recurrence in differentially exposed regions with incomplete IRE-directed ablation (Zhang et al 2021). Possibly, the use of locally delivered PMFs may expose the full thickness of tumors to induced EMFs (dB/dt) more evenly due to uniform B-field penetration and thus more uniform release of the variety of antigens in the evolving heterogeneous tumor landscape.

As an example, in lung cancer as well as metastatic tumors to the lung, the use of bronchoscopic IRE is being investigated for augmentation of the unique effects that this EM platform has on inducing a local effector tumor immunophenotype, characterized by increased DCs, effector T cells, inhibition in T-suppressor (Treg subset) cells, and augmented regional TLS activation, while IRE has been more generally shown to spare stromal and vascular cells within the TME (Wang et al 2022; Zhang et al 2021; Jimenez et al 2023). This specific PEF platform, which is growing in very early clinical application, delivers electric pulses directly to the center of the lung tumor with the bronchoscope-deployed IRE probe, wherein energy is the greatest in a central IRE ablation zone, while a gradient likely exists wherein tumor immune cells are differentially sensitized to the radially outward attenuating E-field delivered with sufficient energy to induce tumor-cell stress and/or death without appreciable thermal effects on surrounding vascular/stromal tissues (Wang et al 2022; Jimenez et al 2023). The tumor cell stress and release of antigens in this context may boost cross-presentation to CD8+ T cells with the induction of cytolytic effector cells indistinct regions. One could conjecture that regional intra-tumor or cross-tumor delivery of a PMF in such a “probe-delivered” manner could also non-thermally and uniformly alter distinct clonal tumor populations through the full tumor thickness to release “regional” antigens across the heterogeneous carcinoma. Boosting this with ICI or other novel approaches while the local immune system is preserved may provide a unique variation on this theme.

For at least a large subset of tumors expressing heavy membrane Sia modifications (e.g., as poly-sialic acid or Sia-modified glycoproteins and glycolipids/gangliosides) on the tumor-cell glycocalyx (Chin-Hun Kuo et al 2018; Pinho et al 2015; Miyahara et al 2001; Suzuki et al 2005), a natural “resistance” response to escape EM-induced tumor stress may evolve through the tumor's attempt to reduce Sia expression, thus reducing the tumor's dense glycan anionic charge layer. However, a high density of tumor glycocalyx Sia (particularly polysialic acid polymers) has been shown to be important for physical tumor cell-cell repulsion at a tissue-invading tumor front, including repulsion from immune effector cells (Fuster et al 2005; Suzuki et al 2005; Drake et al 2008). It may also mediate binding of chemokines and cytokines that affect tumor cell migration and behavior (Drake et al 2008). Heavy glycocalyx Sia also induces immunologic shielding from anti-tumor immune cells through the activation of repressive Sia-binding Siglecs on immune-cell surfaces (for example, NK-surface Siglec 7-, 9-, or NKG2D-mediated repression of NK cells by engagement with heavy Sia on tumor glycocalyx) (Zheng et al 2020; Hudak et al 2014). Siglec-mediated repression by tumor-surface Sia expression is also a theme for other anti-tumor immune cells in the TME. In yet another scenario of potential EM-platform tumor resistance, remodeling attempts by tumor cells to reduce the expression of anionic GAGs such as hyaluronan or sulfated HS chains (which can “dominate” the glycocalyx mass in a variety of carcinomas) to escape or survive EM-induced tumor-cell stress may lead to tumor growth inhibition due to loss of major growth factor co-receptor functions by such GAGs (Fuster et al 2005; Vlodavsky et al 2023; Kang et al 2018). A reduction in “banks” of growth factors bound to secreted GAGs in the tumor ECM in this scenario may also result in reduced tumor invasion through basement membranes and tissue barriers (Vlodavsky et al 2023). Therefore, the loss of repulsion, immune escape, or growth potential resulting from tumor attempts to reduce anionic tumor glycocalyx constituents as an EM-platform resistance mechanism may result in a “checkmate” situation for the glycan-addicted tumor cell that must hover between EM-platform vulnerability and loss of invasive and immune-shielding potential.

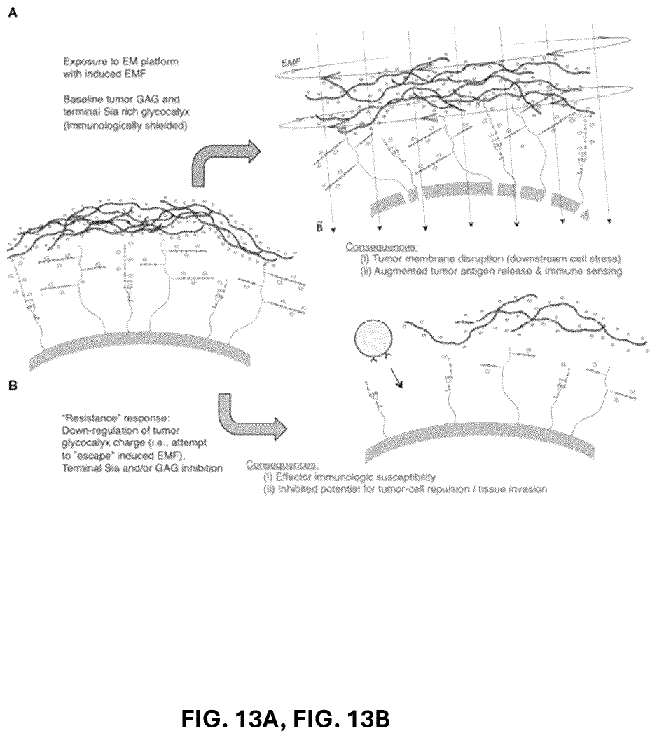

FIG. 13 illustrates scenarios of i) EM platform exposure that disrupts the tumor membrane due to glycocalyx “slab” movement and molecular torque that may occur on an EM-strained and disrupted tumor membrane, inducing leak and downstream mechanisms that promote tumor cell stress and potentially tumor cell lethality (as a mechanism for augmented tumor antigen exposure in the TME). ii) Under temporal “resistance” by an exposed tumor cell, escape from these effects is possible, but with consequences of heightened immunologic susceptibility due to the downregulation of glycans that typically serve to repress incoming anti-tumor NK cells or T cells through a variety of glycan-receptor (e.g., Siglec and other) immunologic cell-inhibitory pathways.

Another step disclosed is the pairing of a form of immunotherapy (even current-day ICI strategies) with an EM wave platform that can be specifically “tuned” for optimal tumor membrane stress from a fresh biopsy specimen from the tumor with its accompanying stroma. Effectively, with any real-time tumor specimen, one could determine a ratio of cytotoxic “leak” of proteases (or inward leak of a tracking molecule such as propidium iodide) by tumor cells versus that of stromal cells separated from the same specimen (Johns et al 2023). In classical dynamic scenarios of mechanical resonance (Marion et al 2013), this is essentially the resonance “quality factor” of the tumor-glycocalyx (as an EM-wave susceptible system) relative to that of non-tumor cells in the immediate environment.

### Example 5

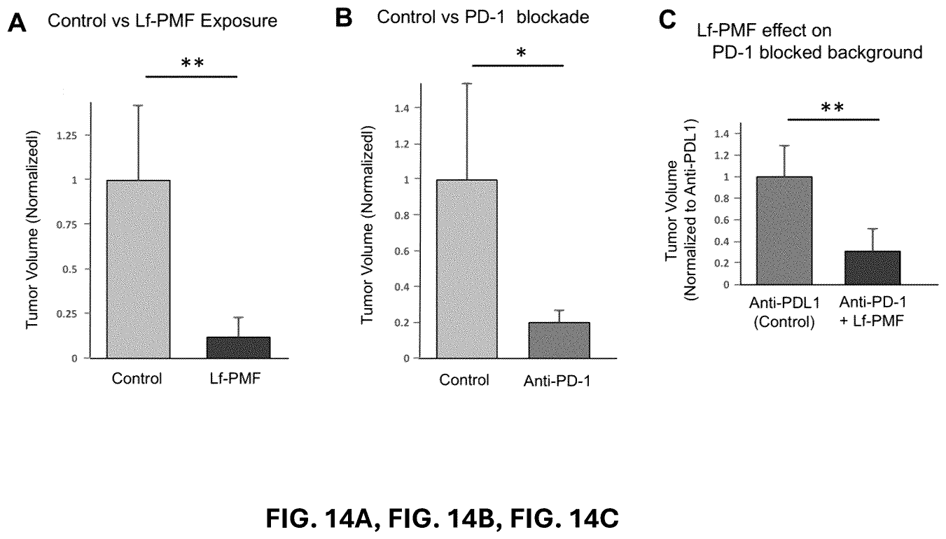

Lewis lung carcinoma (LLC) is a syngeneic mouse lung tumor model that is generally modestly responsive to blocking the PDL1-PD1 immune checkpoint inhibition axis, and otherwise useful for testing anti-tumor therapies in immunocompetent mice. This data demonstrates that a therapy can potentiate modest anti-tumor immune checkpoint effects in this model may suggest that the novel therapy may at least potentiate Signal 1 (MHC-I/Antigen—CD8+ T cell receptor interaction) of the immune synapse. While mechanistic studies are ongoing, further studies on the basic finding that a daily 15 min exposure to an extremely low frequency pulsed magnetic field (PMF) delivered in 15 Hz pulse trains using relatively sharp pulsing (˜70 msec duty cycle) with a modified pulse-coil (MagVenture cool-40 rat coil) overlying a growing LLC subcutaneous tumor over 5 days results in marked tumor inhibition relative to non-exposed sham treated controls (FIG. 14A). In this model, to demonstrate immunotherapy responsiveness, antibody inhibition of the PDL1-PD1 immune checkpoint axis results in significant inhibition in tumor growth (FIG. 14B). Knowing that this anti-tumor immune sensitivity exists in the model, to assess the effects of low frequency PMF exposure on a background of ongoing immune checkpoint (T-cell PD-1 receptor) blockade, PMF exposures were applied to a subset of anti-PD-1 antibody treated mice, noting that the combined treatment moderately and significantly inhibited tumor growth over that of the anti-PD-1 control state (FIG. 14C, right).

This shows the in vivo capacity of low frequency PMFs to not only markedly inhibit LLC tumor growth, but also the ability to potentiate immune-inhibitory effects of immune checkpoint blockade in the system. Direct effects on tumor cells via glycocalyx mediated cancer cell-stress and apoptosis/death may also be contributory (which also potentiates the immunologic/antigen-accessibility effect within the tumor).

Those skilled in the art will recognize, or be able to ascertain using no more than routine experimentation, many equivalents to the specific embodiments of the method and compositions described herein. Such equivalents are intended to be encompassed by the following claims.

### REFERENCES

Aaes T L, Kaczmarek A, Delvaeye T, De Craene B, De Koker S, Heyndrickx L, et al. Vaccination with necroptotic cancer cells induces efficient anti-tumor immunity. Cell Rep. (2016) 15:274-87. doi: 10.1016/j.celrep.2016.03.037
Alekseeva A, Urso E, Mazzini G, Naggi A: Heparanase as an Additional Tool for Detecting Structural Peculiarities of Heparin Oligosaccharides. Molecules (Basel, Switzerland) 2019, 24(23).
Amoureux M C, Coulibaly B, Chinot O, Loundou A, Metellus P, Rougon G, Figarella-Branger D: Polysialic acid neural cell adhesion molecule (PSA-NCAM) is an adverse prognosis factor in glioblastoma, and regulates olig2 expression in glioma cell lines. BMC Cancer 2010, 10:91.
Arango-Restrepo A, Rubi J M. Predicting cancer stages from tissue energy dissipation. Sci Rep. (2023) 13:15894. doi: 10.1038/s41598-023-42780-0
Ashdown, C. P., S. C. Johns, E. Aminov, M. Unanian, W. Connacher, J. Friend, and M. M. Fuster. 2020. Pulsed Low-Frequency Magnetic Fields Induce Tumor Membrane Disruption and Altered Cell Viability. Biophysical journal 118(7):1552-1563.
Ayala-Orozco C, Galvez-Aranda D, Corona A, Seminario J M, Rangel R, Myers I N, et al. Molecular jackhammers eradicate cancer cells by vibronic-driven action. Nat Chem. (2024) 16:456-65. doi: 10.1038/s41557-023-01383-y
Aycock K N, Davalos R V. Irreversible electroporation: background, theory, and review of recent developments in clinical oncology. Bioelectricity. (2019) 1:214-34. doi: 10.1089/bioe.2019.0029
Barbault A, Costa F P, Bottger B, Munden R F, Bomholt F, Kuster N, et al. Amplitude-modulated electromagnetic fields for the treatment of cancer: discovery of tumor-specific frequencies and assessment of a novel therapeutic approach. J Exp Clin Cancer Res C R. (2009) 28:51. doi: 10.1186/1756-9966-28-51
Bartik, P., A. Maglott, G. Entlicher, D. Vestweber, K. Takeda, S. Martin, and M. Dontenwill. 2008. Detection of a hypersialylated beta1 integrin endogenously expressed in the human astrocytoma cell line A172. International journal of oncology 32(5):1021-1031.
Batista Napotnik T, Polajžer T, Miklavčič D. Cell death due to electroporation—A review. Bioelectrochem (Amsterdam Netherlands). (2021) 141:107871. doi: 10.1016/j.bioelechem.2021.107871
Bellis S L, Reis C A, Varki A, Kannagi R, Stanley P. Glycosylation changes in cancer. In: Varki A, Cummings R D, Esko J D, Stanley P, Hart G W, Aebi M, Mohnen D, Kinoshita T, Packer N H, Prestegard J H, Schnaar R L, Seeberger P H, editors. Essentials of Glycobiology. Cold Spring Harbor Laboratory Press Copyright © 2022 The Consortium of Glycobiology Editors, La Jolla, California; published by Cold Spring Harbor Laboratory Press, Cold Spring Harbor (NY: (2022). p. 631-44.
Bergandi L, Lucia U, Grisolia G, Salaroglio I C, Gesmundo I, Granata R, et al. Thermomagnetic resonance effect of the extremely low frequency electromagnetic field on three-dimensional cancer models. Int J Mol Sci. (2022) 23:1-19. doi: 10.3390/ijms23147955
Berry L, Andrew M, Post M, Ofosu F, O'Brodovich H: A549 lung epithelial cells synthesize anticoagulant molecules on the cell surface and matrix and in conditioned media. Am J Respir Cell Mol Biol 1991, 4(4):338-346.
Bhattacharya A, Mrudula K, Sreepada S S, Sathyaprabha T N, Pal P K, Chen R, et al. An Overview of Noninvasive Brain Stimulation: Basic Principles and Clinical Applications. The Canadian journal of neurological sciences. Le J Canadien Des Sci Neurologiques. (2022) 49:479-92. doi: 10.1017/cjn.2021.158
Blackstock D T. Fundamentals of Physical Acoustics. Wiley, New York, NY: (2000).
Bojar, D., L. Meche, G. Meng, W. Eng, D. F. Smith, R. D. Cummings, and L. K. Mahal. 2022. A Useful Guide to Lectin Binding: Machine-Learning Directed Annotation of 57 Unique Lectin Specificities. ACS chemical biology 17(11):2993-3012.
Buffone, A., and V. M. Weaver. 2020. Don't sugarcoat it: How glycocalyx composition influences cancer progression. The Journal of cell biology 219(1).
Carrasco-Padilla C, Hernaiz-Esteban A, Alvarez-Vallina L, Aguilar-Sopeña O, Roda-Navarro P. Bispecific antibody format and the organization of immunological synapses in T cell-redirecting strategies for cancer immunotherapy. Pharmaceutics. (2022) 15:1-14. doi: 10.3390/pharmaceutics15010132
Cesca M, Morosi L, Berndt A, Fuso Nerini I, Frapolli R, Richter P, et al. Bevacizumab-induced inhibition of angiogenesis promotes a more homogeneous intratumoral distribution of paclitaxel, improving the antitumor response. Mol Cancer Ther. (2016) 15:125-35. doi: 10.1158/1535-7163.MCT-15-0063
Chang, E., C. B. Patel, C. Pohling, C. Young, J. Song, T. A. Flores, Y. Zeng, L. M. Joubert, H. Arami, A. Natarajan, R. Sinclair, and S. S. Gambhir. 2018. Tumor treating fields increases membrane permeability in glioblastoma cells. Cell death discovery 4:113.
Chen, Y., Z. Fan, Y. Yang, and C. Gu. 2019. Iron metabolism and its contribution to cancer (Review). International journal of oncology 54(4):1143-1154.
Chin-Hun Kuo J, Gandhi J G, Zia R N, Paszek M J: Physical biology of the cancer cell glycocalyx. Nature physics 2018, 14(7):658-669.
Corey R A, Vickery O N, Sansom M S P, Stansfeld P J. Insights into membrane protein-lipid interactions from free energy calculations. J Chem Theory Comput. (2019) 15:5727-36. doi: 10.1021/acs.jctc.9b00548
Crowley L C, Marfell B J, Scott A P, Waterhouse N J: Quantitation of Apoptosis and Necrosis by Annexin V Binding, Propidium Iodide Uptake, and Flow Cytometry. Cold Spring Harbor protocols 2016, 2016(11).
Cuello, H. A., G. M. Ferreira, C. A. Gulino, A. G. Toledo, V. I. Segatori, and M. R. Gabri. 2020. Terminally sialylated and fucosylated complex N-glycans are involved in the malignant behavior of high-grade glioma. Oncotarget 11(52):4822-4835.
Dae, H. M., H. Y. Kwon, N. Y. Kang, N. R. Song, K. S. Kim, C. H. Kim, J. H. Lee, and Y. C. Lee. 2009. Isolation and functional analysis of the human glioblastoma-specific promoter region of the human GD3 synthase (hST8Sia I) gene. Acta biochimica et biophysica Sinica 41(3):237-245.
Dai C H, Li J, Yu L C, Li X Q, Shi S B, Wu J R: Molecular diagnosis and prognostic significance of lymph node micrometastasis in patients with histologically node-negative non-small cell lung cancer. Tumour biology: the journal of the International Society for Oncodevelopmental Biology and Medicine 2013, 34(2):1245-1253.
Dandona, P., T. Qutob, W. Hamouda, F. Bakri, A. Aljada, and Y. Kumbkarni. 1999. Heparin inhibits reactive oxygen species generation by polymorphonuclear and mononuclear leucocytes. Thrombosis research 96(6):437-443.

de Seze, R., S. Tuffet, J. M. Moreau, and B. Veyret. 2000. Effects of 100 mT time varying magnetic fields on the growth of tumors in mice. Bioelectromagnetics 21(2):107-111.
Drakaki M, Mathiesen C, Siebner H R, Madsen K, Thielscher A: Database of 25 validated coil models for electric field simulations for TMS. Brain Stimul 2022, 15(3):697-706.
Drake P M, Nathan J K, Stock C M, Chang P V, Muench M O, Nakata D, et al. Polysialic acid, a glycan with highly restricted expression, is found on human and murine leukocytes and modulates immune responses. J Immunol (Baltimore Md 1950). (2008) 181:6850-8. doi: 10.4049/jimmunol.181.10.6850
Falconer, R. A., R. J. Errington, S. D. Shnyder, P. J. Smith, and L. H. Patterson. 2012. Polysialyltransferase: a new target in metastatic cancer. Current cancer drug targets 12(8):925-939.
Falleroni F, Torre V, Cojoc D. Cell mechanotransduction with piconewton forces applied by optical tweezers. Front Cell Neurosci. (2018) 12:130. doi: 10.3389/fncel.2018.00130
Fujimori M, Kimura Y, Ueshima E, Dupuy D E, Adusumilli P S, Solomon S B, et al. Lung ablation with irreversible electroporation promotes immune cell infiltration by sparing extracellular matrix proteins and vasculature: implications for immunotherapy. Bioelectricity. (2021) 3:204-14. doi: 10.1089/bioe.2021.0014
Fuster, M. M., and J. D. Esko. 2005. The sweet and sour of cancer: glycans as novel therapeutic targets. Nature reviews. Cancer 5(7):526-542.
Fuster, M. M., L. Wang, J. Castagnola, L. Sikora, K. Reddi, P. H. Lee, K. A. Radek, M. Schuksz, J. R. Bishop, R. L. Gallo, P. Sriramarao, and J. D. Esko. 2007. Genetic alteration of endothelial heparan sulfate selectively inhibits tumor angiogenesis. The Journal of cell biology 177(3):539-549.
Fuster M M. Integrating electromagnetic cancer stress with immunotherapy: a therapeutic paradigm. Front Oncol. 2024; 14:1417621. Published 2024 Aug. 6. doi:10.3389/fonc.2024.1417621
Garcia P A, Kos B, Rossmeisl J H, Jr., Pavliha D, Miklavčič D, Davalos R V. Predictive therapeutic planning for irreversible electroporation treatment of spontaneous Malignant glioma. Med Phys. (2017) 44:4968-80. doi: 10.1002/mp.12401
Gaynor J S, Hagberg S, Gurfein B T. Veterinary applications of pulsed electromagnetic field therapy. Res Veterinary Sci. (2018) 119:1-8. doi: 10.1016/j.rvsc.2018.05.005
Gong L, Zhou X, Yang J, Jiang Y, Yang H: Effects of the regulation of polysialyltransferase ST8SiaII on the invasiveness and metastasis of small cell lung cancer cells. Oncology reports 2017, 37(1):131-138.
Gospodinov, A., and Z. Herceg. 2013. Chromatin structure in double strand break repair. DNA repair 12(10):800-810.
Gupta, P., S. C. Johns, S. Y. Kim, R. El Ghazal, E. I. Zuniga, and M. M. Fuster. 2020. Functional Cellular Anti-Tumor Mechanisms are Augmented by Genetic Proteoglycan Targeting. Neoplasia (New York, N.Y.) 22(2):86-97.
Hambarde S, Manalo J M, Baskin D S, Sharpe M A, Helekar S A. Spinning magnetic field patterns that cause oncolysis by oxidative stress in glioma cells. Sci Rep. (2023) 13:19264. doi: 10.1038/s41598-023-46758-w
Harkos C, Stylianopoulos T. Investigating the synergistic effects of immunotherapy and normalization treatment in modulating tumor microenvironment and enhancing treatment efficacy. J Theor Biol. (2024) 583:111768. doi: 10.1016/j.jtbi.2024.111768
Hart F X. Cytoskeletal forces produced by extremely low-frequency electric fields acting on extracellular glycoproteins. Bioelectromagnetics. (2010) 31:77-84. doi: 10.1002/bem.20525
Hart F X. The mechanical transduction of physiological strength electric fields. Bioelectromagnetics. (2008) 29:447-55. doi: 10.1002/bem.20411
Head, B. P., H. H. Patel, and P. A. Insel. 2014. Interaction of membrane/lipid rafts with the cytoskeleton: impact on signaling and function: membrane/lipid rafts, mediators of cytoskeletal arrangement and cell signaling. Biochimica et biophysica acta 1838(2):532-545.
Hisamitsu T, Narita K, Kasahara T, Seto A, Yu Y, Asano K. Induction of apoptosis in human leukemic cells by magnetic fields. Japanese J Physiol. (1997) 47:307-10. doi: 10.2170/jjphysiol.47.307
Hore P J, Mouritsen H. The radical-pair mechanism of magnetoreception. Annu Rev Biophysics. (2016) 45:299-344. doi: 10.1146/annurev-biophys-032116-094545
Huang M, Li P, Chen F, Cai Z, Yang S, Zheng X, et al. Is extremely low frequency pulsed electromagnetic fields applicable to gliomas?A literature review of the underlying mechanisms and application of extremely low frequency pulsed electromagnetic fields. Cancer Med. (2023) 12:2187-98. doi: 10.1002/cam4.5112
Hudak J E, Canham S M, Bertozzi C R. Glycocalyx engineering reveals a Siglec-based mechanism for NK cell immunoevasion. Nat Chem Biol. (2014) 10:69-75. doi: 10.1038/nchembio.1388
Jain R K. Antiangiogenesis strategies revisited: from starving tumors to alleviating hypoxia. Cancer Cell. (2014) 26:605-22. doi: 10.1016/j.ccell.2014.10.006
Jarahian M, Marofi F, Maashi M S, Ghaebi M, Khezri A, Berger M R. Re-expression of poly/oligo-sialylated adhesion molecules on the surface of tumor cells disrupts their interaction with immune-effector cells and contributes to pathophysiological immune escape. Cancers. (2021) 13:1-47. doi: 10.3390/cancers13205203
Jimenez M, Garcia-Hierro J F, Aldeyturriaga J F, v. d. Heijden E H, Ng C, Lau R, et al. 697 Pulsed Electric Fields induces a stepwise activation of host anti-tumor immunity in patients with early-stage non-small cell lung cancer (NSCLC). J ImmunoTher Cancer. (2023) 11:A789-91. doi: 10.1136/jitc-2023-SITC2023.0697
Johns S C, Gupta P, Lee Y H, Friend J, Fuster M M. Glycocalyx transduces membrane leak in brain tumor cells exposed to sharp magnetic pulsing. Biophys J. (2023) 122:4425-39. doi: 10.1016/j.bpj.2023.10.020
Justesen T F, Orhan A, Raskov H, Nolsoe C, Gogenur I. Electroporation and immunotherapy-unleashing the abscopal effect. Cancers. (2022) 14:1-28. doi: 10.3390/cancers14122876
Kalli M, Stylianopoulos T. Toward innovative approaches for exploring the mechanically regulated tumor-immune microenvironment. APL Bioengineering. (2024) 8:011501. doi: 10.1063/5.0183302
Kang H, Wu Q, Sun A, Liu X, Fan Y, Deng X. Cancer cell glycocalyx and its significance in cancer progression. Int J Mol Sci. (2018) 19:1-23. doi: 10.3390/ijms19092484
Kielbik A, Szlasa W, Novickij V, Szewczyk A, Maciejewska M, Saczko J, et al. Effects of high-frequency nanosecond pulses on prostate cancer cells. Sci Rep. (2021) 11:15835. doi: 10.1038/s41598-021-95180-7
Kim, H., and X. Xue. 2020. Detection of Total Reactive Oxygen Species in Adherent Cells by 2′,7′-Dichlorodihydrofluorescein Diacetate Staining. Journal of visualized experiments: JoVE(160).
Koh, E. K., B. K. Ryu, D. Y. Jeong, I. S. Bang, M. H. Nam, and K. S. Chae. 2008. A 60-Hz sinusoidal magnetic field induces apoptosis of prostate cancer cells through reactive oxygen species. International journal of radiation biology 84(11):945-955.
Korshoej A R, Lukacova S, Lassen-Ramshad Y, Rahbek C, Severinsen K E, Guldberg T L, et al. OptimalTTF-1: Enhancing tumor treating fields therapy with skull remodeling surgery. A clinical phase I trial in adult recurrent glioblastoma. Neuro-oncol Adv. (2020) 2:vdaa121. doi: 10.1093/noajnl/vdaa121
Liu Z L, Chen H H, Zheng L L, Sun L P, Shi L. Angiogenic signaling pathways and anti-angiogenic therapy for cancer. Signal Transduction Targeted Ther. (2023) 8:198. doi: 10.1038/s41392-023-01460-1
Lo S L, Thike A A, Tan S Y, Lim T K, Tan I B, Choo S P, Tan P H, Bay B H, Yip G W: Expression of heparan sulfate in gastric carcinoma and its correlation with clinicopathological features and patient survival. Journal of clinical pathology 2011, 64(2):153-158.
Lucia U, Grisolia G. Seebeck-peltier transition approach to oncogenesis. Appl Sci. (2020) 10:7166. doi: 10.3390/app10207166
Macauley M S, Paulson J C. Immunology: glyco-engineering ‘super-self’. Nat Chem Biol. (2014) 10:7-8. doi: 10.1038/nchembio.1415
   Marion J B. Classical Dynamics of Particles and Systems. Academic Press, New York, NY: (2013).
Mercadal B, Beitel-White N, Aycock K N, Castellvi Q, Davalos R V, Ivorra A. Dynamics of cell death after conventional IRE and H-FIRE treatments. Ann Biomed Eng. (2020) 48:1451-62. doi: 10.1007/s10439-020-02462-8
Merry C L R, Lindahl U, Couchman J, Esko J D. Proteoglycans and sulfated glycosaminoglycans. In: Varki A, Cummings R D, Esko J D, Stanley P, Hart G W, Aebi M, Mohnen D, Kinoshita T, Packer N H, Prestegard J H, Schnaar R L, Seeberger P H, editors. Essentials of Glycobiology. Cold Spring Harbor Laboratory Press Copyright © 2022 The Consortium of Glycobiology Editors, La Jolla, California; published by Cold Spring Harbor Laboratory Press, Cold Spring Harbor (NY: (2022). p. 217-32.
Mills K R, Mills K R. Biophysics of magnetic stimulation. In: Magnetic Stimulation of the Human Nervous System. Oxford University Press, Northants, United Kingdom: (2000). p. 0.
Mitchell, M. J., and M. R. King. 2014. Physical biology in cancer. 3. The role of cell glycocalyx in vascular transport of circulating tumor cells. American journal of physiology. Cell physiology 306(2):C89-97.
Miyahara R, Tanaka F, Nakagawa T, Matsuoka K, Isii K, Wada H. Expression of neural cell adhesion molecules (polysialylated form of neural cell adhesion molecule and L1-cell adhesion molecule) on resected small cell lung cancer specimens: in relation to proliferation state. J Surg Oncol. (2001) 77:49-54. doi: 10.1002/jso.1065
Moser J C, Salvador E, Deniz K, Swanson K, Tuszynski J, Carlson K W, et al. The mechanisms of action of tumor treating fields. Cancer Res. (2022) 82:3650-8. doi: 10.1158/0008-5472.CAN-22-0887
Nafie E, Pastori C, Wagh M, Goldschmidt E, Neal R. 15 Pulsed electric field therapy increases immune response and improves murine 4T1 breast tumor local and metastatic outcomes relative to radiofrequency ablation. J ImmunoTher Cancer. (2023) 11:A15-7. doi: 10.1136/jitc-2023-SITC2023.0015
Nagarajan A, Malvi P, Wajapeyee N: Heparan Sulfate and Heparan Sulfate Proteoglycans in Cancer Initiation and Progression. Front Endocrinol (Lausanne) 2018, 9:483.
Nguyen V N, Mirejovsky T, Melinovi L, Mandys V: CD44 and its v6 spliced variant in lung carcinomas: relation to NCAM, CEA, EMA and UPi and prognostic significance. Neoplasma 2000, 47(6):400-408.
Nickels P C, Wunsch B, Holzmeister P, Bae W, Kneer L M, Grohmann D, et al. Molecular force spectroscopy with a DNA origami-based nanoscopic force clamp. Sci (New York N Y). (2016) 354:305-7. doi: 10.1126/science.aah5974
Nie, Y., L. Du, Y. Mou, Z. Xu, L. Weng, Y. Du, Y. Zhu, Y. Hou, and T. Wang. 2013. Effect of low frequency magnetic fields on melanoma: tumor inhibition and immune modulation. BMC cancer 13:582.
Nie, Y., Y. Chen, Y. Mou, L. Weng, Z. Xu, Y. Du, W. Wang, Y. Hou, and T. Wang. 2013. Low frequency magnetic fields enhance antitumor immune response against mouse H22 hepatocellular carcinoma. PloS one 8(11):e72411.
Novickij V, Dermol J, Grainys A, Kranjc M, Miklavčič D. Membrane permeabilization of mammalian cells using bursts of high magnetic field pulses. PeerJ. (2017) 5:e3267. doi: 10.7717/peerj.3267
Novickij, V., N. Rembiałkowska, P. Kasperkiewicz-Wasilewska, D. Baczyńska, A. Rzechonek, P. Błasiak, and J. Kulbacka. 2022. Pulsed electric fields with calcium ions stimulate oxidative alternations and lipid peroxidation in human non-small cell lung cancer. Biochimica et biophysica acta. Biomembranes 1864(12):184055.
Novikov V V, Novikov G V, Fesenko E E. Effect of weak combined static and extremely low-frequency alternating magnetic fields on tumor growth in mice inoculated with the Ehrlich ascites carcinoma. Bioelectromagnetics. (2009) 30:343-51. doi: 10.1002/bem.20487
Oetke, C., S. Hinderlich, R. Brossmer, W. Reutter, M. Pawlita, and O. T. Keppler. 2001. Evidence for efficient uptake and incorporation of sialic acid by eukaryotic cells. European journal of biochemistry 268(16):4553-4561.
Omote, Y., M. Hosokawa, M. Komatsumoto, T. Namieno, S. Nakajima, Y. Kubo, and H. Kobayashi. 1990. Treatment of experimental tumors with a combination of a pulsing magnetic field and an antitumor drug. Japanese journal of cancer research: Gann 81(9):956-961.
Pall M L. Millimeter (MM) wave and microwave frequency radiation produce deeply penetrating effects: the biology and the physics. Rev Environ Health. (2022) 37:247-58. doi: 10.1515/reveh-2020-0165
Parthoens, J., J. Verhaeghe, S. Servaes, A. Miranda, S. Stroobants, and S. Staelens. 2016. Performance Characterization of an Actively Cooled Repetitive Transcranial Magnetic Stimulation Coil for the Rat. Neuromodulation: journal of the International Neuromodulation Society 19(5):459-468.
Paszek M J, DuFort C C, Rossier O, Bainer R, Mouw J K, Godula K, et al. The cancer glycocalyx mechanically primes integrin-mediated growth and survival. Nature. (2014) 511:319-25. doi: 10.1038/nature13535
Patel S A, Nilsson M B, Le X, Cascone T, Jain R K, Heymach J V. Molecular mechanisms and future implications of VEGF/VEGFR in cancer therapy. Clin Cancer Res. (2023) 29:30-9. doi: 10.1158/1078-0432.CCR-22-1366
Peng W, Polajžer T, Yao C, D. Miklavčič. Dynamics of cell death due to electroporation using different pulse parameters as revealed by different viability assays. Ann Biomed Eng. (2024) 52:22-35. doi: 10.1007/s10439-023-03309-8
Pinho, S. S., and C. A. Reis. 2015. Glycosylation in cancer: mechanisms and clinical implications. Nature reviews. Cancer 15(9):540-555.
Polajzer T, Jarm T, Miklavcic D. Analysis of damage-associated molecular pattern molecules due to electroporation of cells in vitro. Radiol Oncol. (2020) 54:317-28. doi: 10.2478/raon-2020-0047
Polk C, Postow E. Handbook of Biological Effects of Electromagnetic Fields,
Third Edition—2 Volume Set. Taylor & Francis, Boca Raton, FL: (1995).
Preet Kaur A, Alice A, Crittenden M R, Gough M J. The role of dendritic cells in radiation-induced immune responses. Int Rev Cell Mol Biol. (2023) 378:61-104. doi: 10.1016/bs.ircmb.2023.02.002
Qian C, Liu C, Liu W, Zhou R, Zhao L. Targeting vascular normalization: a promising strategy to improve immune-vascular crosstalk in cancer immunotherapy. Front Immunol. (2023) 14:1291530. doi: 10.3389/fimmu.2023.1291530
Radeva M, Berg H. Differences in lethality between cancer cells and human lymphocytes caused by LF-electromagnetic fields. Bioelectromagnetics. (2004) 25:503-7. doi: 10.1002/bem.20023
Reale, M., M. A. Kamal, A. Patruno, E. Costantini, C. D'Angelo, M. Pesce, and N. H. Greig. 2014. Neuronal cellular responses to extremely low frequency electromagnetic field exposure: implications regarding oxidative stress and neurodegeneration. PloS one 9(8):e104973.
Rodrigues, E., and M. S. Macauley. 2018. Hypersialylation in Cancer: Modulation of Inflammation and Therapeutic Opportunities. Cancers (Basel) 10(6).
Rominiyi, O., A. Vanderlinden, S. J. Clenton, C. Bridgewater, Y. Al-Tamimi, and S. J. Collis. 2021. Tumour treating fields therapy for glioblastoma: current advances and future directions. British journal of cancer 124(4):697-709.
Saddawi-Konefka R, O'Farrell A, Faraji F, Clubb L, Allevato M M, Jensen S M, et al. Lymphatic-preserving treatment sequencing with immune checkpoint inhibition unleashes cDC1-dependent antitumor immunity in HNSCC. Nat Commun. (2022) 13:4298. doi: 10.1038/s41467-022-31941-w
Sarrazin, S., W. C. Lamanna, and J. D. Esko. 2011. Heparan sulfate proteoglycans. Cold Spring Harb Perspect Biol 3(7).
Sasaki, N., M. Toyoda, and T. Ishiwata. 2021. Gangliosides as Signaling Regulators in Cancer. International journal of molecular sciences 22(10).
Scheer M, Bork K, Simon F, Nagasundaram M, Horstkorte R, Gnanapragassam V S: Glycation Leads to Increased Polysialylation and Promotes the Metastatic Potential of Neuroblastoma Cells. Cells 2020, 9(4).
Seidenfaden, R., A. Krauter, F. Schertzinger, R. Gerardy-Schahn, and H. Hildebrandt. 2003. Polysialic acid directs tumor cell growth by controlling heterophilic neural cell adhesion molecule interactions. Mol Cell Biol 23(16):5908-5918.
Sharma A, Jasrotia S, Kumar A. Effects of chemotherapy on the immune system: implications for cancer treatment and patient outcomes. Naunyn-Schmiedeberg's Arch Pharmacol. (2024) 397:2551-66. doi: 10.1007/s00210-023-02781-2
Sharma S, Wu S Y, Jimenez H, Xing F, Zhu D, Liu Y, et al. Ca(2+) and CACNAlH mediate targeted suppression of breast cancer brain metastasis by AM RF EMF. EBioMedicine. (2019) 44:194-208. doi: 10.1016/j.ebiom.2019.05.038
Siegel R L, Miller K D, Fuchs H E, Jemal A: Cancer statistics, 2022. CA: a cancer journal for clinicians 2022, 72(1):7-33.
Son R S, Smith K C, Gowrishankar T R, Vernier P T, Weaver J C. Basic features of a cell electroporation model: illustrative behavior for two very different pulses. J Membrane Biol. (2014) 247:1209-28. doi: 10.1007/s00232-014-9699-z
Sorensen T H, Cruys-Bagger N, Borch K, Westh P. Free energy diagram for the heterogeneous enzymatic hydrolysis of glycosidic bonds in cellulose. J Biol Chem. (2015) 290:22203-11. doi: 10.1074/jbc.M115.659656
Sorokin, M., I. Kholodenko, D. Kalinovsky, T. Shamanskaya, I. Doronin, D. Konovalov, A. Mironov, D. Kuzmin, D. Nikitin, S. Deyev, A. Buzdin, and R. Kholodenko. 2020. RNA Sequencing-Based Identification of Ganglioside G D2-Positive Cancer Phenotype. Biomedicines 8(6).
Suzuki M, Suzuki M, Nakayama J, Suzuki A, Angata K, Chen S, et al. Polysialic acid facilitates tumor invasion by glioma cells. Glycobiology. (2005) 15:887-94. doi: 10.1093/glycob/cwi071
Szklener K, Bilski M, Nieoczym K, Mańdziuk D, Mańdziuk S. Enhancing glioblastoma treatment through the integration of tumor-treating fields. Front Oncol. (2023) 13:1274587. doi: 10.3389/fonc.2023.1274587
Tang D, Kang R. Tarlatamab: the promising immunotherapy on its way from the lab to the clinic. Trans Lung Cancer Res. (2023) 12:1355-7. doi: 10.21037/tlcr
Tatarov, I., A. Panda, D. Petkov, K. Kolappaswamy, K. Thompson, A. Kavirayani, M. M. Lipsky, E. Elson, C. C. Davis, S. S. Martin, and L. J. DeTolla. 2011. Effect of magnetic fields on tumor growth and viability. Comparative medicine 61(4):339-345.
Tofani S. Magnetic fields and apoptosis: a possible mechanism. Electromagnetic Biol Med. (2022) 41:293-303. doi: 10.1080/15368378.2022.2073547
Tofani, S., D. Barone, M. Cintorino, M. M. de Santi, A. Ferrara, R. Orlassino, P. Ossola, F. Peroglio, K. Rolfo, and F. Ronchetto. 2001. Static and ELF magnetic fields induce tumor growth inhibition and apoptosis. Bioelectromagnetics 22(6):419-428.
Tsioptsias C. Thermodynamic and vibrational aspects of peptide bond hydrolysis and their potential relationship to the harmfulness of infrared radiation. Molecules (Basel Switzerland). (2023) 28:1-12. doi: 10.3390/molecules28237902
Vadalà M, Morales-Medina J C, Vallelunga A, Palmieri B, Laurino C, Iannitti T. Mechanisms and therapeutic effectiveness of pulsed electromagnetic field therapy in oncology. Cancer Med. (2016) 5:3128-39. doi: 10.1002/cam4.861
Valentiner U, Mühlenhoff M, Lehmann U, Hildebrandt H, Schumacher U: Expression of the neural cell adhesion molecule and polysialic acid in human neuroblastoma cell lines. Int J Oncol 2011, 39(2):417-424.
Vasishta V G. Sequentially programmed magnetic field therapy in the management of recurrent anaplastic astrocytoma: A case report and literature review. Case Rep Oncol. (2010) 3:189-94. doi: 10.1159/000316358
Vasseur J A, Goetz J A, Alley W R, Jr., Novotny M V: Smoking and lung cancer-induced changes in N-glycosylation of blood serum proteins. Glycobiology 2012, 22(12):1684-1708.
Vlodavsky I, Kayal Y, Hilwi M, Soboh S, Sanderson R D, Ilan N. Heparanase-A single protein with multiple enzymatic and nonenzymatic functions. Proteoglycan Res. (2023) 1:e6. doi: 10.1002/pgr2.6
Wade, A., A. E. Robinson, J. R. Engler, C. Petritsch, C. D. James, and J. J. Phillips. 2013. Proteoglycans and their roles in brain cancer. The FEBS journal 280(10):2399-2417.
Wang H, Zhang X. Magnetic fields and reactive oxygen species. Int J Mol Sci. (2017) 18:1-20. doi: 10.3390/ijms18102175
Wang Y, Jiang T, Xie L, Wang H, Zhao J, Xu L, et al. Effect of pulsed field ablation on solid tumor cells and microenvironment. Front Oncol. (2022) 12:899722. doi: 10.3389/fonc.2022.899722
Wang Y, Shao Q, Van de Moortele P F, Racila E, Liu J, Bischof J, et al. Mapping electrical properties heterogeneity of tumor using boundary informed electrical properties tomography (BIEPT) at 7T. Magnetic Resonance Med. (2019) 81:393-409. doi: 10.1002/mrm.27414
Wang, S., Y. Hu, Y. Yan, Z. Cheng, and T. Liu. 2018. Sotetsuflavone inhibits proliferation and induces apoptosis of A549 cells through ROS-mediated mitochondrial-dependent pathway. BMC complementary and alternative medicine 18(1):235.
Wielgat, P., K. Niemirowicz-Laskowska, A. Z. Wilczewska, and H. Car. 2021. Sialic Acid-Modified Nanoparticles-New Approaches in the Glioma Management-Perspective Review. International journal of molecular sciences 22(14).
Williams C D, Markov M S, Hardman W E, Cameron I L: Therapeutic electromagnetic field effects on angiogenesis and tumor growth. Anticancer research 2001, 21(6a):3887-3891.
Wylie P G: Multiple cell lines using quantum dots. Methods in molecular biology (Clifton, NJ) 2007, 374:113-123.
Xu A, Wang Q, Lv X, Lin T. Progressive study on the non-thermal effects of magnetic field therapy in oncology. Front Oncol. (2021) 11:638146. doi: 10.3389/fonc.2021.638146
Yang H, Wang L. Heparan sulfate proteoglycans in cancer: Pathogenesis and therapeutic potential. Adv Cancer Res. (2023) 157:251-91. doi: 10.1016/bs.acr.2022.08.001
Yayon, A., M. Klagsbrun, J. D. Esko, P. Leder, and D. M. Ornitz. 1991. Cell surface, heparin-like molecules are required for binding of basic fibroblast growth factor to its high affinity receptor. Cell 64(4):841-848.
Yue, J., W. Jin, H. Yang, J. Faulkner, X. Song, H. Qiu, M. Teng, P. Azadi, F. Zhang, R. J. Linhardt, and L. Wang. 2021. Heparan Sulfate Facilitates Spike Protein-Mediated SARS-CoV-2 Host Cell Invasion and Contributes to Increased Infection of SARS-CoV-2 G614 Mutant and in Lung Cancer. Frontiers in molecular biosciences 8:649575.
Zadeh-Haghighi H, Simon C. Magnetic field effects in biology from the perspective of the radical pair mechanism. J R Society Interface. (2022) 19:20220325. doi: 10.1098/rsif.2022.0325
Zhang N, Li Z, Han X, Zhu Z, Li Z, Zhao Y, et al. Irreversible electroporation: an emerging immunomodulatory therapy on solid tumors. Front Immunol. (2021) 12:811726. doi: 10.3389/fimmu.2021.811726
Zhang X, Zhang H, Zheng C, Li C, Zhang X, Xiong W. Extremely low frequency (ELF) pulsed-gradient magnetic fields inhibit Malignant tumour growth at different biological levels. Cell Biol Int. (2002) 26:599-603. doi: 10.1006/cbir.2002.0883
Zhang, X., H. Nie, J. Whited, D. Wang, Y. Li, and X. L. Sun. 2018. Recent approaches for directly profiling cell surface sialoform. Glycobiology 28(12):910-924.
Zhao Y, Shen M, Wu L, Yang H, Yao Y, Yang Q, et al. Stromal cells in the tumor microenvironment: accomplices of tumor progression?Cell Death Dis. (2023) 14:587. doi: 10.1038/s41419-023-06110-6
Zheng Y, Ma X, Su D, Zhang Y, Yu L, Jiang F, et al. The roles of siglec7 and siglec9 on natural killer cells in virus infection and tumour progression. J Immunol Res. (2020) 2020:6243819. doi: 10.1155/2020/6243819
Zhou Y, Xing X, Zhou J, Jiang H, Cen P, Jin C, et al. Therapeutic potential of tumor treating fields for Malignant brain tumors. Cancer Rep (Hoboken N J). (2023) 6:e1813. doi: 10.1002/cnr2.1813
Zitvogel L, Apetoh L, Ghiringhelli F, Kroemer G. Immunological aspects of cancer chemotherapy. Nat Rev Immunol. (2008) 8:59-73. doi: 10.1038/nri2216

1. A method of treating a neoplastic malignant cell cancer in a subject in need thereof comprising:
a) obtaining or having obtained a sample from the subject, wherein the sample comprises at least one neoplastic malignant cancer cell;
b) determining that the isolated neoplastic malignant cancer cell comprises a malignant glycocalyx B; and
c) applying a pulsed magnetic field (PMF) to the subject, thereby treating the neoplastic malignant cell cancer in the subject.

2. The method of claim 1, further comprising administering a therapeutically effective amount an immunotherapy to the subject.

3. The method of claim 2, wherein the therapeutically effective amount the immunotherapy is administered before, during, and/or after applying the pulsed magnetic field to the subject.

4. The method of claim 1, further comprising administering a therapeutically effective amount of chemotherapy to the subject.

5. The method of claim 4, wherein the therapeutically effective amount the chemotherapy is administered before, during, and/or after applying the pulsed magnetic field to the subject.

6. The method of claim 1, wherein the PMF is administered daily for about 7 days.

7. The method of claim 2, wherein the PMF is administered daily for about 7 days during each round of immunotherapy.

8. The method of claim 1, wherein the PMF is administered at an amplitude of about 5-30 mT.

9. The method of claim 1, wherein the PMF is administered at a 20 mT maximum field (dB/dt˜2 T/sec).

10. The method of claim 1, wherein the PMF is administered at a frequency of about 50 Hz to 385 Hz.

11. The method of claim 1, wherein the PMF is 20 mT maximum field (dB/dt˜2 T/sec) over a 10 msec duty cycle at 50 Hz.

12. The method of claim 1, wherein the PMF is administered at a duty-cycle pulse width of 70 μsec using 15 Hz pulse trains.

13. The method of claim 1, wherein the PMF is administered at a pulse duty-cycle rise time of about 5-15 msec.

14. The method of claim 1, further comprising administering radiation therapy to the subject.

15. The method of claim 14, wherein the radiation therapy is administered before, during, and/or after applying the PMF.

16. The method of claim 1, wherein the isolated neoplastic malignant cancer cell does not comprise a malignant glycocalyx A.

17. A method of inducing apoptosis of one or more neoplastic malignant cells comprising:
a) determining that the one or more neoplastic malignant cells comprises a malignant glycocalyx B; and
b) applying a pulsed magnetic field (PMF) to the one or more neoplastic malignant cells, thereby inducing apoptosis of one or more of the neoplastic malignant cells.

18. The method of claim 17, wherein the one or more neoplastic malignant cells are in culture or in a subject.

19. (canceled)

20. The method of claim 17, further comprising exposing the one or more neoplastic malignant cells to a therapeutically effective amount an immunotherapy or chemotherapy.

21.-34. (canceled)

35. A method of increasing a subject's responsiveness to an immunotherapy, comprising:
a. obtaining or having obtained a sample from the subject, wherein the sample comprises at least one neoplastic malignant cancer cell;
b. determining that the isolated neoplastic malignant cancer cell comprises a malignant glycocalyx B; and
c. applying a pulsed magnetic field (PMF) to the subject, thereby increasing a subject's responsiveness to an immunotherapy.

36.-50. (canceled)
